SEQUENCE LISTING (49 sequences)

SEQ ID NO: 1 - DNA, 549 bp, Synthetic construct
  source 1..549: mol_type = other DNA
atgggcggaggagcccctacccaggacgagttcacccagctgagccagagcatcgctgagttccacacctaccagctgggaaacggacgctgttccagcctgctggcacagagaatccacgctcctcctgagacagtgtggagtgtggtgcgcagattcgaccgccctcagatttacaagcacttcatcaagagctgcaacgtgagcgaggacttcgagatgagagtgggatgtaccagagatgtgaacgtgatcagcggactgcctgccaacaccagcagagagagactggacctgctggacgatgaccgcagagtgaccggcttcagcatcaccggaggtgagcacagactgagaaactacaagagcgtgaccaccgtccaccgcttcgagaaggaagaggaagaggagcgcatctggaccgtggtgctggagagctacgtcgtggacgtgcccgagggcaacagcgaagaggatacccgcctgttcgctgacaccgtgatcagactgaacctccagaagctggccagcatcaccgaggcaatgaac

SEQ ID NO: 2 - protein, 183 aa, synthetic construct
MGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 3 - protein, 177 aa, synthetic construct
TQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 4 - skipped

SEQ ID NO: 5 - DNA, 903 bp, Synthetic construct
  source 1..903: mol_type = other DNA
atgaccagagtgcccctgtacggcttcaccagcatttgtggcagacggcccgaaatggaagccgccgtgtctacaatccccagattcctccagagcagcagcggctccatgctggacggcagattcgatcctcagagcgccgctcacttcttcggcgtgtacgatggacatggcggaagccaggtggccaactactgccgcgaaagaatgcatctggccctggccgaggaaatcgccaaagaaaagcccatgctgtgcgacggcgacacctggctggaaaagtggaagaaggccctgttcaacagcttcctgagagtggacagcgagatcgagagcgtggcccctgaaacagtgggcagcacatctgtggtggccgtggtgtttcccagccacatcttcgtggctaactgcggcgatagcagagccgtgctgtgcagaggaaaaacagccctgcctctgtccgtggaccacaagcctgatagagaggatgaggccgccagaattgaagccgctggcggcaaagtgatccagtggaatggcgctagagtgttcggcgtgctggccatgagtagatccatcggcgatagatacctgaagcctagcatcatccccgatcctgaagtgaccgccgtgaagagagtgaaagaggacgactgcctgatcctggcctctgacggtgtctgggacgtgatgacagatgaagaggcctgcgagatggcccggaagagaatcctgctgtggcacaagaaaaacgccgtggccggggatgcttctctgctggctgacgagagaagaaaagagggcaaagaccccgctgccatgtctgccgccgagtacctgtctaagctggccatccagagaggcagcaaggacaacatcagcgtggtggtcgtggacctgaaa

SEQ ID NO: 6 - protein, 301 aa, Synthetic construct
MTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 7 - protein, 298 aa, Synthetic construct
VPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 8 - skipped

SEQ ID NO: 9 - DNA, 45 bp, Synthetic construct
  source 1..45: mol_type = other DNA
actagtggcggcggaggcagcggaggcggatctggcggaggatct

SEQ ID NO: 10 - protein, 15 aa, Synthetic construct
TSGGGGSGGGSGGGS

SEQ ID NO: 11 - DNA, 45 bp, Synthetic construct
  source 1..45: mol_type = other DNA
acgcgtggcggcggaggcagcggaggcggatctggcggaggatct

SEQ ID NO: 12 - protein, 15 aa, Synthetic construct
TRGGGGSGGGSGGGS

SEQ ID NO: 13 - DNA, 45 bp, Synthetic construct
  source 1..45: mol_type = other DNA
ggcggcggaggaagtggcggcggatctggcggaggatctaccggt

SEQ ID NO: 14 - protein, 15 aa, Synthetic construct
GGGGSGGGSGGGSTG

SEQ ID NO: 15 - protein, 23 aa, Synthetic construct
DQSNSEEAKKEEAKKEEAKKSNS

SEQ ID NO: 16 - protein, 40 aa, Synthetic construct
SGGGSGGGSGGGSGGSGGSGGGSGGSGGSGGGSGGGSGGG

SEQ ID NO: 17 - protein, 12 aa, Synthetic construct
ESKYGPPAPPAP

SEQ ID NO: 18 - protein, 49 aa, Synthetic construct
AVGQDTQEVIVVPHSLPFKVVVISAILALVVLTIISLIILIMLWQKKPR

SEQ ID NO: 19 - DNA, 147 bp, Synthetic construct
  source 1..147: mol_type = other DNA
gccgtcggccaggacacccaagaagtgatcgtcgtccctcacagcctgcctttcaaggtggtggtcatcagcgccattctggccctggtggtgctgaccatcatcagcctgatcatcctgattatgctgtggcagaagaagcccaga

SEQ ID NO: 20 - protein, 244 aa, Synthetic construct
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRGGGGSGGGGSGGGGSQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSS

SEQ ID NO: 21 - DNA, 732 bp, Synthetic construct
  source 1..732: mol_type = other DNA
aacatcatgatgacgcagagccccagcagcctggctgtttctgctggcgagaaagtgaccatgacctgcaagagcagccagagcgtgctgtactccagcaaccagaagaactacctggcctggtatcagcagaagcccggccagtctcctaagctgctgatctactgggccagcaccagagaaagcggcgtgcccgatagattcacaggctctggcagcggcaccgacttcaccctgacaatcagtagcgtgcagcccgaggatctggccgtgtactactgtcaccagtacctgagcagccacacctttggcggcggaacaaagctggaaatcaagagaggcggaggcggatcaggtggcggtggatctggcggtggtggatctcaacttcagcagccaggcgcagaacttgtgcggcctggatctagcgtgaagctgagctgtaaagccagcggctacaccttcaccagatactggatccactgggtcaagcagcggcctatccagggactcgagtggatcggcaatatcgaccccagcgacagcgagacacactacaatcagaagttcaaggacaaggccacactgaccgtggacaagtctagcggcacagcctacatgcagctgtccagcctgacaagcgaggacagcgccgtgtattattgcgccaccgaggacctgtactacgccatggaatattggggccagggcaccagcgtgaccgttagctct

SEQ ID NO: 22 - protein, 26 aa, Synthetic construct
IASFFFIIGLIIGLFLVLRVGIHLCI

SEQ ID NO: 23 - DNA, 78 bp, Synthetic construct
  source 1..78: mol_type = other DNA
attgcctcttttttctttatcatagggttaatcattggactattcttggttctccgagttggtatccatctttgcatt

SEQ ID NO: 24 - protein, 184 aa, synthetic construct
CPSQCSCSGTEVHCQRKSLASVPAGIPTTTRVLYLHVNEITKFEPGVFDRLVNLQQLYLGGNQLSALPDGVFDRLTQLTRLDLYNNQLTVLPAGVFDRLVNLQTLDLHNNQLKSIPRGAFDNLKSLTHIWLFGNPWDCACSDILYLSGWLGQHAGKEQGQAVCSGTNTPVRAVTEASTSPSKCP

SEQ ID NO: 25 - DNA, 552 bp, synthetic construct
  source 1..552: mol_type = other DNA
tgccccagccagtgcagctgctccggcacagaagtgcattgccagagaaagtccctggcctctgtgcctgccggcattcctaccacaaccagagtgctgtacctgcacgtgaacgagatcaccaagttcgagcccggcgtgttcgacagactggtcaatctccagcagctgtacctcggcggcaatcagctttctgctctgcccgatggggtgttcgataggctgacccagctgaccagactggacctgtataacaatcagctgaccgtgctgccagccggcgttttcgatcggctcgtgaatctccagactctggacctgcacaacaaccagttgaagtctatccccagaggggccttcgacaacctgaagtctctgacccacatctggctgttcggcaacccctgggattgcgcctgtagcgacatcctgtatctgtctggctggctgggacagcacgccggcaaagaacaaggacaggctgtgtgcagcggcaccaatactccagtcagagccgtgaccgaggccagcacaagcccttctaaatgccct

SEQ ID NO: 26 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 27 - DNA, 1533 bp, synthetic construct
  source 1..1533: mol_type = other DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaag

SEQ ID NO: 28 - protein, 511 aa, synthetic construct
MKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 29 - DNA, 1533 bp, Synthetic construct
  source 1..1533: mol_type = other DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgccccagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaagcggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaag

SEQ ID NO: 30 - protein, 448 aa, Synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSS

SEQ ID NO: 31 - DNA, 1533 bp, Synthetic construct; identical to SEQ ID NO: 27
  source 1..1533: mol_type = other DNA

SEQ ID NO: 32 - protein, 448 aa, Synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSS

SEQ ID NO: 33 - DNA, 1392 bp, Synthetic construct
  source 1..1392: mol_type = other DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgccccagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaagcggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctct

SEQ ID NO: 34 - protein, 16 aa, Synthetic construct
MKCLLYLAFLFIGVNC

SEQ ID NO: 35 - DNA, 48 bp, Synthetic construct
  source 1..48: mol_type = other DNA
atgaagtgccttttgtacttagcctttttattcattggggtgaattgc

SEQ ID NO: 36 - protein, 21 aa, Synthetic construct
KLKHTKKRQIYTDIEMNRLGK

SEQ ID NO: 37 - DNA, 63 bp, Synthetic construct
  source 1..63: mol_type = other DNA
aaattaaagcacaccaagaaaagacagatttatacagacatagagatgaaccgacttggaaag

SEQ ID NO: 38 - protein, 21 aa, synthetic construct
METDTLLLWVLLLWVPGSTGD

SEQ ID NO: 39 - DNA, 63 bp, Synthetic construct
  source 1..63: mol_type = other DNA
atggaaacggacaccctgctgctgtgggtgctgttgttgtgggtgccaggatctacaggcgac

SEQ ID NO: 40 - protein, 22 aa, Synthetic construct
DYKDHDGDYKDHDIDYKDDDDK

SEQ ID NO: 41 - DNA, 66 bp, Synthetic construct
  source 1..66: mol_type = other DNA
gattacaaggaccacgatggcgactataaggatcacgacatcgactacaaggacgatgacgacaag

SEQ ID NO: 42 - protein, 75 aa, synthetic construct
  VARIANT 1..75: note = GGGGS repeats may vary between 1-15. Up to 14 GGGGS repeats may be absent
GGGGSGGGGSGGGGSGGGGSGGGGSGGGGSGGGGSGGGGSGGGGSGGGGSGGGGSGGGGSGGGGSGGGGSGGGGS

SEQ ID NO: 43 - RNA, 20 nt, synthetic construct
  source 1..20: mol_type = other RNA
gaagcgtgatgacaaagagg

SEQ ID NO: 44 - DNA, 59 bp, synthetic construct
  source 1..59: mol_type = other DNA
  misc_difference 31..35: note = N can be A or G or C or T
acactctttccctacacgctcttccgatctnnnnngagaagcatgacggacaagtacag

SEQ ID NO: 45 - DNA, 61 bp, synthetic construct
  source 1..61: mol_type = other DNA
  misc_difference 35..39: note = N can be A or G or C or T
gtgactggagttcagacgtgtgctcttccgatctnnnnntcccaaagtaccagtttgccac

SEQ ID NO: 46 - DNA, 55 bp, synthetic construct
  source 1..55: mol_type = other DNA
aatgatacggcgaccgagatctacactctttccctacacgacgctcttccgatct

SEQ ID NO: 47 - DNA, 49 bp, synthetic construct
  source 1..49: mol_type = other DNA; note = the sequence contains Index
caagcagaagacggcatacgagatgtgactggagttcagacgtgtgctc

SEQ ID NO: 48 - DNA, 64 bp, synthetic construct
  source 1..64: mol_type = other DNA
gctgcacctgtcagtggccgacctcctctttgtcatcacgcttcccttctgggcagttgatgcc

SEQ ID NO: 49 - DNA, 64 bp, synthetic construct
  source 1..64: mol_type = other DNA
cgacgtggacagtcaccggctggaggagaaacagtagtgcgaagggaagacccgtcaactacgg

(19) United States
(12) Patent Application Publication
(10) Pub. No.: US 2025/0312479 A1
(43) Pub. Date: Oct. 9, 2025

(54) RECEPTOR ENGAGEMENT-MEDIATED ENHANCEMENT OF BIOLOGICS DELIVERY

(71) Applicant: Northwestern University, Evanston, IL (US)

(72) Inventors: John D. Rosanelli, Jr., Chicago, IL (US); Hailey Ilyse Edelstein, Evanston, IL (US); Joshua N. Leonard, Evanston, IL (US); Devin Stranford, Evanston, IL (US)

(73) Assignee: Northwestern University, Evanston, IL (US)

(21) Appl. No.: 18/865,155

(22) PCT Filed: May 13, 2023
(86) PCT No.: PCT/US2023/022166

§ 371 Date: Nov. 12, 2024

Related U.S. Application Data
(60) Provisional application No. 63/341,914, filed on May 13, 2022.

(51) Int. Cl. A61K 47/69; A61K 40/31; A61K 47/10; A61K 47/68; A61K 48/00; C07K 14/47; C07K 14/705; C12N 15/88

(52) U.S. Cl. CPC A61K 47/6901; A61K 40/31; A61K 47/10; A61K 47/68; A61K 47/6907; A61K 48/005; C07K 14/4702; C07K 14/705; C12N 15/88; C07K 2319/03; C07K 2319/70

(57) ABSTRACT

The present disclosure provides methods and compositions for targeting lipid bilayer particles, such as secreted extracellular vesicles, and cargo entities included therein to recipient cells.

### CROSS-REFERENCE TO RELATED APPLICATION

This application claims priority to U.S. provisional application No. 63/341,914, filed on May 13, 2022, which is incorporated herein by reference in its entirety.

The instant application contains a Sequence Listing which has been submitted in XML format and is hereby incorporated by reference in its entirety. Said XML copy, created on Jul. 13, 2023, is named 121384-0202.xml and is 56,984 bytes.

### STATEMENT OF GOVERNMENT SUPPORT

This invention was made with government support under Grant No. P30 All 17943 awarded by the National Institutes of Health. The United States government has certain rights in the invention.

### TECHNICAL FIELD

The present disclosure relates generally to methods and compositions for loading cargo entities into secreted lipid bilayer particles (e.g., cell-derived membrane particles such as extracellular vesicles).

### BACKGROUND

The burgeoning field of nucleic acid therapeutics has garnered much attention, among other things because of its potential to treat a variety of diseases, disorders, or conditions that may not be readily addressable by other modalities. However, as noted in a recent review, “[w]hile nucleic acid therapeutics can expand the array of treatable diseases, their broader use is limited by multiple delivery challenges” (Gupta et al., “Nucleic acid delivery for therapeutic applications” in Adv. Drug Delivery Reviews 178:113834, 2021).

The following discussion is merely provided to aid the reader in understanding the disclosure and is not admitted to describe or constitute prior art thereto.

Secreted extracellular vesicles (EVs), such as exosomes and microvesicles, are nanometer-scale lipid vesicles that are produced by many cell types and transfer proteins, nucleic acids, and other molecules between cells in the human body, as well as those of other animals. A number of viruses are known to leverage the EVs to deliver their genomes to other cells (e.g., enveloped viruses). EVs have a wide variety of potential therapeutic uses and are an attractive platform for delivering a wide variety of therapeutics. For example, targeted exosomes have already been shown to be effective for delivery of RNA to neural cells and tumor cells in mice. Other cell-derived membrane particles can also be used for similar purposes.

Controlling the composition of EVs, both in the lumen and/or membrane, is challenging. Various passive approaches exist for both but there is need for superior engineering of EV compositions to achieve their therapeutic potential. The disclosed technology aims to address these limitations of the current technologies.

### SUMMARY OF THE PRESENT TECHNOLOGY

The present disclosure provides a remarkable new technology for achieving delivery of a cargo (e.g., a nucleic acid cargo, polypeptide cargo, a nucleocapsid cargo, and combinations thereof) to a recipient cell or cell population using engineered lipid bilayer particles.

Among other things, the present disclosure provides an insight that a combination of certain affinity agent polypeptides and fusogen agent polypeptides on surfaces of lipid bilayer particles can confer surprising improved delivery attributes to such particles, for example achieving more efficient delivery and/or delivery to particular cell types.

The present disclosure identifies the source of one or more problems associated with many conventional strategies for payload delivery (e.g., nucleic acid payload delivery) to recipient cell(s) of interest, including specifically with technologies intended to achieve in vivo delivery. Among other things, the present disclosure identifies the source of a particular problem associated with conventional strategies that utilize a viral fusogen (e.g., VSV-G), or a variant thereof, to achieve payload delivery; the present disclosure demonstrates that combining a fusogen entity polypeptide (e.g., a viral fusogen entity polypeptide) with a targeting chimeric polypeptide, as described herein, solves such identified problem(s) and/or otherwise achieves particularly beneficial results (e.g., particularly precise and/or efficient payload delivery).

The present disclosure specifically appreciates challenges associated with effective payload (e.g., nucleic acid payload) delivery to certain immune cells, e.g., T cells. The present disclosure documents particular effectiveness of provided technologies in delivering payload (e.g., nucleic acid payload) to T cells, e.g., to activated T cells.

Among other things, the present disclosure provides engineered lipid bilayer particles, and preparations thereof, whose surfaces include both a fusogen entity polypeptide and a targeting chimeric polypeptide as described herein. In some embodiments, such provided particles and/or preparations are characterized by particular payload delivery attributes. In some embodiments, such provided particles and/or preparations achieve payload delivery (e.g., specific payload delivery and/or enhanced payload delivery) to particular cell(s) or cell population(s) of interest; in some embodiments such delivery is in vivo. The present disclosure provides, among other things, particular combinations of a fusogen entity polypeptide and a targeting chimeric polypeptide, as described herein, can drive specific functions.

In some embodiments, provided technology is useful for delivery of viral vectors (e.g., lentivirus cores, adeno-associated virus particles), and/or virus-like particles within lipid bilayer particles. Alternatively or additionally, in some embodiments, provided technologies are useful for delivery of non-viral vectors (e.g., nucleic acid payloads that are not packaged within a protein core or capsid structure).

Among other things, the present disclosure identifies challenges with in vivo gene delivery to cells, including specifically to certain immune system cells (e.g., T cells), in particular in vivo delivery of a cargo (e.g., payload) for specifically and efficiently targeting particular recipient cells of interest (e.g., T cells). In vitro delivery of a cargo (e.g., payload) to target cells, and in particular to T cells, in a specific and efficient fashion is partially met by some methods, but there remains unmet need for specific in vivo delivery and non-toxic in vivo and in vitro delivery, as well as an unmet need for more efficient in vitro delivery to cells.

Among other things, in some embodiments, the present disclosure provides technologies (e.g., systems, engineered lipid bilayer parties, production cells, method of manufacturing and delivery) that mediate fusion of an engineered lipid bilayer particle to a recipient cell (e.g., to deliver a cargo).

Certain particularly useful applications of provided technologies include, for example, CAR T cell therapy, for example in the manufacture of CAR T cells for oncology treatment, immune system disorders, and other applications.

Among other things, in some embodiments, the present disclosure provides technologies that enhance delivery of a particular cargo (e.g., payload) and/or delivery to a particular recipient cell or cell populations, including specifically to certain immune cells or cell populations and in in particular to T cells or T cell populations.

In some embodiments, the present disclosure achieves specificity and/or efficiency of payload delivery through combined activity of a fusogen entity polypeptide and a targeting chimeric polypeptide. In some embodiments, provided technologies achieve delivery that shows greater specificity and/or efficiency when compared with a particular reference; in some embodiments, such reference may be a sufficiently comparable system including one or the other of the fusogen entity polypeptide and the targeting chimeric polypeptide, but not both. In many embodiments, an appropriate reference may be a sufficiently comparable system including the fusogen entity polypeptide but not the targeting chimeric polypeptide. Alternatively or additionally, in some embodiments, an appropriate reference may be a sufficiently comparable system that includes a particular viral fusogen entity polypeptide (e.g., VSV-G or a variant thereof) and, for example, lacks a targeting chimeric polypeptide as described herein. In some embodiments, an appropriate reference does not utilize the same affinity agent polypeptide, even if it includes at least one surface agent with some degree of affinity for surfaces of recipient cells or populations thereof.

The present disclosure provides targeting chimeric polypeptides, fusogen entity polypeptides, as well as systems and methods for using the same, for targeting cargo entities into lipid bilayer particles, such as cell-derived membrane particles, including but not limited to, extracellular vesicles. The present disclosure also provides methods of manufacturing engineered production cells, methods of manufacturing preparations of lipid bilayer particles, methods of delivering a cargo entity to a recipient cell, as well as recipient cells containing a cargo entity or cargo entities, which recipient cells may further include a targeting chimeric polypeptide and a fusogen entity polypeptide (e.g., received by fusion of recipient cell membrane with a lipid bilayer particle as described herein), and which recipient cells furthermore have a nucleus.

In one aspect, the present disclosure provides targeting chimeric polypeptides comprising: (a) a targeting domain that binds to a target ligand (e.g., a target ligand present on surfaces of recipient cells of interest; specifically including human cells and/or immune cells such as T cells, furthermore particularly including CD2 and/or CD5, e.g., human CD2 and/or human CD5), wherein the targeting domain comprises an antibody agent such as a Fab, a Fab′, a F(ab′)₂, a Fd, a scFv, a single-chain antibody, a disulfide-linked Fvs (sdFv), a de novo-designed binding molecule, an affinibody, a DARPIN, a nanobody, a variable lymphocyte receptor (VLR), a camelid antibody, etc; and optionally (b) a transmembrane domain. In some embodiments, the targeting domain is a scFv.

In some embodiments, a transmembrane domain comprises AVGQDTQEVIVVPHSLPFKVVVISAILALVVLTIISLIILIMLWQKKPR (SEQ ID NO: 18), a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to SEQ ID NO: 18, or a functional fragment thereof.

In some embodiments, a targeting domain comprises an amino acid sequence of

(SEQ ID NO: 20)
NIMMTQSPSSLAVSAGEKVTMTCKSSQSVLYSSNQKNYLAWYQQKPGQSP
KLLIYWASTRESGVPDRFTGSGSGTDFTLTISSVQPEDLAVYYCHQYLSS
HTFGGGTKLEIKRGGGGSGGGGSGGGGSQLQQPGAELVRPGSSVKLSCKA
SGYTFTRYWIHWVKQRPIQGLEWIGNIDPSDSETHYNQKFKDKATLTVDK
SSGTAYMQLSSLTSEDSAVYYCATEDLYYAMEYWGQGTSVTVSS.

In some embodiments, a targeting domain comprises an amino acid sequence of

(SEQ ID NO: 24)
CPSQCSCSGTEVHCQRKSLASVPAGIPTTTR VLYLHVNEITKFEPGVFD
RLVNLQQLYLGGNQLSALPDGVFDRLTQLTRLDLYNNQLTVLPAGVFDRL
VNLQTLDLHNNQLKSIPRGAFDNLKSLTHIWLFGNPWDCACSDILYLSGW
LGQHAGKEQGQAVCSGTNTPVRAVTEASTSPSKCP.

In some embodiments, a targeting chimeric polypeptide may further comprise a first cargo entity connected to the transmembrane domain via a linker. In some embodiments, the linker comprises:

(1)
an amino acid sequence selected from SEQ ID NO: 10
(TSGGGGSGGGSGGGS),
SEQ ID NO: 12
(TRGGGGSGGGSGGGS),
SEQ ID NO: 14
(GGGGSGGGSGGGSTG),
SEQ ID NO: 15
(DQSNSEEAKKEEAKKEEAKKSNS),
SEQ ID NO: 16
(SGGGSGGGSGGGSGGSGGSGGGSGGSGGSGGGSGGGSGGG),
and
SEQ ID NO: 17
(ESKYGPPAPPAP);
or

(2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 10, 12, 14, 15, 16, or 17.

In another aspect, the present disclosure provides lipid bilayer particles (e.g., cell-derived membrane particles (CDMPs)) comprising a targeting chimeric polypeptide as disclosed herein and/or a fusion entity polypeptide as disclosed herein.

In some embodiments, lipid bilayer particles are CDMPs. In some embodiments, CDMPs are selected from extracellular vesicles, virus particles, virus-like particles (VLPs), apoptotic bodies, platelet-like particles, and combinations thereof. In some embodiments, extracellular vesicles are exosomes, microvesicles and/or combinations thereof.

In some embodiments, a lipid bilayer particle (e.g., a CDMP) comprises a fusogen entity polypeptide. In some embodiments, a fusogen entity polypeptide is a viral polypeptide (e.g., a glycoprotein). In some embodiments, a viral glycoprotein is selected from a lentiviral glycoprotein or a glycoprotein selected from vesicular stomatitis glycoprotein (VSV-G), measles virus glycoprotein H, measles virus glycoprotein F, rabies virus glycoprotein (RVG), gibbon ape leukemia virus glycoprotein (GaLV), amphotropic murine leukemia virus glycoprotein (MLV-A), feline endogenous virus (RD 114) glycoprotein, fowl plague virus (FPV) glycoprotein, Ebola virus (EboV) glycoprotein, vesicular stomatitis virus (VSV) glycoprotein, and lymphocytic choriomeningitis virus (LCMV) glycoprotein. In another aspect, the present disclosure provides lipid bilayer particles that comprise a glycoprotein selected from vesicular stomatitis glycoprotein (VSV-G), measles virus glycoprotein H, measles virus glycoprotein F, rabies virus glycoprotein (RVG), gibbon ape leukemia virus glycoprotein (GaLV), amphotropic murine leukemia virus glycoprotein (MLV-A), feline endogenous virus (RD 114) glycoprotein, fowl plague virus (FPV) glycoprotein, Ebola virus (EboV) glycoprotein, vesicular stomatitis virus (VSV) glycoprotein, lymphocytic choriomeningitis virus (LCMV) glycoprotein, and any combination thereof. The expression of such a glycoprotein or combination of glycoproteins (e.g., measles virus glycoprotein H and measles virus glycoprotein F) can be in an embodiment that is independent of (i.e., does not include) a targeting chimeric polypeptide disclosed herein, as these glycoproteins independently provide novel utility with respect to bind and fusion of lipid bilayer particles to recipient cells.

In some embodiments, a fusogen entity polypeptide is a non-viral polypeptide as described herein.

In some embodiments, a lipid bilayer particle comprises a cargo entity as described herein.

In some embodiments, disclosed lipid bilayer particle (e.g., CDMP) may further comprise a chimeric loading polypeptide comprising a cargo-loading domain comprising an abscisic acid-insensitive 1 (ABI1) sequence, and optionally a cargo entity. In some embodiments, a chimeric loading polypeptide comprises a cargo-loading domain comprising an abscisic acid-insensitive 1 (ABI1) sequence and a cargo entity. In some embodiments, the chimeric loading polypeptide further comprises a linker that connects the cargo entity and the cargo-loading domain. In some embodiments, the linker of the chimeric loading polypeptide comprises an amino acid sequence selected from SEQ ID NO: 10 (TSGGGGSGGGSGGGS), SEQ ID NO: 12 (TRGGGGSGGGSGGGS), SEQ ID NO: 14 (GGGGSGGGSGGGSTG), SEQ ID NO: 15 (DQSNSEEAKKEEAKKEEAKKSNS), SEQ ID NO: 16 (SGGGSGGGSGGGSGGSGGSGGGSGGSGGSGGGSGGGSGGG), and SEQ ID NO: 17 (ESKYGPPAPPAP); or an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 10, 12, 14, 15, 16, or 17.

In some embodiments, the cargo-loading domain of the chimeric loading polypeptide is a truncated variant of a wild-type protein that comprises an extracellular vesicle targeting domain. In some embodiments, the cargo-loading domain of the chimeric loading polypeptide comprises residues 126-423 of wild type ABI1. In some embodiments, the cargo-loading domain of the chimeric loading polypeptide comprises:

(SEQ ID NO: 6)
MTRVPLYGFTSICGRRPEMEAAVSTIPRFLQSSSGSMLDGRFDPQSAAHF
FGVYDGHGGSQVANYCRERMHLALAEEIAKEKPMLCDGDTWLEKWKKALF
NSFLRVDSEIESVAPETVGSTSVVAVVFPSHIFVANCGDSRAVLCRGKTA
LPLSVDHKPDREDEAARIEAAGGKVIQWNGARVFGVLAMSRSIGDRYLKP
SIIPDPEVTAVKRVKEDDCLILASDGVWDVMTDEEACEMARKRILLWHKK
NAVAGDASLLADERRKEGKDPAAMSAAEYLSKLAIQRGSKDNISVVVVDL
K,
(SEQ ID NO: 7)
VPLYGFTSICGRRPEMEAAVSTIPRFLQSSSGSMLDGRFDPQSAAHFFGV
YDGHGGSQVANYCRERMHLALAEEIAKEKPMLCDGDTWLEKWKKALFNSF
LRVDSEIESVAPETVGSTSVVAVVFPSHIFVANCGDSRAVLCRGKTALPL
SVDHKPDREDEAARIEAAGGKVIQWNGARVFGVLAMSRSIGDRYLKPSII
PDPEVTAVKRVKEDDCLILASDGVWDVMTDEEACEMARKRILLWHKKNAV
AGDASLLADERRKEGKDPAAMSAAEYLSKLAIQRGSKDNISVVVVDLK,

a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 6 or 7, or
a functional fragment of SEQ ID NO: 6, SEQ ID NO: 7, or a variant amino acid sequence thereof.

In some embodiments, the cargo entity of the chimeric loading polypeptide is a cytosolic cargo molecule. In some embodiments, the cargo entity of the chimeric loading polypeptide is a membrane-bound cargo entity.

In some embodiments, the first cargo entity is or comprises an ABA-binding sequence. In some embodiments, a first cargo entity is or comprises an ABA-binding sequence comprising a pyrabactin resistance 1-like (PYL1) sequence. In some embodiments, a PYL1 sequence comprises residues 33-209 of wild type PYL1.

In some embodiments a PYL1 sequence comprises

(SEQ ID NO: 2)
MGGGAPTQDEFTQLSQSIAEFHTYQLGNGRCSSLLAQRIHAPPETVWSVV
RRFDRPQIYKHFIKSCNVSEDFEMRVGCTRDVNVISGLPANTSRERLDLL
DDDRRVTGFSITGGEHRLRNYKSVTTVHRFEKEEEEERIWTVVLESYVVD
VPEGNSEEDTRLFADTVIRLNLQKLASITEAMN,
(SEQ ID NO: 3)
TQDEFTQLSQSIAEFHTYQLGNGRCSSLLAQRIHAPPETVWSVVRRFDRP
QIYKHFIKSCNVSEDFEMRVGCTRDVNVISGLPANTSRERLDLLDDDRRV
TGFSITGGEHRLRNYKSVTTVHRFEKEEEEERIWTVVLESYVVDVPEGNS
EEDTRLFADTVIRLNLQKLASITEAMN,

or
a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 2 or 3, or
a functional fragment of SEQ ID NO: 2, SEQ ID NO: 3, or a variant amino acid sequence thereof.

In some embodiments, a lipid bilayer particle disclosed herein may further comprise abscisic acid (ABA).

In some embodiments, a lipid bilayer particle encompasses or contains within it a viral nucleocapsid, a synthetic nucleic acid, a transcription factor, a recombinase, a base editor, prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex. In some embodiments, a cargo entity is selected from the group consisting of a viral nucleocapsid, a synthetic nucleic acid, a transcription factor, a recombinase, a base editor, prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, and a ribonucleoprotein complex.

In another aspect, the present disclosure provides nucleic acids encoding chimeric targeting polypeptides disclosed herein and/or fusogen entity polypeptides.

In another aspect, the present disclosure provides production cells comprising a targeting chimeric polypeptide disclosed herein and/or a fusogen entity polypeptide disclosed herein, a lipid bilayer particle disclosed herein, or a nucleic acid disclosed herein. In some embodiments, a production cell is a mammalian cell. In some embodiments, a mammalian cell is optionally selected from HEK293, HEK293FT, a mesenchymal stem cell, a megakaryocyte, an induced pluripotent stem cell (iPSC), a T cell, an erythrocyte, an erythropoetic precursor, and an iPSC-derived version of any of the preceding cells. In another aspect, the present disclosure provides methods of producing a lipid bilayer particle, comprising culturing a production cell comprising a targeting chimeric polypeptide and/or a fusogen entity polypeptide disclosed herein, a lipid bilayer particle (e.g., a CDMP) disclosed herein, or a nucleic acid disclosed herein, and harvesting lipid bilayer particles (e.g., CDMPs) produced by the cell.

In another aspect, the present disclosure provides methods of targeted delivery of a cargo entity to a recipient cell (e.g., an immune cell such as a lymphocyte), comprising administering to an individual a lipid bilayer particle disclosed herein, wherein the lipid bilayer particle comprises a cargo entity.

In some embodiments, a cargo entity comprises a viral nucleocapsid, a synthetic nucleic acid, a transcription factor, a recombinase, a base editor, a prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex.

In some embodiments, the cargo entity comprises a nucleic acid sequence encoding a chimeric antigen receptor.

In another aspect, the present disclosure provides methods of targeting delivery of a cargo entity to a recipient cell (e.g., an immune cell, such as lymphocyte), comprising obtaining a population of recipient cells (e.g., lymphocytes) from an individual, and contacting the population of recipient cells (e.g., lymphocytes) ex vivo with the lipid bilayer particle disclosed herein, wherein the lipid bilayer particle comprises a cargo entity.

In some embodiments, the population of lymphocytes were obtained via apheresis.

In some embodiments, the ex vivo methods may further comprise administering a population of recipient cells (e.g., lymphocytes) back into the individual after the recipient cells (e.g., lymphocytes) have been contacted with the lipid bilayer particle (e.g., such that the lipid bilayer particles have fused with the recipient cells).

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWING

The Drawing includes the following Figures:

FIG. 1 shows overview of the GEMINI strategy for genetically engineering multifunctional EVs. EV cargo proteins and nucleic acids are expressed in producer cells to facilitate incorporation into multiple vesicle populations: microvesicles, which bud from the cell surface, or exosomes, which are produced by endosomal invaginations into multivesicular bodies. Surface-displayed targeting and fusion proteins aid in binding to and uptake by recipient cells and subsequent cargo release via cell surface fusion or endosomal escape. In the proof-of-principle application explored in this study, the objective is to deliver a Cas9-sgRNA complex to T cells in order to knock out a gene, as described in subsequent sections.

FIGS. 2A-2H show that display of scFvs on EVs mediates specific, targeted binding and uptake to T cells. FIG. 2A. Strategy for targeting EVs to T cells (left) and illustration of EV binding experiments (right). FIG. 2B. Targeted EVs binding to Jurkats (2 h incubation). To evaluate potential differences in dTomato loading, average EV fluorescence was analyzed separately (FIGS. 11A-11H). FIG. 2C. Representative histograms depicting distributions of helical linker EV-mediated fluorescence in recipient cells analyzed in FIG. 2B. FIG. 2D. Distinguishing binding and internalization for EVs targeted to Jurkats. Trypsinization was used to remove bound, non-internalized EVs following a 6 h incubation. FIG. 2E. Specificity of EV targeting to CD2. Pre-incubation with anti-CD2 antibodies ablated EV targeting to Jurkats. FIG. 2F. Enhancement of targeting by codon-optimized expression of scFv constructs. Fold increases over the non-targeted control are reported in blue. FIG. 2G. Binding of targeted EVs to primary human CD4⁺ T cells (2 h incubation). FIG. 2H. Distinguishing binding and internalization for EVs targeted to primary human CD4+ T cells. All experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001). EV dTomato loading evaluations are in FIGS. 11A-11H.

FIGS. 3A-3G show that cargo protein is actively loaded into EVs via tagging with the ABI domain of the abscisic acid dimerization system. FIG. 3A. Illustration of abscisic acid-based dimerization of EV cargo proteins and subsequent loading into vesicles. FIG. 3B. ABA-induced dimerization between PYL and ABI domains. Illustrative microscopy showing anti-CD2 scFv-PYL (membrane bound) and EYFP-ABI (cytosolic) association in the presence of ABA. Full images are in FIGS. 17A-17B. FIG. 3C. ABI-induced cargo loading into EVs. EVs generated under conditions indicated were adsorbed to aldehyde/sulfate latex beads and analyzed by flow cytometry to determine bulk average fluorescence. Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001). FIG. 3D. Representative histograms of EYFP+/−ABI conditions in FIG. 3C. FIG. 3E. Active loading of Cas9-ABI with and without an NLS into EVs. 6.0×10⁸ EVs were loaded per lane. Expected band sizes (˜160 or 195 kDa, arrows) correspond to Cas9−/+ the ABI domain. The full blot is provided in FIG. 19D. FIG. 3F. Analysis of ABA-dependent Cas9-ABI loading into EVs enriched for anti-CD2 scFv-PYL via affinity chromatography. 1.3×10⁷ MVs or 2.0×10⁷ exosomes were loaded per lane. Expected band size: 195 kDa (arrows). Full blots are provided in FIG. 20B. FIG. 3G. Bioactivity of EV-associated Cas9. Vesicles were lysed and incubated with a linearized target plasmid for 1 h at 37° C. in Cas9 nuclease reaction buffer. Expected cut band sizes: 7.6 and 4.6 kb (arrows).

FIGS. 4A-4F show that viral glycoprotein display on EVs mediates uptake by recipient T cells. FIG. 4A. Illustration of viral glycoproteins facilitating EV uptake and fusion at either the plasma membrane or in the endosome. FIG. 4B. Uptake of dTomato-labeled VSV-G EVs by Jurkat T cells. FIG. 4C. Uptake of dTomato-labeled VSV-G EVs by primary human CD4⁺ T cells. FIG. 4D. Surface expression of SLAM on T cells. Unmodified Jurkats, Jurkats expressing transgenic SLAM, or primary human CD4⁺ T cells were evaluated for SLAM surface expression by flow cytometry. FIG. 4E. Uptake of dTomato-labeled measles viral glycoproteins H/F EVs by Jurkats (+/−SLAM). FIG. 4F. Uptake of dTomato-labeled measles virus glycoproteins H/F EVs by primary human CD4⁺ T cells. In all cases, EVs were incubated with cells for 16 h and trypsinized to remove surface-bound vesicles. Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001). EV dTomato loading evaluations are in FIGS. 21A-21E.

FIGS. 5A-5C show that EVs mediate functional delivery of Cas9-gRNA in primary human T cells. FIG. 5A. Illustration of function delivery evaluation. 2.0×10¹⁰ EVs were incubated with 5.0×10⁴ CD4⁺ T cells for 6 d prior to genomic DNA extraction and HTS analysis. FIG. 5B (SEQ ID NOs: 48 and 49, respectively, in order of appearance). Frequency of indels detected at the Cas9-targeted CXCR4 locus. The sgRNA recognition site (green), PAM sequence (underlined, red), and predicted cut site (amplicon position 26, scissors) are shown. Total percentage of HTS reads classified as “edited” represents the area under the histogram trace shown for each sample. FIG. 5C. Distributions of EV-Cas9-mediated edits, by type. DNA amplicon position is plotted on the abscissa and length of the edit observed is plotted on the ordinate, while the size of each dot scales with the number of edits that meet that description. Each read is uniquely classified as a deletion, insertion, or substitution such that no one read contributes to more than one dot in this panel. In the case of substitutions, the positive ordinate reports the insertion portion of the edit, and the negative ordinate reports the deletion portion of the edit, such that in this case each edit corresponds to two dots. In this panel, deletions are reported by placing a dot at the midpoint of the deleted segment. To help explain the apparent “V” pattern, dots are colored blue to indicate cases where one end of the deleted segment corresponds to the predicted cut region, presumably corresponding to a subset of the DNA repair outcomes observed. Sample dot coloring is as in FIG. 5B.

FIGS. 6A-6F show that CD2 engagement and repeat dosing enhance EV-mediated functional cargo delivery and vary with vesicle subpopulation. FIG. 6A. Illustration of strategy for probing the requirement of scFv-CD2 engagement by blocking CD2. FIG. 6B. Blocking CD2 on recipient cells prior to EV addition increases total editing for all vesicle types. 8.0×10⁹ EVs were incubated per 4.0×10⁴ CD4⁺ T cells for 6 d prior to genomic DNA extraction and HTS analysis. Heat map coloring scales from 0-6% total Cas9-mediated editing. FIGS. 6C-6D. Illustration (FIG. 6C) and evaluation (FIG. 6D) of experiments probing Cas9-mediated editing after repeat EV administration and various modes of CD2 engagement. Two independent experiments using different donor cells and EV preparations are shown. EV dosing was: Donor 1-1.25×10¹⁰ MVs or 5.50×10⁹ exos per 5×10⁴ cells; Donor 2-1.50×10¹⁰ MVs or 7.50×10⁹ exos per 5×10⁴ cells. Heat map coloring is as in FIG. 6B. FIG. 6E. EV-mediated Cas9 functional delivery shows consistent trends across 3 donors and EV batches. Editing efficiency was normalized to the sample receiving VSV-G exosomes (open bar) for each of three independent experiments. FIG. 6F. Combined analysis of experiments presented in FIG. 6D. Within each vesicle population, editing efficiencies were normalized to the sample receiving multiple doses of VSV-G EVs (open bars); this normalization strategy is designed to control for expected sources of greatest variation (i.e., intrinsic donor/T cell batch-specific susceptibility to EVs and editing). Error bars represent one standard deviation.

FIGS. 7A-7C show that EVs harvested from anti-CD2 scFv-expressing cell lines contain full length scFvs. FIG. 7A. Surface stain (via 3×FLAG tag) of stable cell lines expressing anti-CD2 scFvs with different linkers between the binding and transmembrane domains. FIGS. 7B-7C. Expression of scFvs in stable HEK293FT producer cell lines (FIG. 7B) or EVs harvested from those cell lines (FIG. 7C). Expected band size: ˜38-40 kDa (arrows). 0.5 μg cell lysate or 1.0×10⁸ EVs were loaded per lane.

FIGS. 8A-8C show that EVs harvested via differential ultracentrifugation display characteristic surface markers, size distribution, and morphology. FIG. 8A. Detection of CD9 (25 kDa), CD81 (26 kDa), and Alix (96 kDa) in both microvesicle (MV) and exosome (Exo) EV fractions. EV fractions contained minimal calnexin (˜90 kDa). Expected band positions are indicated by arrows. 3 pg cell lysate or 4.5×10⁸ vesicles were loaded per lane. FIG. 8B. Representative NTA size distributions of EV subpopulations. Numbers above histograms refer to the mode size. Error bars (black) indicate standard error of the mean, calculated for each bin. FIG. 8C. Representative TEM of EV subpopulations.

FIG. 9 shows that Jurkat T cells express CD2 on the cell surface. Cells were surface stained for CD2 expression and analyzed by flow cytometry.

FIG. 10 shows that repeat washing removes non-specifically bound EVs from Jurkats. EVs loaded with dTomato were incubated with Jurkat T cells for 2 h at 37° C. and subjected to different numbers of washes to remove excess vesicles prior to analysis by flow cytometry. Cells treated with trypsin for 5 min after EV incubation were used as a reference (a proxy for complete EV removal from the cell surface). 3 washes were used in all subsequent binding assays.

FIGS. 11A-11H shows that EVs harvested from fluorescent cells have similar mean fluorescence within subsets. FIG. 11A. EV dTomato loading evaluations for FIG. 2B. EVs were adsorbed to aldehyde/sulfate latex beads and analyzed by flow cytometry to determine a bulk population fluorescence. Experiments were performed in biological triplicate, and error bars indicate standard error of the mean. FIG. 11B. Representative histograms of EV-loaded bead fluorescence distributions represented in FIG. 11A. FIGS. 11C-11H. EV dTomato loading evaluations for FIG. 2D and FIG. 12 (FIG. 11C), FIG. 2E (FIG. 11D), FIG. 2F (FIG. 11E), FIGS. 2G-2H (FIG. 11F), FIG. 15D (FIG. 11G), and FIG. 15F (FIG. 11H).

FIG. 12 shows that blocking EV recipient cells with blank EVs does not impact targeted or background binding. Recipient Jurkat T cells were incubated for 1 h in the presence or absence of non-fluorescent, non-targeted EVs (red) to block scavenger receptors prior to a 2 h incubation with fluorescent, targeted vesicles (purple). Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001). EV dTomato loading evaluations are presented in FIG. 11C.

FIGS. 13A-13E show that codon optimization increases scFv display on EVs without altering EV morphology. FIG. 13A. Expression of scFv constructs in EV producer cell lysates with different levels of codon optimization. The low-expressing (non-optimized) construct was used in previous experiments, the medium-expressing construct was generated through manual codon optimization, and the high-expressing construct was optimized through Fisher GeneArt synthesis. The low- and high-expressing constructs were carried forward for further evaluation. 0.2 μg cell lysate was loaded per lane. Expected band size: ˜40 kDa (arrows). FIG. 13B. Expression of original and optimized scFv constructs in stable EV producer cell lines. 2 μg cell lysate was loaded per lane. FIG. 13C. scFv display in EVs harvested from cells in FIG. 13B. 4.5×10⁸ EVs were loaded per lane. FIG. 13D. Representative NTA size distributions of optimized scFv-displaying EV subpopulations. Numbers above histograms refer to the mode size. Error bars (black) indicate standard error of the mean, calculated for each bin. FIG. 13E. Representative TEM of optimized scFv-displaying EVs.

FIG. 14 shows that primary T cells express CD2 on the cell surface. Cells were surface stained for CD2 expression and analyzed by flow cytometry.

FIGS. 15A-15G show that different scFv display techniques result in different EV targeting properties. FIG. 15A. Cartoon highlighting the structures of the PDGFR transmembrane domain scFv display and lactadherin C1C2 domain anchoring to phosphatidylserine. FIG. 15B. Expression of scFv constructs in EV producer cell lysates. 1 μg cell lysate was loaded per lane. Expected band sizes: ˜40 kDa and ˜75 kDa (black arrows). FIG. 15C. Loading of scFv constructs into EVs generated from cell lines in FIG. 15B. 5.0×10⁸ EVs were loaded per lane. FIG. 15D. Binding of targeted EVs to Jurkat T cells following a 2 h incubation. FIG. 15E. Representative histograms corresponding to the summary data reported in FIG. 15D. The subpopulation of cells showing a skewed, high degree of exosome binding is indicated by the red box. FIG. 15F. Recipient Jurkat T cells were incubated for 1 h in the presence or absence of anti-CD2 antibodies prior to a 2 h incubation with EVs. FIG. 15G. Representative histograms corresponding to the summary data reported in FIG. 15F. Flow cytometry experiments were performed in biological triplicate, and error bars (panels FIGS. 15D-15F) indicate standard error of the mean. EV dTomato loading evaluations are presented in FIGS. 11A-11H. Statistical tests comprise two-tailed Student's t-tests using the Benjamini-Hochberg method to reduce the false discovery rate (*p<0.05, **p<0.01, ***p<0.001).

FIGS. 16A-16C shows that ABA-binding domains can be incorporated into EV cargo proteins. FIG. 16A. Expression of EYFP fused to the ABI and PYL ABA-binding domains with and without an NLS in transiently transfected HEK293FT cells analyzed by flow cytometry. Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001). FIG. 16B. Surface stain (via 3×FLAG tag) of HEK293FTs transfected with anti-CD2 scFv constructs fused to ABI or PYL at the C-terminus. FIG. 16C. Expression of anti-CD2 scFv constructs from FIG. 16B. 2 μg cell lysate was loaded per lane. Expected band sizes: ˜40, 62, and 75 kDa (arrows).

FIGS. 17A-17B show that ABA induces dimerization between the ABI and PYL domains. FIGS. 17A-17B HEK293FT cells transfected with anti-CD2 scFv-PYL and EYFP-ABI were treated with EtOH (FIG. 17A) or ABA (FIG. 17B) and imaged via confocal microscopy. Brightfield, fluorescence, contrast-adjusted and pseudo-colored fluorescence, and overlays are shown.

FIGS. 18A-18C show that the ABI domain increases EV cargo loading independent of total protein expression. FIG. 18A. Expression of EYFP and EYFP-ABI in the presence of anti-CD2 targeting constructs in transiently transfected HEK293FT cells was analyzed by flow cytometry. A key observation is that addition of the ABI domain does not increase overall cargo protein expression in producer cells. FIG. 18B. Repeat of EYFP-ABI EV loading trends in the presence of an scFv shown in FIG. 3C. FIG. 18C. Comparison of EYFP loading into EVs with and without an NLS with ABA-binding constructs and under ABA-induced dimerization conditions. Addition of an NLS did not substantially impact EYFP loading, nor did ABA-induced dimerization substantially impact loading of nuclear-localized cargo. Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001).

FIGS. 19A-19E show that the ABI domain increases Cas9 loading into EVs and Cas9-ABI retains function. FIG. 19A. Expression of Cas9 fused to either the ABI or PYL domain in transiently transfected HEK293FT cells. 2 μg cell lysate was loaded per lane. Expected band sizes: ˜160, 183, and 195 kDa (arrows). FIG. 19B. Cartoon illustrating the Cas9 reporter construct. Successful editing by Cas9 results in the deletion of a stop codon and (in some random fraction of cases) a repair-mediated frame shift induces express dTomato. FIG. 19C. Absence of an NLS or presence of the ABI domain does not meaningfully reduce Cas9 editing efficiency in transiently transfected Jurkat T cells. Cells were analyzed by flow cytometry 3 d post-transfection. Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001). Samples with high cellular autofluorescence were excluded from analysis. FIG. 19D. Full blot of Cas9 EV active loading data presented in FIG. 3E. FIG. 19E. Cellular expression of Cas9 with and without the ABI domain or an NLS. 2 μg cell lysate was loaded per lane.

FIGS. 20A-20B show that EVs populations can be separated by affinity chromatography to analyze cargo loading patterns. FIG. 20A. Validation of affinity chromatography technique. 3×FLAG tagged scFv containing vesicles were run through an anti-FLAG affinity matrix and analyzed for the FLAG tag to demonstrate enrichment in the eluted population. 1.5×10⁷ EVs were loaded per lane. Expected band size: ˜62 kDa (arrow). FIG. 20B. Full blots of affinity-isolated EV Cas9 content with and without ABA-induced dimerization presented in FIG. 3F.

FIGS. 21A-21E show EV dTomato loading evaluations for vesicle uptake and fusion experiments. FIGS. 21A-21E. EV fluorescence controls for FIG. 4B (FIG. 21A), FIG. 4C (FIG. 21B), FIG. 4E (FIG. 21C), FIG. 4F (FIG. 21D), and FIG. 22 (FIG. 21E). EVs were adsorbed to aldehyde/sulfate latex beads and analyzed by flow cytometry to determine a bulk population fluorescence. Experiments were performed in biological triplicate, and error bars indicate standard error of the mean.

FIG. 22 shows that display of Cx43 on EVs does not lead to increased EV uptake by Jurkat T cells. dTomato EVs were incubated with Jurkats for 16 h. Cells were trypsinized to remove surface-bound vesicles prior to analysis by flow cytometry. Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001). EV dTomato loading evaluations can be found in FIG. 21E.

FIGS. 23A-23D shows that EV-mediated Cas9-gRNA editing results in indels around the predicted CXCR4 cleavage site. FIGS. 23A-23D. Point mutations (FIG. 23A), deletions (FIG. 23B), insertions (FIG. 23C) and substitutions (defined as edits that contain simultaneous insertions and deletions) (FIG. 23D) observed by NGS in primary human CD4⁺ T cells as a function of percent of sequencing reads classified as edited. Each edit observed was classified uniquely into one of these four categories. Predicted Cas9 cut site was position 26 of the amplicon shown in FIGS. 5A-5C.

FIG. 24 shows that presence of the ABI active loading domain does not generally impact Cas9 editing efficiency. 8.0×10⁹ EVs were incubated per 4×10⁴ CD4⁺ T cells for 6 d prior to genomic DNA extraction and NGS analysis. Heat map coloring scales from 0-0.3% total Cas9 editing.

FIG. 25 shows that CD2 engagement does not affect primary T cell activation state. Primary human CD4⁺ T cells were stained with anti-CD25 at the time of EV or anti-CD2 antibody treatment (upper) or 2 d post-treatment (lower). Unstimulated cells were used as a control for background anti-CD25 staining, and isotype controls were used to determine the impact of treatments on general cellular staining. Fluorescence was normalized using calibration beads to allow for signal comparison across days. MEPE: Entities of equivalent PE.

FIGS. 26A-26B show representative flow cytometry gating strategy for EV delivery experiments. FIG. 26A. Live cells were identified based on their FSC-A vs SSC-A profile, and singlets were identified from live cells by their FSC-A vs FSC-H profile. Mean fluorescence intensity was quantified from singlets. FIG. 26B. Aldehyde/sulfate latex beads were identified based on their FSC-A vs SSC-A profile. Mean fluorescence intensity was quantified from beads.

FIG. 27 shows an overview of the plasmids used in Example 18. A first plasmid encodes the lentiviral helper genes: gag, pol, rev, and tat in which expression is driven by the cytomegalovirus promoter (pCMV). A second plasmid encodes the lentiviral backbone including a 5′ long terminal repeat (LTR) and 3′ LTR in which the U3 promoter sequence has been disrupted by deletion to yield a self-inactivating lentiviral vector. The lentiviral payload is driven by the human elongation factor 1a promoter (phEF1a) and comprises the mNeonGreen fluorescent protein. The 3′ portion of the lentiviral genome includes a woodchuck hepatitis virus post-transcriptional regulatory element (WPRE) to stabilize both lentiviral genomic RNA and payload encoding mRNA driven from this vector. A third plasmid encodes a fusogen (driven by the pCMV promoter); fusogen genes included in this vector include the wild-type vesicular stomatitis virus glycoprotein, VSV-G (VSVGwt), or a mutant version of VSV-G that cannot bind its cognate receptor (VSVGmut), or a non-coding sequence (blank). A fourth plasmid encodes a membrane-displayed targeting chimeric polypeptide (here designated “binder”); binder genes included in this vector include a single chain variable fragment of an antibody which binds CD2 (anti-CD2 scFv), or a variable lymphocyte receptor binding domain which binds to CD5 (anti-CD5 VLR).

FIGS. 28A-28B show transduction of Jurkat T cells with extracellular vesicles comprising a lentivirus core (EV-LV) displaying wild type (wt) VSV-G as a fusogen entity in combination with a targeting domain selected from anti-CD2 and anti-CD5. Four EV-LV doses of 1 ul, 2.5 ul, 10 ul, 25 ul and 100 ul with or without polybrene were tested. B shows transduction of Jurkat T cells evaluated under similar conditions to A, but in this case with doses reported in viral genomes (quantified by qPCR) added per recipient Jurkat T cell.

FIGS. 29A-29C show transduction of Jurkat T cells with EV-LV displaying wild type (wt) VSV-G and A no targeting domain; B Anti-CD2; or C Anti-CD5 VLR; as based on flow-based fluorescent readout. These histograms correspond to the viral doses and samples reported in FIG. 28B.

FIGS. 30A-3-B: FIG. 30A shows transduction of Jurkat T cells with EV-LV displaying mutated (mut) VSV-G as a fusogen entity in combination with a targeting domain selected from anti-CD2 and anti-CD5. Four EV-LV doses of 1 ul, 2.5 ul, 10 ul, 25 ul and 100 ul with or without polybrene were tested. FIG. 30B shows transduction of Jurkat T cells evaluated under similar conditions to FIG. 30A, but in this case with doses reported in viral genomes (quantified by qPCR) added per recipient Jurkat T cell.

FIGS. 31A-31C show transduction of Jurkat T cells with EV-LV displaying mutated (mut) VSV-G and FIG. 31A no targeting domain; FIG. 31B Anti-CD2; or FIG. 31C Anti-CD5 VLR; as based on flow-based fluorescent readout. These histograms correspond to the viral doses and samples reported in FIG. 30B.

FIGS. 32A-32B: FIG. 32A shows transduction of Jurkat T cells with EV-LV displaying mutated (mut) VSV-G and the indicated affinity reagents; this figure replots the highest EV-LV dose conditions from FIGS. 31A-31C for comparison. FIG. 32B shows transduction of Jurkat T cells with bilayer lipid particles that display a targeting domain only. The bilayer lipid particles not display a fusogen. This panel replots the relevant conditions from FIGS. 31A-31C for comparison.

FIGS. 33A-33I show transduction of HEK293FT cells with EV-LV. In each case, the EV-LV were formulated such that their surfaces include neither a fusogen nor a targeting chimeric polypeptide (FIG. 33A), only a targeting chimeric polypeptide (FIG. 33D, FIG. 33G), only a fusogen (FIG. 33B, FIG. 33C), or combinations of fusogens and targeting chimeric polypeptides (FIG. 33E, FIG. 33F, FIG. 33H, FIG. 33I).

FIG. 34 shows an overview of the plasmids used in Example 19. A first plasmid encodes the lentiviral helper genes: gag, pol, rev, and tat in which expression is driven by the cytomegalovirus promoter (pCMV). A second plasmid encodes the lentiviral backbone including a 5′ long terminal repeat (LTR) and 3′ LTR in which the U3 promoter sequence has been disrupted by deletion to yield a self-inactivating lentiviral vector. The lentiviral payload is driven by the human elongation factor 1a promoter (phEF1a) and comprises the miRFP720 fluorescent protein. The 3′ portion of the lentiviral genome includes a woodchuck hepatitis virus post-transcriptional regulatory element (WPRE) to stabilize both lentiviral genomic RNA and payload encoding mRNA driven from this vector. A third plasmid encodes a fusogen (driven by the pCMV promoter); fusogen genes included in this vector include the wild-type vesicular stomatitis virus glycoprotein, VSV-G (VSVGwt), or a mutant version of VSV-G that cannot bind its cognate receptor (VSVGmut). A fourth plasmid encodes a targeting chimeric polypeptide (here designated “binder”); binder genes included in this vector include a variable lymphocyte receptor binding domain which binds to CD5 (anti-CD5 VLR).

FIGS. 35A-35B: FIG. 35A shows transduction of activated human CD4⁺ T cells with EV-LV displaying wild type (wt) VSV-G or mutated (mut) VSV-G as a fusogen entity in combination with an anti-CD5 targeting chimeric polypeptide and FIG. 35B shows transduction of activated human CD8⁺ T cells with EV-LV displaying wild type (wt) VSV-G or mutated (mut) VSV-G as a fusogen entity in combination with an anti-CD5 targeting chimeric polypeptide. Reproducibility across lentiviral backbones is shown using two different backbones which differ only in backbone sequence (backbone version 1 or 2, as indicated).

FIG. 36 shows transduction of activated human CD4⁺ T cells with EV-LV displaying wild type (wt) VSV-G or mutated (mut) VSV-G as a fusogen entity in combination with an anti-CD5 target chimeric polypeptide. These histograms correspond to the viral doses and samples reported in FIG. 35A. Reproducibility across lentiviral backbones is shown using two different backbones which differ only in backbone sequence (backbone version 1 or 2, as indicated).

FIG. 37 shows transduction of activated human CD8⁺ T cells with EV-LV displaying wild type (wt) VSV-G or mutated (mut) VSV-G as a fusogen entity in combination with an anti-CD5 targeting domain. These histograms correspond to the viral doses and samples reported in FIG. 35B. Reproducibility across lentiviral backbones is shown using two different backbones (ver and ver7, indicated as suffices).

FIG. 38 shows transduction of HEK293FT cells with EV-LV displaying wild type (wt) VSV-G or mutated (mut) VSV-G as a fusogen entity in combination with an anti-CD5 targeting domain. Reproducibility across lentiviral backbones is shown using two different backbones which differ only in backbone sequence (backbone version 1 or 2, as indicated).

### DETAILED DESCRIPTION

The present disclosure provides surprising insights and useful technologies for delivery of cargo entities to recipient cells of interest. Among other things, the present disclosure appreciates that extracellular vesicles displaying fusion proteins comprising a targeting domain and a transmembrane domain (e.g., a PDGFR transmembrane domain) demonstrate particularly efficient and/or effective vesicle targeting to specific recipient cells. Exemplary useful such fusion proteins are described in PCT publication WO2019/199941 (entitled “Extracellular vesicles comprising targeting affinity domain-based membrane proteins”, and published 17 Oct. 2019, the content which is incorporated herein by reference in its entirety), which demonstrates that such fusion proteins, when displayed on various vesicle types (e.g., exosomes, microvesicles), can mediate vesicle uptake by recipient cells. The present disclosure provides a surprising further development relative to such fusion proteins, demonstrating, for example, that their combination with fusogen entity polypeptides as described herein on various lipid bilayer particles can achieve remarkably efficient and/or effective targeted delivery of cargo to particular recipient cells of interest, including to particular human and/or immune cells, and specifically to T cells (e.g., human T cells), thereby providing unique and important value in the field.

Those skilled in the art, reading the present specification, will appreciate that when cargo entities are delivered to recipient cells in accordance with the present teachings, such cargo entities in some embodiments may modify (e.g., genetically modify) recipient cell(s), as is useful in a number of applications including various therapeutic applications. Cargo entities may be delivered by lipid bilayer particles, including cell-derived membrane particles (CDMPs) (e.g., extracellular vesicles (EVs)) to particular recipient cells.

As noted herein, particularly useful embodiments of provided technologies achieve genetic modification of T cells. Genetically modifying T cells can enable applications ranging from cancer immunotherapy to HIV treatment, yet delivery of T cell-targeted therapeutics remains challenging. Extracellular vesicles (EVs) and other cell-derived membrane particles (CDMPs) are nanoscale particles secreted by all cells that naturally encapsulate and transfer proteins, nucleic acids, and nucleic acid—protein complexes, such as viral nucleocapsids (e.g., enveloped viruses) making them an attractive and clinically-relevant platform for engineering biocompatible delivery vehicles. The present disclosure provides a suite of technologies for genetically engineering production cells to produce multifunctional lipid bilayer particles (e.g., CDMPs such as EV)—without employing chemical modifications that complicate biomanufacturing. The present disclosure further provides high affinity targeting domains (e.g., targeting chimeric polypeptides) and/or fusogen entities that are displayed on lipid bilayer particle surfaces to achieve specific, efficient binding to recipient cells (e.g., immune cells, such as T cells). Fusogen entity polypeptides (e.g., glycoproteins) may increase lipid bilayer particle uptake and fusion with recipient cells. The present disclosure also identify a protein tag to confer active cargo loading into lipid bilayer particles. The Examples herein demonstrate integration of these technologies by delivering Cas9-sgRNA complexes to edit primary human T cells, viral nucleocapsid derivatives (e.g. lentivirus nucleocapsid), or fluorescent proteins to validate fusion of lipid vesicles to recipient cells. These approaches could enable targeting particles to a range of cells for the efficient delivery of cargo.

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001)Molecular Cloning: A Laboratory Manual, 3rd edition; the series Ausubel et al., eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al., eds (1996) Weir's Handbook of Experimental Immunology.

### Definitions

Unless otherwise specified or indicated by context, the terms “a”, “an”, and “the” mean “one or more.” For example, “a fusion protein,” “an extracellular vesicle,” and “a cell” should be interpreted to mean “one or more fusion proteins,” “one or more extracellular vesicles,” and “one or more cells,” respectively.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold or that has a known affinity below a particular threshold. In some embodiments, affinity may be assessed relative to a contemporaneous reference; in some embodiments, affinity may be assessed relative to a historical reference. Typically, when affinity is assessed relative to a reference, it is assessed under comparable conditions.

As used herein “binding moiety” is used herein to refer to a moiety that binds to a target ligand of interest as described herein (e.g., a target ligand on recipient cell surface(s), or populations thereof). In many embodiments, a binding moiety of interest is one that binds specifically with its target ligand in that it discriminates its target ligand from other potential binding partners in a particular interaction context. In some embodiments, a binding moiety shows specific binding to its target ligand relative to one or more other entities on the surface of recipient cell(s). Alternatively or additionally, in some embodiments, a binding moiety shows preferential binding to its target ligand relative to one or more (or all) entities present on surfaces of non-recipient cell(s) (e.g., non-recipient cell(s) that may be present in a system that includes recipient cells). In some embodiments, a binding moiety binds one or more target ligands and drives a specific biological activity that is only linked to a specific target ligand. In some embodiments, a binding moiety is a peptide binding moiety. In some embodiments, a binding moiety is a non-peptide binding agent. In some such embodiments, a production cell may be engineered to express a targeting chimeric polypeptide comprising a binding moiety that is subsequently modified (e.g., chemically modify) by attaching a non-polypeptide binding moiety, so that the non-polypeptide binding moiety provides specific affinity to a target ligand. In some embodiments, a binding agent comprises (i) a targeting chimeric polypeptide, and optionally a non-polypeptide portion, and (ii) when present on surfaces of lipid bilayer particles binds to target cells. In general, a binding moiety may be or comprise a moiety of any chemical class (e.g., polymer, non-polymer, small molecule, polypeptide, carbohydrate, lipid, nucleic acid, etc). In some embodiments, a binding moiety is a single chemical entity. In some embodiments, a binding moiety is a complex of two or more discrete chemical entities associated with one another under relevant conditions by non-covalent interactions. For example, those skilled in the art will appreciate that in some embodiments, a binding moiety may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple affinity moieties through linkage of different specific binding moieties with the same generic binding moiety partner. In some embodiments, binding moieties are or comprise polypeptides (including, e.g., antibodies or antibody fragments). In some embodiments, binding moieties are or comprise small molecules. In some embodiments, binding moieties are or comprise nucleic acids. In some embodiments, binding moieties are aptamers. In some embodiments, binding moieties are polymers; in some embodiments, affinity moieties are not polymers. In some embodiments, binding moieties are non-polymeric in that they lack polymeric moieties. In some embodiments, binding moieties are or comprise carbohydrates. In some embodiments, binding moieties are or comprise peptidomimetics. In some embodiments, binding moieties are or comprise scaffold proteins. In some embodiments, binding moieties are or comprise mimeotopes. In some embodiments, binding moieties are or comprise stapled peptides. In certain embodiments, binding moieties are or comprise nucleic acids, such as DNA or RNA.

As used herein, the phrase “characteristic sequence element” refers to a sequence element found in a polymer (e.g., in a polypeptide or nucleic acid) that represents a characteristic portion of that polymer. Those skilled in the art appreciate that, presence of a characteristic sequence element typically correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

As used herein, “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

As used herein, a “control” is an alternative sample used in an experiment for comparison purpose. A control can be “positive” or “negative.” For example, where the purpose of the experiment is to determine a correlation of the efficacy of cargo protein loading to EVs and the structures of the cargo proteins, a positive control (a cargo protein known to exhibit the desired loading efficacy) and a negative control (a cargo protein that does not load to EVs) are typically employed.

As used herein “corresponding to” refers to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

As used herein, the term “engineered” refers to the aspect of having been designed, produced, and/or manipulated by the hand of man. For example, a polynucleotide is considered to be “engineered” when two or more sequences that are not linked together in that order in nature are designed or otherwise caused by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, in some embodiments described and/or utilized herein, an engineered polynucleotide comprises a regulatory sequence that is found in nature in operative association with a first coding sequence but not in operative association with a second coding sequence, is linked by the hand of man so that it is operatively associated with the second coding sequence. Comparably, in some embodiments a polypeptide may be considered to be “engineered” if encoded by or expressed from an engineered polynucleotide, and/or if produced other than natural expression in a cell. Analogously, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

As used herein “engineered lipid bilayer particles” refers to a lipid bilayer particle engineered as described herein. For example, in some embodiments, a lipid bilayer particle may be considered to be “engineered” if it is synthetically produced, i.e., not produced by a cell. Alternatively or additionally, in some embodiments, a lipid bilayer particle may be considered to be “engineered” if it is produced by an engineered production cell. In some embodiments, an engineered lipid bilayer particle is produced by a production cell engineered to have a fusogen entity polypeptide and/or targeting chimeric polypeptide on its surface. In some such embodiments, an engineered production cell differs from an appropriate reference cell in that it has been engineered to express a fusogen entity polypeptide, a targeting chimeric polypeptide, or both, or to express one or both at a different level (e.g., an elevated level) such that lipid bilayer particles (e.g., CDMPs) produced (e.g., released) by such engineered production cell bind to a recipient cell, or population of cells, with significantly greater affinity and/or specificity than do comparable particles produced (e.g., released) by the reference cell.

As used herein, the term “extracellular vesicles” should be interpreted to include all nanometer-scale lipid vesicles that are secreted and/or budding by cells such as exosomes and microvesicles, respectively. As used herein, the term “exosomes” refer to extracellular vesicles originate from internal endocytic compartments and multi-vesicular bodies, and the term “microvesicles” refer to vesicles that bud directly from the cell surface. EVs, and their isolation and analysis are well-known to a skilled in the art. See, for example, Doyle et al., Cells 8(7): 727 (2019), which is incorporated herein by reference in its entirety. Extracellular vesicles may be taken up by so-called extracellular vesicle (EV) recipient cells. As utilized herein, the term “recipient cell” may be interchangeably with the term “target cell.”

As used herein, the term “cell-derived membrane particle” should be interpreted to include any membrane-derived vesicles or particle that can be generated by blebbing or budding, and can include hybrid vesicles generated by mixing vesicles that were generated from cells and synthetic vesicles, as well as vesicles or particles generated by mechanically processing cells. Thus, “cell-derived membrane particles” can include, but is not limited to, extracellular vesicles (as defined above), virus particles, virus-like particles (VLPs), apoptotic bodies, and platelet-like particles.

As used herein term “fusogen entity polypeptide” refers to a polypeptide that mediates fusion between lipid bilayers. As documented by the present disclosure, in some embodiments, presence of a fusogen entity polypeptide in or engineered lipid bilayer particles (including specifically on engineered lipid bilayer particle displaying a targeting chimeric polypeptide) increases efficiency, specificity and/or effectiveness of cargo delivery from such engineered lipid bilayer particles to particular recipient cells of interest.

As used herein, the term “gene” means a segment of DNA that contains information for the regulated biosynthesis of an RNA product. Those skilled in the art will appreciate that a gene typically includes an expressed sequence (e.g., an open reading frame which may for example include exons and introns. A skilled person will further appreciate that a gene typically includes one or more promoters and/or other untranslated regions (e.g., enhancer elements, repressor elements, chromatin binding sites, etc.), that may control, regulate, or otherwise impact expression.

As used herein, “homology” or “identity” or “similarity” refers to sequence similarity between two peptides or between two nucleic acid entities. Those skilled in the art appreciate that homology can be determined by comparing a position in each sequence which may be aligned for purposes of comparison. When a position in the compared sequence is occupied by the same base or amino acid, then the entities are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other. Furthermore, those skilled in the art will appreciate that “homologous” polypeptides or nucleic acids may often share one or more characteristic sequence elements, e.g., that may impart a shared structural and/or functional feature to polypeptides that include it.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “linker” refers that portion of a multi-element agent that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker. In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. In some embodiments, a linker is characterized in that it adopt a rigid three-dimensional structure and provides a stability to the polypeptide. A variety of different linker elements that can appropriately be used when engineering polypeptides (e.g., fusion polypeptides) known in the art (see e.g., Holliger, P., et al. (1993) Proc. Natl. Acad. Sci. USA 90:6444-6448; Poljak, R. J., et al. (1994) Structure 2: 1 121-1123).

As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. As will be clear from context, these phrases can also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences,” FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99%, or 100% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. Coli, plants, and other host cells.

Regarding polynucleotide sequences, a “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

“Transformation” or “transfected” describes a process by which exogenous nucleic acid (e.g., DNA or RNA) is introduced into a recipient cell. Transformation or transfection may occur under natural or artificial conditions according to various methods well-known in the art and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g., in vitro or ex vivo administration) or target tissues (e.g., in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time. In another embodiment, the term also includes stably transfected cells.

The polynucleotide sequences contemplated herein may be present in expression vectors. For example, the vectors may comprise: (a) a polynucleotide encoding an ORF of a cargo protein; and (b) a polynucleotide that expresses an ABA-binding domain, e.g., a pyrabactin resistance 1-like (PYL1) sequence or an abscisic acid-insensitive 1 (ABI1) sequence. The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Expression vectors as contemplated herein may include eukaryotic or prokaryotic control sequences that modulate expression of a heterologous protein (e.g., the fusion protein disclosed herein). Prokaryotic expression control sequences may include constitutive or inducible promoters (e.g., T3, T7, Lac, trp, or phoA), ribosome binding sites, or transcription terminators.

The vectors contemplated herein may be introduced and propagated in a prokaryote, which may be used to amplify copies of a vector to be introduced into a eukaryotic cell or as an intermediate vector in the production of a vector to be introduced into a eukaryotic cell (e.g. Amplifying a plasmid as part of a viral vector packaging system). A prokaryote may be used to amplify copies of a vector and express one or more nucleic acids, such as to provide a source of one or more proteins for delivery to a host cell or host organism. Expression of proteins in prokaryotes may be performed using Escherichia coli with vectors containing constitutive or inducible promoters directing the expression of either a protein or a fusion protein comprising a protein or a fragment thereof. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; (iii) to aid in the purification of the recombinant protein by acting as a ligand in affinity purification (e.g., a His tag); (iv) to tag the recombinant protein for identification (e.g., such as Green fluorescence protein (GFP) or an antigen (e.g., HA) that can be recognized by a labelled antibody); (v) to promote localization of the recombinant protein to a specific area of the cell (e.g., where the protein is fused (e.g., at its N-terminus or C-terminus) to a nuclear localization signal (NLS) which may include the NLS of SV40, nucleoplasmin, C-myc, M9 domain of hnRNP A1, or a synthetic NLS). The importance of neutral and acidic amino acids in NLS have been studied. (See Makkerh et al. (1996) Curr Biol 6(8):1025-1027). Often, in fusion expression vectors, a proteolytic cleavage site is introduced at the junction of the fusogen moiety and the recombinant protein to enable separation of the recombinant protein from the fusogen moiety subsequent to purification of the fusion protein. Such enzymes, and their cognate recognition sequences, include Factor Xa, thrombin and enterokinase.

The presently disclosed methods may include delivering one or more polynucleotides, such as or one or more vectors as described herein, one or more transcripts thereof, and/or one or proteins transcribed therefrom, to a host cell. Further contemplated are host cells produced by such methods, and organisms (such as animals, plants, or fungi) comprising or produced from such cells. The disclosed extracellular vesicles may be prepared by introducing vectors that express mRNA encoding a fusion protein and a cargo RNA as disclosed herein. Conventional viral and non-viral based gene transfer methods can be used to introduce nucleic acids in mammalian cells or target tissues. Non-viral vector delivery systems include DNA plasmids, RNA (e.g., A transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

In the methods contemplated herein, a host cell may be transiently or non-transiently transfected (i.e., stably transduced) with one or more vectors described herein. In some embodiments, a cell is transfected as it naturally occurs in a subject (i.e., in situ). In some embodiments, a cell that is transfected is taken from a subject (i.e., explanted). In some embodiments, the cell is derived from cells taken from a subject, such as a cell line. Suitable cells may include stem cells (e.g., embryonic stem cells and pluripotent stem cells). A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors, or transfection with RNA), and modified through the activity of a complex, in order to establish a new cell line comprising cells containing the modification but lacking any other exogenous sequence.

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

Regarding proteins, the term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

Regarding proteins, a “deletion” refers to a change in the amino acid sequence that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide; in other embodiments, a fragment may comprise less than about 5, 10, 15, 20, 25, 30, 40, 50, 60, 70, 80, 90, 100, 150, 250, or 500 contiguous amino acid residues of a reference polypeptide; or in other embodiments, a fragment has a length within a range bounded by any of these values (e.g., a range of 50-100 contiguous amino acids of a reference polypeptide). Fragments may be preferentially selected from certain regions of a molecule. The term “at least a fragment” encompasses the full length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues, or a range of amino acid residues bounded by any of these values (e.g., an insertion or addition of 5-10 amino acids). A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, as used herein, “chimeric proteins,” “chimeric peptides,” “fusion proteins,” or “fusion peptides” refer to polypeptides created through the linking two or more functional domains from separate or same proteins via an amino acid linker or directly linked, resulting in a single polypeptide with functional properties derived from each of the original proteins. In some embodiments, a linker is 10-50 amino acids in length and is rich in glycine for flexibility, as well as serine or threonine for solubility. In some embodiments, a linker is characterized in that it adopt a rigid three-dimensional structure and provides a stability to the polypeptide. A “variant” of a reference polypeptide sequence may include a fusion polypeptide comprising the reference polypeptide.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases. As described herein, variants, mutants, or fragments (e.g., a protein variant, mutant, or fragment thereof) may have 99%, 98%, 97%, 96%, 95%, 94%, 93%, 92%, 91%, 90%, 80%, 70%, 60%, 50%, 40%, 30%, or 20% amino acid sequence identity relative to a reference molecule.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original | Conservative |
|---|---|
| Residue | Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, variants, or described herein may have one or more functional or biological activities exhibited by a reference polypeptide (e.g., one or more functional or biological activities exhibited by wild-type protein). For example, the disclosed proteins, mutants, variants, or derivatives thereof may have one or more biological activities that include binding to the small molecule ABA and targeting an EV to a recipient cell.

The disclosed proteins may be substantially isolated or purified. The term “substantially isolated or purified” refers to proteins that are removed from their natural environment, and are at least 60% free, preferably at least 75% free, and more preferably at least 90% free, even more preferably at least 95% free from other components with which they are naturally associated.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “targeting domain” or “targeting peptide” refers to peptide moieties that will facilitate specific binding of the EV to a recipient cell. Sample “targeting domain” or “targeting peptide” include but are not limited to antibodies and any antibody fragments or antigen binding fragments, e.g., Fab, Fab′ and F(ab′)2, Fd, single-chain Fvs (scFv), single-chain antibodies, disulfide-linked Fvs (sdFv), a de novo-designed binding molecule, affinibody, a DARPIN, nanobody, a variable lymphocyte receptor (VLR), and a camelid antibody. Those antibody fragments are well-known to a skilled person in the art.

### Recipient Cells

Among other things, the present disclosure demonstrates surprising effectiveness of provided technologies when applied to recipient cells, e.g. to particular recipient cells of interest, or to population(s) thereof. For example, the present disclosure documents surprising specificity and/or efficiency of cargo entity delivery recipient cells, or populations thereof, of interest, using lipid bilayer particle, such as cell-derived membrane particles (CDMPs) as described herein. Using technologies of the present disclosure lipid bilayer particles deliver a cargo entity to a recipient cell in an efficient and specific manner. In some embodiments, provided technologies delivery cargo entities when employed ex vivo. In some embodiments, provided technologies achieve, e.g., in vivo delivery of a cargo entity to a recipient cell.

In some embodiments, a recipient cell expresses a target ligand (e.g., a cell surface epitope). In some embodiments, a recipient cell expresses a particular target ligand or a specific combination of target ligands. In some embodiments, a recipient cell is CD5+, CD2+, or a combination thereof. In some embodiments, a recipient cell is an immune cell. In some embodiments, an immune cell is a lymphocyte. In some embodiments, a lymphocyte is a T cell. In some embodiments, a T cell is an activated T cell. In some embodiments, a T cell is CD4+ and/or CD8+.

In some embodiments, a targeting chimeric polypeptide provided herein binds a target ligand on the surface of a recipient cell. In some embodiments, a particular targeting chimeric polypeptide (e.g., a targeting chimeric polypeptide on a lipid bilayer particle surface) binds a target ligand on the surface if a recipient cell. In some embodiments, a target ligand (e.g., target epitope) is expressed on all cells (a universal feature of the cell surface), or on a subset of cells, or on cells that occupy a subset of possible states (e.g., activated T cells versus resting T cells).

Without wishing to be bound by any particular theory, it is proposed that the unique combination of a fusogen entity polypeptide and targeting chimeric polypeptide provided herein achieves targeted entry of lipid bilayer particles into specific recipient cells with high efficiency.

In some embodiments, a targeting chimeric polypeptide as described herein binds a specific target ligand on recipient cell surface(s). A recipient cell may express a number of unique targeting ligands on the cell surface and a targeting chimeric polypeptide can be designed to target one or more such target ligands. In some embodiments, a target ligand is on the surface of a recipient cell. In some embodiments, a target ligand is an epitope, receptor, protein, carbohydrate, lipid, or particular combinations or conformational states thereof.

### Engineered Lipid Bilayer Particles

Among other things, the present disclosure provides engineered lipid bilayer particles, and preparations thereof, comprising (e.g., comprising on their surfaces) a targeting chimeric polypeptide, a fusogen entity polypeptide, or a combination thereof as described herein. The present invention provides, in some embodiments, engineered lipid bilayer particles and preparations thereof comprising a targeting chimeric polypeptide and a fusogen entity polypeptide. Provided engineered lipid bilayer particles may be suitable for therapeutic applications.

In some embodiments, the present disclosure provides populations of engineered lipid bilayer particles comprising:
(i) a targeting chimeric polypeptide comprising
   (a) a targeting domain arranged so that the targeting domain is on the particle surface, wherein the targeting domain comprises a binding moiety that specifically binds to a target ligand on surfaces of recipient cells of interest; and linked directly or indirectly with
   (b) a transmembrane domain, and
(ii) a fusogen entity polypeptide comprising
   (a) fusogen moiety arranged so that the fusogen moiety is on the particle surface; linked directly or indirectly with
   (b) a transmembrane domain.

In some embodiments, engineered lipid bilayer particles in a population display one or more targeting chimeric polypeptides and one or more fusogen entity polypeptides. In some embodiments, at least a part of a fusogen entity polypeptide is present on the surface of an engineered lipid bilayer particle (e.g., a fusogen moiety). In some embodiments, at least a part of a targeting chimeric polypeptide is present on the surface of an engineered lipid bilayer particle (e.g., a targeting domain). Without wishing to be bound by any particular theory, it is proposed that the particular combination of a targeting chimeric polypeptide as described herein and a fusogen entity polypeptide provides targeted entry of engineered lipid bilayer particles into specific recipient cells with high efficiency. Indeed, without wishing to be bound by any particular theory, it is proposed that particular targeting chimeric polypeptides as described herein (e.g., utilizing a PDGFR transmembrane domain [or, in some embodiments, a comparable transmembrane domain] and an affinity entity polypeptide, including specifically where such affinity entity polypeptide is or comprises an antibody agent) may provide particular display characteristics (e.g., orientation and/or other aspects of presentation, surface density, etc, that render them particularly amenable to beneficial combination with fusogen entity polypeptides as described herein to achieve particle targeting as described herein.

In some embodiments, engineered lipid bilayer particles are produced by one or more engineered production cells as described herein so that the engineered lipid bilayer particles display a targeting chimeric polypeptide and a fusogen entity polypeptide.

In some embodiments, a population of engineered lipid bilayer particles is characterized in that the engineered lipid bilayer particles are smaller than a eukaryotic cell. In some embodiments, a population of engineered lipid bilayer particles is characterized in that the average diameter of the engineered lipid bilayer particles is at the most 1000 nm, such as at the most 800 nm, such as at the most 300, such as at the most 100 nm. In some embodiments, a population of engineered lipid bilayer particles is characterized in that the average diameter of the engineered lipid bilayer particles is at least 30 nm, such as at least 50 nm, such as at least 80 nm, such as at least 100 nm, such as at least 150 nm, such as at least 200 nm, such as at least 250 nm, such as at least 300 nm. In some embodiments, a population of engineered lipid bilayer particles is characterized in that the average diameter of the engineered lipid bilayer particles is about 10 nm to about 1000 nm, such as about 30 nm to about 800 nm, such as about 50 nm and about 500 nm.

In some embodiments, entry of an engineered lipid bilayer particles into recipient cells may be influenced by one or more attributes of the entry environment (e.g., pH, temperature, proximity-induced, mechanical tension or stress, rearrangement of lipid domains (e.g., rafts), radiation (e.g., nuclear, ultraviolet, visual, etc), electric signal, magnetic field, etc., or a combination hereof).

In some embodiments, an engineered lipid bilayer particle displays at least 5 copies of a fusogen entity polypeptide, such as at least 10 copies, such as at least 50 copies, such as at least 100 copies, such as at least 200 copies, such as at least 300 copies, such as at least 400 copies, such as at least 500 copies of a fusogen entity polypeptide.

In some embodiments, an engineered lipid bilayer particle displays at least 10 copies of an affinity entity polypeptide, such as at least 50 copies, such as at least 100 copies, such as at least 200 copies, such as at least 300 copies, such as at least 400 copies, such as at least 500 copies of an affinity entity polypeptide.

In some embodiments, engineered lipid bilayer particles are cell-derived membrane particles (CDMPs). In some embodiments, CDMPs are selected from extracellular vesicles, virus particles, virus-like particles (VLPs), apoptotic bodies, platelet-like particles, and combinations thereof. In some embodiments, extracellular vesicles are exosomes, microvesicles, and combinations thereof.

In some embodiments, a CDMP is an extracellular vesicle, which can be selected from an exosome or a microvesicle. In some embodiments, a CDMP can be a virus particle, a virus-like particles (VLP), an apoptotic body, and a platelet-like particle. In some embodiments, the CDMP can be a hybrid particle generated by mixing a cell-derived particle or vesicle (e.g., a particle or vesicle that blebbed or budded from a cell) and a synthetic vesicle.

In some embodiments, an engineered lipid bilayer particle displays a fusogen entity polypeptide, such as a VSV-G or a functional variant thereof as described herein. In some embodiments, an engineered lipid bilayer particle displays a targeting chimeric polypeptide comprising a PDGFR transmembrane domain. In some embodiments, an engineered lipid bilayer particle displays a VSV-G as described herein and a targeting chimeric polypeptide that binds to CD2 or CD5.

### Targeting Chimeric Polypeptide

The present disclosure provides insights regarding particularly useful and/or effective targeting chimeric polypeptides to facilitate specific binding of an engineered lipid bilayer particle to a target ligand (e.g., a target ligand present on a recipient cell). The present disclosure teaches that a targeting chimeric polypeptide and its binding to a target ligand can be useful in mediating targeted fusion of a engineered lipid bilayer particle with a recipient cell expressing the target ligand or a particular subset of recipient cells expressing the target ligand (e.g., provide specificity to technologies described herein). Without wishing to be bound by any particular theory, it is proposed that targeted fusion may be driven and/or influenced by one or more of proximity, affinity and/or conformational changes.

In some embodiments, a targeting chimeric polypeptide mediates binding of an engineered lipid bilayer particle to a recipient cell of interest. In some embodiments, a targeting chimeric polypeptide alone (e.g., in absence of a fusogen entity polypeptide) does not promote cell entry and transduction. Moreover, in many embodiments, combination of a targeting chimeric polypeptide as described herein and a fusogen polypeptide as described herein achieves remarkable improvements in efficiency, specificity and/or effectiveness of cargo delivery to particular recipient cells of interest.

Targeting chimeric polypeptides provided herein are useful when designing binding to a specific type of recipient cells. In some embodiments, a targeting chimeric polypeptide binds to a specific target ligand hereby directing binding to recipient cells expressing such specific target ligand. In some embodiments, when a targeting chimeric polypeptide is co-displayed in an engineered lipid bilayer particle with a fusogen entity polypeptide, binding of the targeting chimeric polypeptide promotes fusion of the fusogen entity polypeptide with the recipient cell.

In some embodiments, the present disclosure provides a targeting chimeric polypeptide. In some embodiments, the present disclosure provides a nucleotide sequence that encodes a targeting chimeric polypeptide.

In some embodiments, technologies (e.g., a system, engineered lipid bilayer particle and engineered production cells) according to the present disclosure comprise a targeting chimeric polypeptide. In some embodiments, technologies according to the present disclosure comprise at least one targeting chimeric polypeptide. In some embodiments, technologies according to the present disclosure comprise one or more targeting chimeric polypeptides.

In some embodiments, a targeting chimeric polypeptide comprises a secretory signal. In some embodiments, a targeting chimeric polypeptide comprises a FLAG tag. In some embodiments, a targeting chimeric polypeptide does not comprise a FLAG tag, e.g., when the targeting chimeric polypeptide is a native polypeptide. In some embodiments, a targeting chimeric polypeptide comprises an affinity moiety. In some embodiments, a targeting chimeric polypeptide comprises a linker. In some embodiments, a targeting chimeric polypeptide does not comprise a linker e.g., when the targeting chimeric polypeptide is a native polypeptide. In some embodiments, a targeting chimeric polypeptide comprises a membrane association portion. In some embodiments, a targeting chimeric polypeptide comprises an intraparticle portion.

In some embodiments, a targeting chimeric polypeptide consists or comprises of a transmembrane domain. In some embodiments, a transmembrane domain is a domain that has a hight expression on the surface of a lipid bilayer particle.

In some embodiments, a targeting chimeric polypeptide is an engineered polypeptide. In some embodiments, the order from the N-terminal to the C-terminal of a targeting chimeric polypeptide is as follows: a secretory signal, a targeting domain, a linker, and/or a transmembrane domain. In some embodiments, the order from the N-terminal to the C-terminal of a targeting chimeric polypeptide is as follows: a secretory signal, an FLAG tag, a targeting domain, a linker, and/or a transmembrane domain.

In some embodiments, a targeting chimeric polypeptide is a wild type polypeptide. In some embodiments, a targeting chimeric polypeptide is native to a particular production cell. In some embodiments, a targeting chimeric polypeptide is an engineered polypeptide. In some embodiments, a targeting chimeric polypeptide (e.g., an engineered targeting chimeric polypeptide) is a variant of a wild type polypeptide and/or of a native polypeptide.

In some embodiments, the order from the N-terminal to the C-terminal of a targeting chimeric polypeptide is as follows: a secretory signal, a targeting domain, a transmembrane domain, and/or an intraparticle portion.

In some embodiments, an affinity entity polypeptide includes one or more modifications, such as glycosylation, lipidation, phosphorylation, etc.

In some embodiments, a targeting chimeric polypeptide comprising a targeting domain. In some embodiments, a targeting chimeric polypeptide comprising a targeting domain comprises a binding moiety that specifically binds to a target ligand on surfaces of recipient cells of interest. In some embodiments, a binding domain is displayed on the surface of a lipid bilayer particle. It may be displayed in a way that promotes binding of the lipid bilayer of the lipid bilayer particle with a target ligand on the surface of a recipient cell. In some embodiments, a targeting chimeric polypeptide further comprises a transmembrane domain. In some embodiments, a targeting domain is linked directly or indirectly with a transmembrane domain.

In some embodiments, a targeting domain is or comprises an antibody agent. In some embodiments, an antibody agent is a single chain antibody agent. In some embodiments, an antibody agent is selected from the group consisting of an antibody, a Fab, a Fab′, a F(ab′)2, a Fd, a scFv, a single-chain antibody, a disulfide-linked Fvs (sdFv), an affinibody, a DARPIN, a nanobody, a variable lymphocyte receptor (VLR), and a camelid antibody.

The term “engineered high affinity binding polypeptides” is equivalent to “de novo designed binding molecules” and is used herein interchangeably.

In some embodiments, a binding domain specifically binds to the surface of an immune cell (e.g., a lymphocyte, such as a CD4+ and/or CD8+ T cell). In some embodiments, an affinity moiety is characterized in that it binds to a recipient cell expressing CD5, CD2, or a combination thereof.

In some embodiments, the present disclosure provides chimeric targeting polypeptide comprising: (a) a targeting domain that binds human CD2, wherein the targeting domain is or comprises an antibody agent selected from the group consisting of an antibody, a Fab, a Fab′, a F(ab′)₂, a Fd, a scFv, a single-chain antibody, a disulfide-linked Fvs (sdFv), a de novo-designed binding molecule, an affinibody, a DARPIN, a nanobody, a variable lymphocyte receptor (VLR) and a camelid antibody; and (b) a transmembrane domain. In some embodiments, the targeting domain is a scFv. The chimeric targeting polypeptide may optionally comprise a linker.

In some embodiments, a targeting domain binds to CD2. In some embodiments, a targeting domain comprises an anti-CD2 moiety or a fragment thereof. In some embodiments, a targeting domain comprises the amino acid sequence NIMMTQSPSSLAVSAGEKVTMTCKSSQSVLYSSNQKNYLAWYQQKPGQSPKLLIYWAS TRESGVPDRFTGSGSGTDFTLTISSVQPEDLAVYYCHQYLSSHTFGGGTKLEIKRGGGGS GGGGSGGGGSQLQQPGAELVRPGSSVKLSCKASGYTFTRYWIHWVKQRPIQGLEWIGNI DPSDSETHYNQKFKDKATLTVDKSSGTAYMQLSSLTSEDSAVYYCATEDLYYAMEYW GQGTSVTVSS (SEQ ID NO: 20), which can be encoded by the nucleic acid sequence AACATCATGATGACGCAGAGCCCCAGCAGCCTGGCTGTTTCTGCTGGCGAGAAAGT GACCATGACCTGCAAGAGCAGCCAGAGCGTGCTGTACTCCAGCAACCAGAAGAACT ACCTGGCCTGGTATCAGCAGAAGCCCGGCCAGTCTCCTAAGCTGCTGATCTACTGGG CCAGCACCAGAGAAAGCGGCGTGCCCGATAGATTCACAGGCTCTGGCAGCGGCACC GACTTCACCCTGACAATCAGTAGCGTGCAGCCCGAGGATCTGGCCGTGTACTACTGT CACCAGTACCTGAGCAGCCACACCTTTGGCGGCGGAACAAAGCTGGAAATCAAGAG AGGCGGAGGCGGATCAGGTGGCGGTGGATCTGGCGGTGGTGGATCTCAACTTCAGC AGCCAGGCGCAGAACTTGTGCGGCCTGGATCTAGCGTGAAGCTGAGCTGTAAAGCC AGCGGCTACACCTTCACCAGATACTGGATCCACTGGGTCAAGCAGCGGCCTATCCA GGGACTCGAGTGGATCGGCAATATCGACCCCAGCGACAGCGAGACACACTACAATC AGAAGTTCAAGGACAAGGCCACACTGACCGTGGACAAGTCTAGCGGCACAGCCTAC ATGCAGCTGTCCAGCCTGACAAGCGAGGACAGCGCCGTGTATTATTGCGCCACCGA GGACCTGTACTACGCCATGGAATATTGGGGCCAGGGCACCAGCGTGACCGTTAGCT CT (SEQ ID NO: 21). However, it should be noted that a targeting domain that is capable of binding to CD2 is expected to function. In some embodiments, a chimeric targeting polypeptide and lipid bilayer particle that comprise such polypeptides, CD2 binding is alone sufficient to deliver the contents (e.g., cargo entity) of the lipid bilayer particle into a recipient cell (e.g., lymphocyte).

In some embodiments, the present disclosure provides chimeric targeting polypeptide comprising: (a) a targeting domain that binds human CD5, wherein the targeting domain is or comprises an antibody agent selected from the group consisting of an antibody, a Fab, a Fab′, a F(ab′)₂, a Fd, a scFv, a single-chain antibody, a disulfide-linked Fvs (sdFv), a de novo-designed binding molecule, an affinibody, a DARPIN, a nanobody, a variable lymphocyte receptor (VLR) and a camelid antibody; and (b) a transmembrane domain. In some embodiments, a targeting domain is a VLR. In some embodiments, a targeting domain is a scFv. The chimeric targeting polypeptide may optionally comprise a linker.

In some embodiments, a targeting domain binds to CD5. In some embodiments, a targeting domain comprises an anti-CD5 moiety or a fragment thereof. In some embodiments, the targeting domain comprises the amino acid sequence

(SEQ ID NO: 24)
CPSQCSCSGTEVHCQRKSLASVPAGIPTTTRVLYLHVNEITKFEPGVFDR
LVNLQQLYLGGNQLSALPDGVFDRLTQLTRLDLYNNQLTVLPAGVFDRLV
NLQTLDLHNNQLKSIPRGAFDNLKSLTHIWLFGNPWDCACSDILYLSGWL
GQHAGKEQGQAVCSGTNTPVRAVTEASTSPSKCP,
which can be encoded by the nucleic acid sequence
(SEQ ID NO: 25)
TGCCCCAGCCAGTGCAGCTGCTCCGGCACAGAAGTGCATTGCCAGAGAAA
GTCCCTGGCCTCTGTGCCTGCCGGCATTCCTACCACAACCAGAGTGCTGT
ACCTGCACGTGAACGAGATCACCAAGTTCGAGCCCGGCGTGTTCGACAGA
CTGGTCAATCTCCAGCAGCTGTACCTCGGCGGCAATCAGCTTTCTGCTCT
GCCCGATGGGGTGTTCGATAGGCTGACCCAGCTGACCAGACTGGACCTGT
ATAACAATCAGCTGACCGTGCTGCCAGCCGGCGTTTTCGATCGGCTCGTG
AATCTCCAGACTCTGGACCTGCACAACAACCAGTTGAAGTCTATCCCCAG
AGGGGCCTTCGACAACCTGAAGTCTCTGACCCACATCTGGCTGTTCGGCA
ACCCCTGGGATTGCGCCTGTAGCGACATCCTGTATCTGTCTGGCTGGCTG
GGACAGCACGCCGGCAAAGAACAAGGACAGGCTGTGTGCAGCGGCACCAA
TACTCCAGTCAGAGCCGTGACCGAGGCCAGCACAAGCCCTTCTAAATGCC
CT.

However, it should be noted that a targeting domain that is capable of binding to CD5 is expected to function. In some embodiments, a chimeric targeting polypeptide and lipid bilayer particle that comprise such polypeptides, CD5 binding alone is sufficient to deliver the contents (e.g., cargo entity) of the lipid bilayer particle into a recipient cell (e.g., lymphocyte).

In some embodiments, a targeting chimeric polypeptide comprises a secretory signal. In some embodiments, a secretory signal has an amino acid sequence that is at least 80%, at least 85%, at least 90%, at least 95%, at least 96%, at least 97%, at least 98%, or at least 99%, or 100% identical to the amino acid sequence METDTLLLWVLLLWVPGSTGD (SEQ ID NO:38). In some embodiments, a secretory signal is encoded by a polynucleotide having the nucleic acid sequence that is at least 80%, at least 85%, at least 90%, at least 95%, at least 96%, at least 97%, at least 98%, or at least 99%, or 100% identical to the amino acid sequence

(SEQ ID NO: 39)
ATGGAAACGGACACCCTGCTGCTGTGGGTGCTGTTGTTGTGGGTGCCAGG
ATCTACAGGCGAC.

In some embodiments, a targeting chimeric polypeptide comprises a FLAG tag (such as a 3×FLAG tag). In some embodiments, a FLAG tag has an amino acid sequence that is at least 80%, at least 85%, at least 90%, at least 95%, at least 96%, at least 97%, at least 98%, or at least 99% identical to the amino acid sequence DYKDHDGDYKDHDIDYKDDDDK (SEQ ID NO:40). In some embodiments, a secretory signal is encoded by a polynucleotide having the nucleic acid sequence that is at least 80%, at least 85%, at least 90%, at least 95%, at least 96%, at least 97%, at least 98%, or at least 99%, or 100% identical to the amino acid sequence

(SEQ ID NO: 41)
GATTACAAGGACCACGATGGCGACTATAAGGATCACGACATCGACTACAA
GGACGATGACGACAAG.

### Fusogen Entity Polypeptides

Without wishing to be bound by any particular theory, it is proposed that fusogen entity polypeptides as described herein mediate cell entry of an engineered lipid bilayer particle displaying such a fusogen entity polypeptide. Certain fusogen entity polypeptides may mediate a cell entry absent a targeting chimeric polypeptide. However, the present disclosure documents that a combination of fusogen entity polypeptides and targeting chimeric polypeptides as described herein can achieve remarkable specificity, efficiency, and/or effectiveness of cargo delivery from engineered lipid bilayer particles that includes them to particular target cells of interest (e.g., that may be human cells and/or immune cells such as T cells, e.g., human T cells). In some embodiments, a fusogen entity polypeptide is characterized by its ability to mediate fusion between lipid bilayers. In some embodiments, a fusogen entity polypeptide mediates transduction of a recipient cell.

In some embodiments, technologies according to the present disclosure comprise (e.g., utilize) a fusogen entity polypeptide. In some embodiments, technologies according to the present disclosure comprise (e.g., utilize) at least one fusogen entity polypeptide (which typically is present in multiple copies on engineered lipid bilayer particles). In some embodiments, technologies according to the present disclosure comprise one or more fusogen entity polypeptides (e.g., each of which may be present in multiple copies on engineered lipid bilayer particles.

In some embodiments, a fusogen entity polypeptide is a naturally occurring (e.g., wild type) polypeptide. In some embodiments, a fusogen entity polypeptide is native to a particular production cell. In some embodiments, a fusogen entity polypeptide is an engineered polypeptide. In some embodiments, a fusogen entity polypeptide (e.g., an engineered fusogen entity polypeptide) is a variant of a wild type polypeptide and/or of a native polypeptide (e.g., comprising one or more amino acid substitutions).

In some embodiments, a fusogen entity polypeptide comprises a secretory signal. In some embodiments, a fusogen entity polypeptide comprises a fusogen moiety. In some embodiments, a fusogen entity polypeptide comprises a transmembrane domain. In some embodiments, a fusogen entity polypeptide comprises an intraparticle portion.

In some embodiments, a fusogen entity polypeptide consists of or comprises a fusogen moiety and a transmembrane portion.

In some embodiments, the order from the N-terminal to the C-terminal of a fusion entity polypeptide is as follows: a secretory signal, a fusogen moiety, a transmembrane portion, a fusogen intraparticle portion, or a combination thereof.

In some embodiments, a fusogen entity polypeptide, or a fusogen moiety thereof, has an amino acid sequence that includes a characteristic sequence element and/or shares an overall degree of sequence identity with a reference fusogen entity polypeptide (e.g., a wild type fusogen entity polypeptide, and/or a fusogen entity polypeptide. In some embodiments, a fusogen entity polypeptide is a variant of such a reference fusogen entity polypeptide. In some embodiments, a fusogen entity polypeptide includes one or more modifications, such as glycosylation, lipidation, phosphorylation, etc.

In some embodiments, a fusogen entity polypeptide is a constitutive fusogen entity polypeptide in that its fusogenic activity does not depend on a particular stimulus or condition.

In some embodiments, a fusogen entity polypeptide is a conditional fusogen entity polypeptide in that its fusogenic activity is dependent upon or triggered by a particular stimulus or condition (e.g., pH, temperature, radiation (e.g., nuclear, ultraviolet, visual, etc), electric signal, magnetic field, etc., or a combination thereof).

In some embodiments, a fusogen entity polypeptide binds to a specific target driving fusion. In some embodiments, a fusogen entity polypeptide binds to low-density lipoprotein (LDL). In some embodiments, a fusogen entity polypeptide binds to a receptor displayed on recipient cell(s).

In some embodiments, a fusogen entity polypeptide comprises a fusogen moiety. Fusogen moieties as provided herein mediate entry of an engineered lipid bilayer particle displaying a fusogen entity polypeptide comprising a fusogen moiety into a recipient cell. In some embodiments, a fusogen moiety mediates transduction of a lipid bilayer particle to a recipient cell. Fusogen moieties cover moieties or functional portions thereof that are characterized in that they promote fusion between lipid bilayers.

In some embodiments, a fusogen moiety is displayed on the surface of an engineered lipid bilayer particle (i.e. arranged so that the fusogen moiety is on the surface of the particle). It may be displayed in a way that the fusogen moiety can interact with a target ligand on the surface of a recipient cell. A fusogen entity may be displayed in a way that promotes fusion of the lipid bilayer of the engineered lipid bilayer particle with the lipid bilayer of a recipient cell.

In some embodiments, a fusogen moiety targets a specific epitope on recipient cells such that binding of this target epitope enables or enhances uptake, fusion and/or functional delivery of the cargo contained within the engineered lipid bilayer particle. Such a target epitope may be expressed on all cells (a universal feature of the cell surface), or on a subset of cells, or on cells that occupy a subset of possible states (e.g., activated T cells versus resting T cells).

In some embodiments a fusogen moiety mediates fusion between an engineered lipid bilayer particle and a target cell in a manner that does not require target epitope binding by the fusogen.

In some embodiments, fusogen moieties enhance fusion between engineered lipid bilayer particles and recipient cells comparable to fusion between engineered lipid bilayer particles and recipient cells where engineered lipid bilayer particles do not display fusogen entity polypeptides comprising a fusogen moiety.

In some embodiments, a fusogen entity polypeptide comprises a viral fusogen moiety. In some embodiments, a fusogen moiety is an enveloped viral fusogen moiety. In some embodiments, a fusogen moiety is a viral glycoprotein.

In some embodiments, lipid bilayer particles comprise a viral glycoprotein to aid in fusion of a lipid bilayer particle with a recipient cell (e.g., a lymphocyte). Viral glycoprotein can be selected from a lentiviral glycoprotein or a glycoprotein selected from vesicular stomatitis glycoprotein (VSV-G), measles virus glycoprotein H, measles virus glycoprotein F, rabies virus glycoprotein (RVG), gibbon ape leukemia virus glycoprotein (GaLV), amphotropic murine leukemia virus glycoprotein (MLV-A), feline endogenous virus (RD 114) glycoprotein, fowl plague virus (FPV) glycoprotein, Ebola virus (EboV) glycoprotein, vesicular stomatitis virus (VSV) glycoprotein, and lymphocytic choriomeningitis virus (LCMV) glycoprotein. In particular, in some embodiments, the glycoprotein may be a measles virus glycoprotein H, measles virus glycoprotein F, or a combination thereof.

In some embodiments, a fusogen entity polypeptide is a polypeptide from vesicular stomatitis virus, Measles virus, Sindbis virus, Tupaia paramyxovirus, Nipah virus, Chandipura virus, Rabies virus, Lymphocytic choriomeningitis virus, Mokola virus, Ross River virus, Ross River virus, Semliki Forest virus, Venezuelan equine encephalitis virus, Ebola virus, Marburg virus, Lassa virus, Avian leukosis virus, Jaagsiekte sheep retrovirus, Moloney Murine leukemia virus, Gibbon ape leukemia virus, Feline endogenous retrovirus (RD 114), Human T-lymphotropic virus 1, Human foamy virus, Maedi-visna virus, SARS-CoV, SARS-CoV-2, Sendai virus, Respiratory syncytia virus, Human parainfluenza virus type 3, Human parainfluenza virus type 4, Hepatitis C virus, Hepatitis C virus, Influenza virus, Fowl plague virus, Autographa californica multiple nucleopolyhedro virus, Baboon endogenous retrovirus, Cocal virus, Japanese encephalitis virus, Dengue virus, Zika virus, West Nile virus, Yellow fever virus, Tick-borne encephalitis virus, Herpes simplex virus 1, Hendra virus, Newcastle disease virus, Epstein Barr virus, Bourbon virus, Varicella-zoster virus, Severe fever with thrombocytopenia virus, Hantavirus, Vaccinia virus, Simian immunodeficiency virus, Human immunodeficiency virus, Junin virus, Machupo virus, Bas-Congo virus, La Crosse virus, Human cytomegalovirus, Human cytomegalovirus, Thogoto virus, or Dhori virus

In some embodiments, a fusogen entity polypeptide is or comprises a glycoprotein selected from vesicular stomatitis glycoprotein (VSV-G).

In some embodiments, a fusogen entity polypeptide is or comprises a wild type VSV-G. In some embodiments, a wild type VSV-G has an amino acid sequence that is at least 80%, at least 85%, at least 90%, at least 95%, at least 96%, at least 97%, at least 98%, or at least identical to amino acid sequence MKCLLYLAFLFIGVNCKFTIVFPHNQKGNWKNVPSNYHYCPSSSDLNWHNDLIGTALQ VKMPKSHKAIQADGWMCHASKWVTTCDFRWYGPKYITHSIRSFTPSVEQCKESIEQTK QGTWLNPGFPPQSCGYATVTDAEAVIVQVTPHHVLVDEYTGEWVDSQFINGKCSNYICP TVHNSTTWHSDYKVKGLCDSNLISMDITFFSEDGELSSLGKEGTGFRSNYFAYETGGKA CKMQYCKHWGVRLPSGVWFEMADKDLFAAARFPECPEGSSISAPSQTSVDVSLIQDVE RILDYSLCQETWSKIRAGLPISPVDLSYLAPKNPGTGPAFTIINGTLKYFETRYIRVDIAAPI L SRMVGMISGTTTERELWDDWAPYEDVEIGPNGVLRTSSGYKFPLYMIGHGMLDSDLH LSSKAQVFEHPHIQDAASQLPDDESLFFGDTGLSKNPIELVEGWFSSWKSSIASFFFIIGLII GLFLVLRVGIHLCIKLKHTKKRQIYTDIEMNRLGK (SEQ ID NO: 26). In some embodiments, a wild type VSV-G fusogen moiety has an amino acid sequence that is identical to the amino acid SEQ ID NO: 26.

In some embodiments, wild type VSV-G is encoded by atgaagtgccttttgtacttagcctttttattcattggggtgaattgcaagttcaccatagtttttccacacaaccaaaaaggaaactggaaaaatg ttccttctaattaccattattgcccgtcaagctcagatttaaattggcataatgacttaataggcacagccttacaagtcaaaatgcccaagagtc acaaggctattcaagcagacggttggatgtgtcatgcttccaaatgggtcactacttgtgatttccgctggtatggaccgaagtatataacacat tccatccgatccttcactccatctgtagaacaatgcaaggaaagcattgaacaaacgaaacaaggaacttggctgaatccaggcttccctcct caaagttgtggatatgcaactgtgacggatgccgaagcagtgattgtccaggtgactcctcaccatgtgctggttgatgaatacacaggaga atgggttgattcacagttcatcaacggaaaatgcagcaattacatatgccccactgtccataactctacaacctggcattctgactataaggtca aagggctatgtgattctaacctcatttccatggacatcaccttcttctcagaggacggagagctatcatccctgggaaaggagggcacaggg ttcagaagtaactactttgcttatgaaactggaggcaaggcctgcaaaatgcaatactgcaagcattggggagtcagactcccatcaggtgtc tggttcgagatggctgataaggatctctttgctgcagccagattccctgaatgcccagaagggtcaagtatctctgctccatctcagacctcag tggatgtaagtctaattcaggacgttgagaggatcttggattattccctctgccaagaaacctggagcaaaatcagagcgggtcttccaatctc tccagtggatctcagctatcttgctcctaaaaacccaggaaccggtcctgctttcaccataatcaatggtaccctaaaatactttgagaccagat acatcagagtcgatattgctgctccaatcctctcaagaatggtcggaatgatcagtggaactaccacagaaagggaactgtgggatgactgg gcaccatatgaagacgtggaaattggacccaatggagttctgaggaccagttcaggatataagtttcctttatacatgattggacatggtatgtt ggactccgatcttcatcttagctcaaaggctcaggtgttcgaacatcctcacattcaagacgctgcttcgcaacttcctgatgatgagagtttatt ttttggtgatactgggctatccaaaaatccaatcgagcttgtagaaggttggttcagtagttggaaaagctctattgcctcttttttctttatcatag ggttaatcattggactattcttggttctccgagttggtatccatctttgcattaaattaaagcacaccaagaaaagacagatttatacagacatag agatgaaccgacttggaaag (SEQ ID NO: 27), or a variant nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 27, or a functional fragment thereof.

In some embodiments, a fusogen entity polypeptide comprises a fusogen moiety. In some embodiments, a fusogen moiety is or comprises a fragment of a wild type VSV-G. In some embodiments, a wild type VSV-G fusogen moiety has an amino acid sequence that is at least 80%, at least 85%, at least 90%, at least 95%, at least 96%, at least 97%, at least 98%, or at least 99% identical to amino acid sequence KFTIVFPHNQKGNWKNVPSNYHYCPSSSDLNWHNDLIGTALQVKMPKSHKAIQADGW MCHASKWVTTCDFRWYGPKYITHSIRSFTPSVEQCKESIEQTKQGTWLNPGFPPQSCGY ATVTDAEAVIVQVTPHHVLVDEYTGEWVDSQFINGKCSNYICPTVHNSTTWHSDYKVK GLCDSNLISMDITFFSEDGELSSLGKEGTGFRSNYFAYETGGKACKMQYCKHWGVRLPS GVWFEMADKDLFAAARFPECPEGSSISAPSQTSVDVSLIQDVERILDYSLCQETWSKIRA GLPISPVDLSYLAPKNPGTGPAFTIINGTLKYFETRYIRVDIAAPILSRMVGMISGTTTERE LWDDWAPYEDVEIGPNGVLRTSSGYKFPLYMIGHGMLDSDLHLSSKAQVFEHPHIQDA ASQLPDDESLFFGDTGLSKNPIELVEGWFSSWKSS (SEQ ID NO: 30). In some embodiments, a wild type VSV-G fusogen moiety has an amino acid sequence that is identical to the amino acid SEQ ID NO: 30.

In some embodiments, a wild type VSV-G fusogen moiety is encoded by atgaagtgccttttgtacttagcctttttattcattggggtgaattgcaagttcaccatagtttttccacacaaccaaaaaggaaactggaaaaatg ttccttctaattaccattattgcccgtcaagctcagatttaaattggcataatgacttaataggcacagccttacaagtcaaaatgcccaagagtc acaaggctattcaagcagacggttggatgtgtcatgcttccaaatgggtcactacttgtgatttccgctggtatggaccgaagtatataacacat tccatccgatccttcactccatctgtagaacaatgcaaggaaagcattgaacaaacgaaacaaggaacttggctgaatccaggcttccctcct caaagttgtggatatgcaactgtgacggatgccgaagcagtgattgtccaggtgactcctcaccatgtgctggttgatgaatacacaggaga atgggttgattcacagttcatcaacggaaaatgcagcaattacatatgccccactgtccataactctacaacctggcattctgactataaggtca aagggctatgtgattctaacctcatttccatggacatcaccttcttctcagaggacggagagctatcatccctgggaaaggagggcacaggg ttcagaagtaactactttgcttatgaaactggaggcaaggcctgcaaaatgcaatactgcaagcattggggagtcagactcccatcaggtgtc tggttcgagatggctgataaggatctctttgctgcagccagattccctgaatgcccagaagggtcaagtatctctgctccatctcagacctcag tggatgtaagtctaattcaggacgttgagaggatcttggattattccctctgccaagaaacctggagcaaaatcagagcgggtcttccaatctc tccagtggatctcagctatcttgctcctaaaaacccaggaaccggtcctgctttcaccataatcaatggtaccctaaaatactttgagaccagat acatcagagtcgatattgctgctccaatcctctcaagaatggtcggaatgatcagtggaactaccacagaaagggaactgtgggatgactgg gcaccatatgaagacgtggaaattggacccaatggagttctgaggaccagttcaggatataagtttcctttatacatgattggacatggtatgtt ggactccgatcttcatcttagctcaaaggctcaggtgttcgaacatcctcacattcaagacgctgcttcgcaacttcctgatgatgagagtttatt ttttggtgatactgggctatccaaaaatccaatcgagcttgtagaaggttggttcagtagttggaaaagctctattgcctcttttttctttatcatag ggttaatcattggactattcttggttctccgagttggtatccatctttgcattaaattaaagcacaccaagaaaagacagatttatacagacatag agatgaaccgacttggaaag (SEQ ID NO: 31), or a variant nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 31, or a functional fragment thereof.

In some embodiments, a fusogen entity polypeptide is or comprises a mutated VSV-G. In some embodiments, a mutated VSV-G has an amino acid sequence that is at least 80%, at least 85%, at least 90%, at least 95%, at least 96%, at least 97%, at least 98%, or at least 99% identical to amino acid sequence MKCLLYLAFLFIGVNCKFTIVFPHNQKGNWKNVPSNYHYCPSSSDLNWHNDLIGTALQ VKMPQSHKAIQADGWMCHASKWVTTCDFRWYGPKYITHSIRSFTPSVEQCKESIEQTK QGTWLNPGFPPQSCGYATVTDAEAVIVQVTPHHVLVDEYTGEWVDSQFINGKCSNYICP TVHNSTTWHSDYKVKGLCDSNLISMDITFFSEDGELSSLGKEGTGFRSNYFAYETGGKA CKMQYCKHWGVRLPSGVWFEMADKDLFAAARFPECPEGSSISAPSQTSVDVSLIQDVE RILDYSLCQETWSKIRAGLPISPVDLSYLAPKNPGTGPAFTIINGTLKYFETRYIRVDIAAPI LSRMVGMISGTTTEAELWDDWAPYEDVEIGPNGVLRTSSGYKFPLYMIGHGMLDSDLH LSSKAQVFEHPHIQDAASQLPDDESLFFGDTGLSKNPIELVEGWFSSWKSSIASFFFIIGLII GLFLVLRVGIHLCIKLKHTKKRQIYTDIEMNRLGK (SEQ ID NO: 28). In some embodiments, a mutated VSV-G fusogen entity polypeptide has an amino acid sequence that is identical to the amino acid SEQ ID NO: 28.

In some embodiments, a mutated VSV-G is encoded by atgaagtgccttttgtacttagcctttttattcattggggtgaattgcaagttcaccatagtttttccacacaaccaaaaaggaaactggaaaaatg ttccttctaattaccattattgcccgtcaagctcagatttaaattggcataatgacttaataggcacagccttacaagtcaaaatgccccagagtc acaaggctattcaagcagacggttggatgtgtcatgcttccaaatgggtcactacttgtgatttccgctggtatggaccgaagtatataacacat tccatccgatccttcactccatctgtagaacaatgcaaggaaagcattgaacaaacgaaacaaggaacttggctgaatccaggcttccctcct caaagttgtggatatgcaactgtgacggatgccgaagcagtgattgtccaggtgactcctcaccatgtgctggttgatgaatacacaggaga atgggttgattcacagttcatcaacggaaaatgcagcaattacatatgccccactgtccataactctacaacctggcattctgactataaggtca aagggctatgtgattctaacctcatttccatggacatcaccttcttctcagaggacggagagctatcatccctgggaaaggagggcacaggg ttcagaagtaactactttgcttatgaaactggaggcaaggcctgcaaaatgcaatactgcaagcattggggagtcagactcccatcaggtgtc tggttcgagatggctgataaggatctctttgctgcagccagattccctgaatgcccagaagggtcaagtatctctgctccatctcagacctcag tggatgtaagtctaattcaggacgttgagaggatcttggattattccctctgccaagaaacctggagcaaaatcagagcgggtcttccaatctc tccagtggatctcagctatcttgctcctaaaaacccaggaaccggtcctgctttcaccataatcaatggtaccctaaaatactttgagaccagat acatcagagtcgatattgctgctccaatcctctcaagaatggtcggaatgatcagtggaactaccacagaagcggaactgtgggatgactgg gcaccatatgaagacgtggaaattggacccaatggagttctgaggaccagttcaggatataagtttcctttatacatgattggacatggtatgtt ggactccgatcttcatcttagctcaaaggctcaggtgttcgaacatcctcacattcaagacgctgcttcgcaacttcctgatgatgagagtttatt ttttggtgatactgggctatccaaaaatccaatcgagcttgtagaaggttggttcagtagttggaaaagctctattgcctcttttttctttatcatag ggttaatcattggactattcttggttctccgagttggtatccatctttgcattaaattaaagcacaccaagaaaagacagatttatacagacatag agatgaaccgacttggaaag (SEQ ID NO: 29), or a variant nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 29, or a functional fragment thereof.

In some embodiments, a fusogen entity polypeptide comprises a fusogen moiety. In some embodiments, a fusogen moiety is or comprises a fragment of a mutated VSV-G. In some embodiments, a mutated VSV-G fusogen moiety has an amino acid sequence that is at least 80%, at least 85%, at least 90%, at least 95%, at least 96%, at least 97%, at least 98%, or at least 99% identical to amino acid sequence KFTIVFPHNQKGNWKNVPSNYHYCPSSSDLNWHNDLIGTALQVKMPQSSHKAIQADGW MCHASKWVTTCDFRWYGPKYITHSIRSFTPSVEQCKESIEQTKQGTWLNPGFPPQSCGY ATVTDAEAVIVQVTPHHVLVDEYTGEWVDSQFINGKCSNYICPTVHNSTTWHSDYKVK GLCDSNLISMDITFFSEDGEL SSLGKEGTGFRSNYFAYETGGKACKMQYCKHWGVRLPS GVWFEMADKDLFAAARFPECPEGSSISAPSQTSVDVSLIQDVERILDYSLCQETWSKIRA GLPISPVDLSYLAPKNPGTGPAF TIINGTLKYFETRYIRVDIAAPILSRMVGMISGTT TEAE LWDDWAPYEDVEIGPNGVLRTSSGYKFPLYMIGHGMLDSDLHLSSKAQVFEHPHIQDA ASQLPDDESLFFGDTGLSKNPIELVEGWFSSWKSS (SEQ ID NO: 32). In some embodiments, a mutated VSV-G fusogen moiety has an amino acid sequence that is identical to the amino acid SEQ ID NO: 32.

In some embodiments, a mutated VSV-G fusogen moiety is encoded by atgaagtgccttttgtacttagcctttttattcattggggtgaattgcaagttcaccatagtttttccacacaaccaaaaaggaaactggaaaaatg ttccttctaattaccattattgcccgtcaagctcagatttaaattggcataatgacttaataggcacagccttacaagtcaaaatgccccagagtc acaaggctattcaagcagacggttggatgtgtcatgcttccaaatgggtcactacttgtgatttccgctggtatggaccgaagtatataacacat tccatccgatccttcactccatctgtagaacaatgcaaggaaagcattgaacaaacgaaacaaggaacttggctgaatccaggcttccctcct caaagttgtggatatgcaactgtgacggatgccgaagcagtgattgtccaggtgactcctcaccatgtgctggttgatgaatacacaggaga atgggttgattcacagttcatcaacggaaaatgcagcaattacatatgccccactgtccataactctacaacctggcattctgactataaggtca aagggctatgtgattctaacctcatttccatggacatcaccttcttctcagaggacggagagctatcatccctgggaaaggagggcacaggg ttcagaagtaactactttgcttatgaaactggaggcaaggcctgcaaaatgcaatactgcaagcattggggagtcagactcccatcaggtgtc tggttcgagatggctgataaggatctctttgctgcagccagattccctgaatgcccagaagggtcaagtatctctgctccatctcagacctcag tggatgtaagtctaattcaggacgttgagaggatcttggattattccctctgccaagaaacctggagcaaaatcagagcgggtcttccaatctc tccagtggatctcagctatcttgctcctaaaaacccaggaaccggtcctgctttcaccataatcaatggtaccctaaaatactttgagaccagat acatcagagtcgatattgctgctccaatcctctcaagaatggtcggaatgatcagtggaactaccacagaagcggaactgtgggatgactgg gcaccatatgaagacgtggaaattggacccaatggagttctgaggaccagttcaggatataagtttcctttatacatgattggacatggtatgtt ggactccgatcttcatcttagctcaaaggctcaggtgttcgaacatcctcacattcaagacgctgcttcgcaacttcctgatgatgagagtttatt ttttggtgatactgggctatccaaaaatccaatcgagcttgtagaaggttggttcagtagttggaaaagctct (SEQ ID NO: 33), or a variant nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 33, or a functional fragment thereof.

In some embodiments, fusogen entity polypeptide comprises an intraparticle portion. In some embodiments, an intraparticle polypeptide portion has an amino acid sequence that is at least 80%, at least 85%, at least 90%, at least 95%, at least 96%, at least 97%, at least 98%, or at least 99% identical to the amino acid sequence KLKHTKKRQIYTDIEMNRLGK (SEQ ID NO:36). In some embodiments, an intraparticle polypeptide portion is encoded by a polynucleotide having the nucleic acid sequence that is at least 80%, at least 85%, at least 90%, at least 95%, at least 96%, at least 97%, at least 98%, or at least 99% identical to the amino acid sequence aaattaaagcacaccaagaaaagacagatttatacagacatagagatgaaccgacttggaaag (SEQ ID NO: 37).

In some embodiments, a fusogen entity polypeptide is or comprises a measles virus polypeptide, such as a measles virus glycoprotein. In some embodiments, a fusogen entity polypeptide is or comprises a measles virus glycoprotein H and/or F.

In some embodiments, a fusogen entity polypeptide is or comprises a sindbis virus polypeptide, such as a sindbis virus glycoprotein. In some embodiments, a fusogen entity polypeptide is or comprises a sindbis virus glycoprotein E1 and/or E2.

In some embodiments, a fusogen entity polypeptide is or comprises a tupaia paramyxovirus polypeptide, such as a tupaia paramyxovirus glycoprotein. In some embodiments, a fusogen entity polypeptide is or comprises a tupaia paramyxovirus glycoprotein H and/or F.

In some embodiments, a fusogen entity polypeptide is or comprises a nipah virus polypeptide, such as a nipah virus glycoprotein. In some embodiments, a fusogen entity polypeptide is or comprises a nipah virus glycoprotein G and/or F.

In some embodiments, a fusogen entity polypeptide is or comprises a chandipura virus polypeptide, such as a chandipura virus glycoprotein. In some embodiments, a fusogen entity polypeptide is or comprises a chandipura virus glycoprotein G.

In some embodiments, a fusogen entity polypeptide is or comprises a rabies virus polypeptide, such as a rabies virus glycoprotein. In some embodiments, a fusogen entity polypeptide is or comprises a rabies virus glycoprotein G.

In some embodiments, a fusogen entity polypeptide is or comprises a lymphocytic choriomeningitis virus polypeptide, such as a lymphocytic choriomeningitis virus glycoprotein. In some embodiments, a fusogen entity polypeptide is or comprises a lymphocytic choriomeningitis virus glycoprotein GP-1 and/or GP-2.

In some embodiments, a fusogen entity polypeptide is or comprises a mokola virus polypeptide, such as a mokola virus glycoprotein. In some embodiments, a fusogen entity polypeptide is or comprises a mokola virus glycoprotein G.

In some embodiments, a fusogen entity polypeptide is or comprises a ross river virus polypeptide, such as a ross river virus glycoprotein. In some embodiments, a fusogen entity polypeptide is or comprises a ross river virus glycoprotein E1 and/or E2.

In some embodiments, a fusogen entity polypeptide is or comprises a semliki forest virus polypeptide, such as a semliki forest virus glycoprotein. In some embodiments, a fusogen entity polypeptide is or comprises a semliki forest virus glycoprotein E1 and/or E2.

In some embodiments, a fusogen entity polypeptide is or comprises a venezuelan equine encephalitis virus polypeptide, such as a venezuelan equine encephalitis virus glycoprotein. In some embodiments, a fusogen entity polypeptide is or comprises a venezuelan equine encephalitis virus glycoprotein E1 and/or E2.

In some embodiments, a fusogen entity polypeptide is or comprises an ebola virus polypeptide, such as an ebola virus glycoprotein. In some embodiments, a fusogen entity polypeptide is or comprises an ebola virus glycoprotein GP.

In some embodiments, a fusogen entity polypeptide is or comprises a marburg virus polypeptide, such as a marburg virus glycoprotein. In some embodiments, a fusogen entity polypeptide is or comprises a marburg virus glycoprotein GP.

In some embodiments, a fusogen entity polypeptide is or comprises a lassa virus polypeptide, such as a lassa virus glycoprotein. In some embodiments, a fusogen entity polypeptide is or comprises a lassa virus glycoprotein GPC.

In some embodiments, a fusogen entity polypeptide is or comprises an avian leucosis virus polypeptide, such as an avian leucosis virus glycoprotein. In some embodiments, a fusogen entity polypeptide is or comprises an avian leucosis virus envelope glycoprotein.

In some embodiments, a fusogen entity polypeptide is or comprises a jaagsiekte sheep virus polypeptide, such as a jaagsiekte sheep virus glycoprotein. In some embodiments, a fusogen entity polypeptide is or comprises a jaagsiekte sheep virus envelope glycoprotein.

In some embodiments, a fusogen entity polypeptide is or comprises a moloney murine leukemia virus polypeptide, such as a moloney murine leukemia virus glycoprotein. In some embodiments, a fusogen entity polypeptide is or comprises a moloney murine leukemia virus envelope glycoprotein.

In some embodiments, a fusogen entity polypeptide is or comprises a gibbon ape leukemia virus polypeptide, such as a gibbon ape leukemia virus glycoprotein. In some embodiments, a fusogen entity polypeptide is or comprises a gibbon ape leukemia virus envelope glycoprotein.

In some embodiments, a fusogen entity polypeptide is or comprises a feline endogenous retrovirus polypeptide, such as a feline endogenous retrovirus glycoprotein. In some embodiments, a fusogen entity polypeptide is or comprises a RD 114 glycoprotein.

In some embodiments, a fusogen entity polypeptide is or comprises a human T-lymphocyte virus 1 polypeptide, such as a human T-lymphocyte virus 1 glycoprotein. In some embodiments, a fusogen entity polypeptide is or comprises a human T-lymphocyte virus 1 glycoprotein SU and/or TM. In some embodiments, a fusogen entity polypeptide is or comprises a human T-lymphocyte virus 1 glycoprotein gp46.

In some embodiments, a fusogen entity polypeptide is or comprises a human foamy virus polypeptide, such as a human foamy virus glycoprotein. In some embodiments, a fusogen entity polypeptide is or comprises a human foamy virus envelope glycoprotein.

In some embodiments, a fusogen entity polypeptide is or comprises a maedi-visna virus polypeptide, such as a maedi-visna virus glycoprotein. In some embodiments, a fusogen entity polypeptide is or comprises a maedi-visna virus envelope glycoprotein.

In some embodiments, a fusogen entity polypeptide is or comprises a SARS-CoV polypeptide (e.g., SARS-CoV 2), such as a SARS-CoV glycoprotein. In some embodiments, a fusogen entity polypeptide is or comprises a SARS-CoV spike glycoprotein (S).

In some embodiments, a fusogen entity polypeptide is or comprises a sendai virus polypeptide, such as a sendai virus glycoprotein. In some embodiments, a fusogen entity polypeptide is or comprises a sendai virus glycoprotein HN and/or F.

In some embodiments, a fusogen entity polypeptide is or comprises a respiratory syncytia virus polypeptide, such as a respiratory syncytia virus glycoprotein. In some embodiments, a fusogen entity polypeptide is or comprises a respiratory syncytia virus glycoprotein G and/or F.

In some embodiments, a fusogen entity polypeptide is or comprises a human parainfluenza virus type 3 and/or type 4 polypeptide, such as a respiratory human parainfluenza virus type 3 and/or type 4 glycoprotein. In some embodiments, a fusogen entity polypeptide is or comprises a human parainfluenza virus type 3 and/or type 4 glycoprotein HN and/or F.

In some embodiments, a fusogen entity polypeptide is or comprises a hepatitis C virus polypeptide, such as a hepatitis C virus glycoprotein. In some embodiments, a fusogen entity polypeptide is or comprises a hepatitis C virus glycoprotein E1 and/or E2.

In some embodiments, a fusogen entity polypeptide is or comprises an influenza virus polypeptide, such as an influenza virus glycoprotein. In some embodiments, a fusogen entity polypeptide is or comprises an influenza virus glycoprotein HA and/or NA.

In some embodiments, a fusogen entity polypeptide is or comprises a fowl plague virus polypeptide, such as a fowl plague virus glycoprotein. In some embodiments, a fusogen entity polypeptide is or comprises a fowl plague virus glycoprotein HA.

In some embodiments, a fusogen entity polypeptide is or comprises an Autographa californica multiple nucleopolyhedro virus polypeptide, such an Autographa californica multiple nucleopolyhedro virus glycoprotein. In some embodiments, a fusogen entity polypeptide is or comprises an Autographa californica multiple nucleopolyhedro virus glycoprotein gp64.

In some embodiments, a fusogen entity polypeptide is or comprises a baboon endogenous retrovirus polypeptide, such a baboon endogenous retrovirus glycoprotein. In some embodiments, a fusogen entity polypeptide is or comprises a baboon endogenous retrovirus glycoprotein envelope.

In some embodiments, a fusogen entity polypeptide is or comprises a cocal virus polypeptide, such a cocal virus glycoprotein (G).

In some embodiments, a fusogen entity polypeptide is or comprises a Japanese encephalitis virus polypeptide, such a Japanese encephalitis virus glycoprotein. In some embodiments, a fusogen entity polypeptide is or comprises a Japanese encephalitis virus glycoprotein E.

In some embodiments, a fusogen entity polypeptide is or comprises a dengue virus polypeptide, such a dengue virus glycoprotein. In some embodiments, a fusogen entity polypeptide is or comprises a dengue virus glycoprotein E.

In some embodiments, a fusogen entity polypeptide is or comprises a zika virus polypeptide, such a zika virus glycoprotein. In some embodiments, a fusogen entity polypeptide is or comprises a zika virus glycoprotein E.

In some embodiments, a fusogen entity polypeptide is or comprises a west nile virus polypeptide, such a west nile virus glycoprotein. In some embodiments, a fusogen entity polypeptide is or comprises a west nile virus glycoprotein E.

In some embodiments, a fusogen entity polypeptide is or comprises a yellow fever virus polypeptide, such a yellow fever virus glycoprotein. In some embodiments, a fusogen entity polypeptide is or comprises a yellow fever virus glycoprotein E.

In some embodiments, a fusogen entity polypeptide is or comprises a tick-borne encephalitis virus polypeptide, such a tick-borne encephalitis virus glycoprotein. In some embodiments, a fusogen entity polypeptide is or comprises a tick-borne encephalitis virus glycoprotein E.

In some embodiments, a fusogen entity polypeptide is or comprises a herpes simplex virus polypeptide, such a herpes simplex virus glycoprotein. In some embodiments, a fusogen entity polypeptide is or comprises a herpes simplex virus glycoprotein HSV-1 gB, HSV-1 gH, HSV-1 gL, and/or HSV-1 gD.

In some embodiments, a fusogen entity polypeptide is or comprises a hendra virus polypeptide, such a hendra virus glycoprotein. In some embodiments, a fusogen entity polypeptide is or comprises a hendra virus glycoprotein G and/or F.

In some embodiments, a fusogen entity polypeptide is or comprises a Newcastle disease virus polypeptide, such a Newcastle disease virus glycoprotein. In some embodiments, a fusogen entity polypeptide is or comprises a Newcastle disease virus glycoprotein F1 and/or F2.

In some embodiments, a fusogen entity polypeptide is or comprises an Epstein Barr virus polypeptide, such an Epstein Barr virus glycoprotein. In some embodiments, a fusogen entity polypeptide is or comprises an Epstein Barr virus glycoprotein gB, gH and/or gL. In some embodiments, a fusogen entity polypeptide is or comprises an Epstein Barr virus glycoprotein gp42.

In some embodiments, a fusogen entity polypeptide is or comprises a bourbon virus polypeptide, such a bourbon virus glycoprotein. In some embodiments, a fusogen entity polypeptide is or comprises a bourbon virus glycoprotein Gp.

In some embodiments, a fusogen entity polypeptide is or comprises a varicella-zoster virus polypeptide, such a varicella-zoster virus glycoprotein. In some embodiments, a fusogen entity polypeptide is or comprises a varicella-zoster virus glycoprotein gB, gH, gE, and/or gL.

In some embodiments, a fusogen entity polypeptide is or comprises a severe fever with thrombocytopenia virus polypeptide, such a severe fever with thrombocytopenia virus glycoprotein. In some embodiments, a fusogen entity polypeptide is or comprises a severe fever with thrombocytopenia virus glycoprotein gB, gH, and/or gL.

In some embodiments, a fusogen entity polypeptide is or comprises a hantavirus polypeptide, such a hantavirus glycoprotein. In some embodiments, a fusogen entity polypeptide is or comprises a hantavirus glycoprotein Gn and/or Gc.

In some embodiments, a fusogen entity polypeptide is or comprises a vaccinia virus polypeptide, such a vaccinia virus glycoprotein. In some embodiments, a fusogen entity polypeptide is or comprises a vaccinia virus glycoprotein A28, A21, A16, F9, G9, G3, H2, J5, L5, L1, A33, A34, B5, and/or O3.

In some embodiments, a fusogen entity polypeptide is or comprises a simian immunedeficient virus polypeptide, such a simian immunedeficient virus glycoprotein. In some embodiments, a fusogen entity polypeptide is or comprises a simian immunedeficient virus Env glycoprotein.

In some embodiments, a fusogen entity polypeptide is or comprises a human immunedeficient virus (e.g., HIV-1) polypeptide, such a human immunedeficient virus glycoprotein. In some embodiments, a fusogen entity polypeptide is or comprises a human immunedeficient virus Env glycoprotein.

In some embodiments, a fusogen entity polypeptide is or comprises a junin virus polypeptide, such a junin virus glycoprotein. In some embodiments, a fusogen entity polypeptide is or comprises a junin virus glycoprotein complex GPC.

In some embodiments, a fusogen entity polypeptide is or comprises a machupo virus polypeptide, such a machupo virus glycoprotein. In some embodiments, a fusogen entity polypeptide is or comprises a machupo virus glycoprotein complex GPC.

In some embodiments, a fusogen entity polypeptide is or comprises a bas-congo virus polypeptide, such a bas-congo virus glycoprotein. In some embodiments, a fusogen entity polypeptide is or comprises a bas-congo virus glycoprotein G.

In some embodiments, a fusogen entity polypeptide is or comprises a la crosse virus polypeptide, such a la crosse virus glycoprotein. In some embodiments, a fusogen entity polypeptide is or comprises a la crosse virus glycoprotein Gc and/or Gn.

In some embodiments, a fusogen entity polypeptide is or comprises a human cytomegalovirus polypeptide, such a human cytomegalovirus glycoprotein. In some embodiments, a fusogen entity polypeptide is or comprises a human cytomegalovirus glycoprotein gH, gL, gO, UL128, UL130, and/or UL131A.

In some embodiments, a fusogen entity polypeptide is or comprises a thogoto virus polypeptide, such a thogoto virus glycoprotein. In some embodiments, a fusogen entity polypeptide is or comprises a thogoto virus glycoprotein Gp.

In some embodiments, a fusogen entity polypeptide is or comprises a dhori virus polypeptide, such a dhori virus glycoprotein. In some embodiments, a fusogen entity polypeptide is or comprises a dhori virus glycoprotein Gp.

In some embodiments, a fusogen entity polypeptide comprises a non-viral fusogen moiety.

In some embodiments, a fusogen entity polypeptide comprises a human fusogen moiety.

In some embodiments, a fusogen entity polypeptide is or comprises a human fusogen moiety selected from the group consisting of Syncytin-1, Syncytin-2, CD9, CD81, myomarker, CD200 (OX-2G), DC-STAMP, OC-STAMP, E-Cadherin (CADH1), Cadherin 11 (CAD 11), matrix meralloproteinase-9, zonula occludens-1 (ZO-1), myomerger, Annexin A1, Annexin A5, CD44, P2X purinoceptor 7, IZUMO1, Juno, StartD7, receptor-like 1, associated protein 4, CD63, connexin 43 (Cx43), CD36, MFR, tumor-associated member 1, GLPR1-like protein 1, associated protein 43, ERVV-1, ERVV-2, ERVH48-1, ERVMER34-1, ERV3-1, and ERVK13-1.

In some embodiments, a fusogen entity polypeptide is or comprises a human fusogen moiety selected from the group consisting of Syncytin-1, Syncytin-2, CD9, CD81, myomarker, CD200 (OX-2G), DC-STAMP, OC-STAMP, E-Cadherin (CADH1), Cadherin 11 (CAD 11), matrix meralloproteinase-9, zonula occludens-1 (ZO-1), myomerger, Annexin A1, Annexin A5, CD44, P2X purinoceptor 7, IZUMO1, Juno, StartD7, receptor-like 1, associated protein 4, CD63, connexin 43 (Cx43), CD36, MFR, tumor-associated member 1, GLPR1-like protein 1, and associated protein 43.

### Transmembrane Domains

In some embodiments, polypeptides describes herein (e.g., targeting chimeric polypeptides, fusogen entity polypeptides, cargo loading polypeptides, etc.) comprise a transmembrane domain (TMD). In some embodiments, a transmembrane domain allows that a targeting domain is displayed on the surface of a lipid bilayer particle. In some embodiments, a membrane association portion positions an affinity moiety on the surface of a lipid bilayer particle such that it can bind to a recipient cell surface epitope. As used herein the term “transmembrane domain” covers any “membrane association portion” that is capable of association with a membrane (e.g., a lipid bilayer membrane). Those skilled in the art would understand that, in some embodiments, a “transmembrane domain” is a stretch of amino acids that together cause association with a membrane. In some embodiments, a transmembrane domain spans a membrane. In some embodiments, a transmembrane domain does not span a membrane. In some embodiments, a transmembrane domain is associated with a membrane (e.g., a lipid bilayer membrane). In some embodiments, a transmembrane domain is positioned at the C-terminus of a polypeptide. In some embodiments, a transmembrane domain is characterized by a length of about 10 amino acids to about 300 amino acids. In some embodiments, a transmembrane domain is a heterologous transmembrane domain (e.g., relative to another portion of polypeptide).

In some embodiments, a transmembrane domain is a viral transmembrane domain. In some embodiments, a transmembrane domain is a viral envelope transmembrane domain. In some embodiments, a transmembrane domain is a non-viral transmembrane domain. In some embodiments, a transmembrane domain is an engineered transmembrane domain.

Transmembrane domains are known in the art. Transmembrane domains (TMDs) consist predominantly of nonpolar amino acid residues and may traverse the bilayer once (single pass) or several times. TMDs usually consist of a helices. The peptide bond is polar and can include internal hydrogen bonds formed between carbonyl oxygen atoms and amide nitrogen atoms which may be hydrated. Within the lipid bilayer, where water is essentially excluded, peptides usually adopt the α-helical configuration in order to maximize their internal hydrogen bonding. A length of helix of 18-21 amino acid residues is usually sufficient to span the usual width of a lipid bilayer. TMDs that are oriented with an extracytoplasmic N-terminus and a cytoplasmic C-terminus are classified as type I TMDs, and TMDs that are oriented with an extracytoplasmic C-terminus and a cytoplasmic N-terminus are classified as type II TMDs. In some embodiments of the disclosed extracytoplasmic, they are classified as type I or, if cytoplasmic, type II. In some embodiments, a transmembrane domain is a single pass, type I transmembrane domain comprising 18-21 amino acids, where at least about 90% of the amino acids are nonpolar. Suitable TMDs for the disclosed fusion proteins may include, but are not limited to, the transmembrane domain of cellular receptors, such as the platelet-derived growth factor receptor (PDGFR) transmembrane domain. In some embodiments, a transmembrane is a transmembrane domain of Vesicular stomatitis virus, Measles virus, Sindbis virus, Tupaia paramyxovirus, Nipah virus, Chandipura virus, Rabies virus, Lymphocytic choriomeningitis virus, Mokola virus, Ross River virus, Ross River virus, Semliki Forest virus, Venezuelan equine encephalitis virus, Ebola virus, Marburg virus, Lassa virus, Avian leukosis virus, Jaagsiekte sheep retrovirus, Moloney Murine leukemia virus, Gibbon ape leukemia virus, Feline endogenous retrovirus (RD 114), Human T-lymphotropic virus 1, Human foamy virus, Maedi-visna virus, SARS-CoV, SARS-CoV-2, Sendai virus, Respiratory syncytia virus, Human parainfluenza virus type 3, Human parainfluenza virus type 4, Hepatitis C virus, Hepatitis C virus, Influenza virus, Fowl plague virus, Autographa californica multiple nucleopolyhedro virus, Baboon endogenous retrovirus, Cocal virus, Japanese encephalitis virus, Dengue virus, Zika virus, West Nile virus, Yellow fever virus, Tick-borne encephalitis virus, Herpes simplex virus 1, Hendra virus, Newcastle disease virus, Epstein Barr virus, Bourbon virus, Varicella-zoster virus, Severe fever with thrombocytopenia virus, Hantavirus, Vaccinia virus, Simian immunodeficiency virus, Human immunodeficiency virus, Junin virus, Machupo virus, Bas-Congo virus, La Crosse virus, Human cytomegalovirus, Human cytomegalovirus, Thogoto virus, or Dhori virus.

In some embodiments, a PDGFR transmembrane domain comprises an amino acid sequence of SEQ ID NO: 18 (AVGQDTQEVIVVPHSLPFKVVVISAILALVVLTIISLIILIMLWQKKPR), which can be encoded by the nucleotide sequence GCCGTCGGCCAGGACACCCAAGAAGTGATCGTCGTCCCTCACAGCCTGCCTTTCAAG GTGGTGGTCATCAGCGCCATTCTGGCCCTGGTGGTGCTGACCATCATCAGCCTGATC ATCCTGATTATGCTGTGGCAGAAGAAGCCCAGA (SEQ ID NO: 19).

The TMD may be linked directly to the targeting domain (e.g., a scFv) or the TMD may be linked via a linker. In some embodiments, the linker linking the TMD and the targeting domain comprises amino acids sequence of (GGGGS)ₙ(SEQ ID NO: 42), wherein n is 1, 2, 3, 4, 5, 6, 7, 8, 9, 10, 11, 12, 13, 14, 15, or more. In some embodiments, the linker linking the TMD and the targeting peptide comprises: (1) an amino acid sequence selected from SEQ ID NO: 10, SEQ ID NO: 12, SEQ ID NO: 14, SEQ ID NO: 15, SEQ ID NO: 16, and SEQ ID NO: 17; or (2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 10, 12, 14, 15, 16, or 17.

Thus, in some embodiments, the transmembrane domain may comprise AVGQDTQEVIVVPHSLPFKVVVISAILALVVLTIISLIILIMLWQKKPR (SEQ ID NO: 18), a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 18, or a functional fragment thereof.

In some embodiments, a fusogen entity polypeptide comprises a transmembrane domain. In some embodiments, a transmembrane domain is or comprises a wild type VSV-G transmembrane portion or a fragment thereof. In some embodiments, a fusogen entity polypeptide transmembrane domain may comprise IASFFFIIGLIIGLFLVLRVGIHLCI (SEQ ID NO: 22), which can be encoded by the nucleotide sequence attgcctcttttttctttatcatagggttaatcattggactattcttggttctccgagttggtatccatctttgcatt (SEQ ID NO: 23) In some embodiments, a wild type VSV-G fusogen transmembrane portion has an amino acid sequence that is at least 80%, at least 85%, at least 90%, at least 95%, at least 96%, at least 97%, at least 98%, or at least 99% identical to the amino acid SEQ ID NO: 22. In some embodiments, a wild type VSV-G fusogen transmembrane portion has an amino acid sequence that is identical to the amino acid SEQ ID NO: 22.

In some embodiments, a transmembrane domain of a non-human transmembrane domain. In some embodiments, a transmembrane domain of a human transmembrane domain.

In some embodiments, a transmembrane domain integrates into the membrane of a lipid bilayer particle with a high copy number.

### Secretory Signal

In some embodiments, a polypeptide disclosed herein (e.g., a targeting chimeric polypeptide and/or a fusogen entity polypeptide) comprises a secretory signal, e.g., that is functional in mammalian cells. In some embodiments, a utilized secretory signal is a heterologous secretory signal. In some embodiments, a heterologous secretory signal comprises or consists of a non-human secretory signal. In some embodiments, a heterologous secretory signal comprises or consists of a viral secretory signal. In some embodiments, a secretory signal is characterized by a length of about 10 to about 40 amino acids, such as about 20 to about 30 amino acids. In some embodiments, a secretory signal is positioned at the N-terminus of a fusogen entity polypeptide described herein. In some embodiments, a secretory signal preferably allows transport of a fusogen entity polypeptide with which it is associated into a defined cellular compartment, preferably a cell surface, endoplasmic reticulum (ER), Golgi apparatus, or endosomal-lysosomal compartment.

In some embodiments, a secretory signal has an amino acid sequence that is at least 80%, at least 85%, at least 90%, at least 95%, at least 96%, at least 97%, at least 98%, or at least 99% identical to the amino acid sequence MKCLLYLAFLFIGVNC (SEQ ID NO:34). In some embodiments, a secretory signal is encoded by a polynucleotide having the nucleic acid sequence that is at least 80%, at least 85%, at least 90%, at least 95%, at least 96%, at least 97%, at least 98%, or at least 99%, or 100% identical to the amino acid sequence atgaagtgccttttgtacttagcctttttattcattggggtgaattgc (SEQ ID NO: 35).

### Affinity Flags

In some embodiments, a polypeptide disclosed herein (e.g., a targeting chimeric polypeptide and/or a fusogen entity polypeptide) comprises an FLAG tag. In some embodiments, a utilized FLAG tag is a heterologous secretory tag. In some embodiments, a heterologous FLAG tag comprises or consists of a non-human FLAG tag. In some embodiments, a heterologous FLAG tag comprises or consists of a viral FLAG tag. In some embodiments, a FLAG tag signal is characterized by a length of about 15 to 30 amino acids. In some embodiments, an FLAG tag is positioned between a secretory signal sequence and a targeting domain sequence within a targeting chimeric polypeptide described herein. In some embodiments, a polypeptide described herein does not comprise a FLAG tag or any other tag.

### Cargo Entity

In some embodiments, disclosed technologies comprise a cargo entity. In some embodiments, a lipid bilayer particle or a population of lipid bilayer particles comprises a cargo entity. In some embodiments, disclosed technologies comprise one or more cargo entities, such as multiple cargo entities (e.g., a first cargo entity, a second cargo entity, etc. or a combination thereof). Cargo entities described herein can of any chemical class, e.g., polypeptides, nucleic acids, saccharides, lipids, small entities, and combinations thereof.

In some embodiments, a cargo entity is a part of a targeting chimeric polypeptide, fusogen entity polypeptide, or both. In some embodiments, a cargo entity binds to a targeting chimeric polypeptide, a fusogen entity polypeptide, or both.

In some embodiments, a cargo entity is a part of a distinct polypeptide from each of the targeting chimeric polypeptide and the fusogen entity polypeptide. In some embodiments, a cargo entity is linked to a cargo-loading domain. In some embodiments, a cargo-loading domain comprises an abscisic acid-insensitive 1 (ABI1) sequence.

In some embodiments, a cargo-loading domain is linked to a cargo entity and co-expressed with an ABA-binding sequence (e.g., comprising a pyrabactin resistance 1-like (PYL1) sequence), and an abscisic acid (ABA).

In some embodiments, a cargo-loading domain is linked to a cargo entity.

In some embodiments, a cargo-loading domain is linked to a cargo entity and co-expressed with an ABA-binding sequence (e.g., comprising a pyrabactin resistance 1-like (PYL1) sequence).

In some embodiments, a cargo entity is a cytosolic cargo entity or a membrane bound cargo entity.

In some embodiments, a cargo-loading domain comprising an ABI1 sequence may be fused to a membrane polypeptide (i.e., a membrane-bound polypeptide). In other words, in some embodiments, the cargo entity may be a membrane polypeptide. Similarly, in some embodiments the cargo-loading domain comprising an abscisic acid-insensitive 1 (ABI1) sequence may be fused to a cytosloic polypeptide (i.e., a polypeptide that is not membrane-bound). In other words, in some embodiments, the cargo entity may be a cytosolic polypeptide.

The loading system may be used to deliver a single cargo (or single type of cargo) to a desired recipient cell, or it may be used to deliver multiple cargos (e.g., different polypeptides) to a desired cell. For the purposes of embodiments that include multiple different cargos, the disclosed loading system can be used to control the ratios of the various cargos loaded into the lipid bilayer particle and, subsequently, delivered to a desired recipient cell.

In some embodiments, a cargo entity is a polypeptide cargo entity. In some embodiments, a cargo entity is a cytosolic cargo molecule, wherein the cytosolic cargo entity may a peptide, polypeptide, or protein of interest to be delivered to a recipient cell, such as an enzyme, a therapeutic agent (e.g., an antibody, inhibitor, an agonist, and an antagonist), or a fluorescent protein. In some embodiments, a cytosolic polypeptide cargo entity can be selected from any one or more of base editors, prime editors, TALENs, ZFNs, kinases, kinase inhibitors, activators or inhibitors of receptor-signaling, intrabodies, chromatin-modifying synthetic transcription factors, natural transcription factors, and mutant forms thereof. In some embodiments, a cytosolic polypeptide cargo entity is a CRISPR enzyme, e.g., a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is a Cas9 protein (e.g., a naturally-occurring bacterial Cas9 as well as any chimeras, mutants, homologs or orthologs). Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof.

In some embodiments, a cargo entity is a nucleic acid cargo entity. In some embodiments, a nucleic acid cargo entity is a synthetic nucleic acid cargo entity. In some embodiments, a nucleic acid cargo entity comprises chemically modifies nucleotides. Without wishing to be bound by a particular theory, a synthetic nucleic acid cargo entity and/or a nucleic acid cargo entity comprising chemically modifies nucleotides provides stability to the nucleic acid cargo. This may be particular useful for guide RNA (e.g., for use in a Cas gRNA complex).

In some embodiments, a synthetic nucleic acid is an ASO or chemically modified RNA.

In some embodiments, a cargo entity may be a nucleic acid (e.g., DNA or RNA) or another molecule. Nucleic acid cargo entities can include, but are not limited to, DNA encoding a polypeptide of interest, mRNA, siRNA, shRNA, miRNA, an antisense oligonucleotide, and combinations thereof. Other potential cargo entities include, but are not limited to, viral and non-viral vectors (e.g., nucleocapsids) that are expressed inside a cell (or can be delivered to the cytosol of a cell), and ribonucleoprotein complexes, such as CRISPR-type entities and endogenous complexes such as DICER or RISC bound to natural or synthetic RNA such as miRNA, shRNA, etc.). In some embodiments, a cargo that can be expressed in a cell or physically delivered to the inside of a cell can be fused (genetically or synthetically) to an ABI protein/peptide or an ABA-binding sequence and is expressly contemplated here. Genetic fusion can be achieved by expressing in a lipid bilayer particle (e.g., a CDMP such as an EV) producing cell the two or more components of the chimeric polypeptide or peptide. Alternatively, the cargo entity fused to a loading domain may be delivered to a lipid bilayer particle (e.g., a CDMP such as an EV) producing cells leading to subsequent incorporation into the lipid bilayer particles. Alternatively, the cargo entity fused to a loading domain may be inserted into lipid bilayer particles after secretion from producer cells.

Additionally or alternatively, in some embodiments, the cargo entity may be a membrane bound cargo molecule. In some embodiments, the membrane bound cargo entity may comprise (i) a targeting peptide/protein and (ii) a transmembrane domain. Exemplary targeting peptides include but are not limited to any antibody fragments or antigen binding fragments, e.g., Fab, Fab′ and F(ab′)₂, Fd, scFv, single-chain antibodies, disulfide-linked Fvs (sdFv), and nanobodies. The targeting peptide (e.g., scFv) may bind to a target of interest on a specific cell type, such as a T cell. In some embodiments, the targeting peptide may be a Fab, Fab′ and F(ab′)₂, Fd, scFv, single-chain antibodies, disulfide-linked Fvs (sdFv), a de novo-designed binding molecule, affinibody, a DARPIN, or nanobody that binds to, for example, CD2 or another target that is associated with T cells. For example, other suitable targets include, but are not limited to, CD3, CD4, CD8, CD25, CD127, CD39, CD45RA, CTLA-4, and PD-1.

In some embodiments, a lipid bilayer particle targeting system of any one of the above embodiments further comprises abscisic acid (ABA), wherein the cargo-loading domain of the chimeric polypeptide and/or the ABA-binding sequence of the second chimeric polypeptide can bind to ABA, leading to dimerization of the chimeric polypeptide and the second chimeric polypeptide.

In some embodiments, a lipid bilayer particle encompasses or contains within it a viral nucleocapsid or derivatives thereof, a synthetic nucleic acid, a transcription factor, a recombinase, a base editor, prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, a ribonucleoprotein complex, or an antisense oligonucleotide.

Also disclosed herein are nucleic acids encoding a chimeric polypeptides disclosed herein. For example, in some embodiments, the cargo-loading domain of a chimeric loading polypeptide may be encoded by ATGACCAGAGTGCCCCTGTACGGCTTCACCAGCATTTGTGGCAGACGGCCCGAAAT GGAAGCCGCCGTGTCTACAATCCCCAGATTCCTCCAGAGCAGCAGCGGCTCCATGCT GGACGGCAGATTCGATCCTCAGAGCGCCGCTCACTTCTTCGGCGTGTACGATGGACA TGGCGGAAGCCAGGTGGCCAACTACTGCCGCGAAAGAATGCATCTGGCCCTGGCCG AGGAAATCGCCAAAGAAAAGCCCATGCTGTGCGACGGCGACACCTGGCTGGAAAA GTGGAAGAAGGCCCTGTTCAACAGCTTCCTGAGAGTGGACAGCGAGATCGAGAGCG TGGCCCCTGAAACAGTGGGCAGCACATCTGTGGTGGCCGTGGTGTTTCCCAGCCACA TCTTCGTGGCTAACTGCGGCGATAGCAGAGCCGTGCTGTGCAGAGGAAAAACAGCC CTGCCTCTGTCCGTGGACCACAAGCCTGATAGAGAGGATGAGGCCGCCAGAATTGA AGCCGCTGGCGGCAAAGTGATCCAGTGGAATGGCGCTAGAGTGTTCGGCGTGCTGG CCATGAGTAGATCCATCGGCGATAGATACCTGAAGCCTAGCATCATCCCCGATCCTG AAGTGACCGCCGTGAAGAGAGTGAAAGAGGACGACTGCCTGATCCTGGCCTCTGAC GGTGTCTGGGACGTGATGACAGATGAAGAGGCCTGCGAGATGGCCCGGAAGAGAAT CCTGCTGTGGCACAAGAAAAACGCCGTGGCCGGGGATGCTTCTCTGCTGGCTGACG AGAGAAGAAAAGAGGGCAAAGACCCCGCTGCCATGTCTGCCGCCGAGTACCTGTCT AAGCTGGCCATCCAGAGAGGCAGCAAGGACAACATCAGCGTGGTGGTCGTGGACCT GAAA (SEQ ID NO: 5), a variant nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to SEQ ID NO: 5, or a functional fragment thereof. In general, a fragment is considered a functional fragment if it encodes a protein or peptide that is capable of increasing active loading of a cargo entity to a lipid bilayer particle, binding to an ABI1-binding protein, or a combination thereof.

In some embodiments, the ABA-binding sequence of a second chimeric peptide may be encoded by ATGGGCGGAGGAGCCCCTACCCAGGACGAGTTCACCCAGCTGAGCCAGAGCATCGC TGAGTTCCACACCTACCAGCTGGGAAACGGACGCTGTTCCAGCCTGCTGGCACAGA GAATCCACGCTCCTCCTGAGACAGTGTGGAGTGTGGTGCGCAGATTCGACCGCCCTC AGATTTACAAGCACTTCATCAAGAGCTGCAACGTGAGCGAGGACTTCGAGATGAGA GTGGGATGTACCAGAGATGTGAACGTGATCAGCGGACTGCCTGCCAACACCAGCAG AGAGAGACTGGACCTGCTGGACGATGACCGCAGAGTGACCGGCTTCAGCATCACCG GAGGTGAGCACAGACTGAGAAACTACAAGAGCGTGACCACCGTCCACCGCTTCGAG AAGGAAGAGGAAGAGGAGCGCATCTGGACCGTGGTGCTGGAGAGCTACGTCGTGG ACGTGCCCGAGGGCAACAGCGAAGAGGATACCCGCCTGTTCGCTGACACCGTGATC AGACTGAACCTCCAGAAGCTGGCCAGCATCACCGAGGCAATGAAC (SEQ ID NO: 1), a variant nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to SEQ ID NO: 1, or a functional fragment thereof. In general, a fragment is considered a functional fragment if it encodes a protein or peptide that is capable of increasing active loading of the cargo entity to the lipid bilayer particle, binding to ABI1 or a variant or fragment thereof, or a combination thereof.

In some embodiments, the linker of any of the disclosed peptides may be encoded by one of SEQ ID NOs: 9 (ACTAGTGGCGGCGGAGGCAGCGGAGGCGGATCTGGCGGAGGATCT), 11 (ACGCGTGGCGGCGGAGGCAGCGGAGGCGGATCTGGCGGAGGATCT), or 13 (GGCGGCGGAGGAAGTGGCGGCGGATCTGGCGGAGGATCTACCGGT), or a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to one of SEQ ID NOs: 9, 11, or 13.

In some embodiments, a chimeric targeting polypeptides may optionally comprise a first cargo entity fused thereto. The first cargo entity is not particularly limited and may be any cargo entity disclosed herein. For instance, in some embodiments, the first entity may be an ABA-binding sequence comprising a pyrabactin resistance 1-like (PYL1) sequence. In some embodiments, the PYL1 sequence comprises residues 33-209 of wild type PYL1. In some embodiments, the PYL1 sequence comprises MGGGAPTQDEFTQLSQSIAEFHTYQLGNGRCSSLLAQRIHAPPETVWSVVRRFDRPQIY KHFIKSCNVSEDFEMRVGCTRDVNVISGLPANTSRERLDLLDDDRRVTGFSITGGEHRLR NYKSVTTVHRFEKEEEEERIWTVVLESYVVDVPEGNSEEDTRLFADTVIRLNLQKLASIT EAMN (SEQ ID NO: 2), TQDEFTQLSQSIAEFHTYQLGNGRCSSLLAQRIHAPPETVWSVVRRFDRPQIYKHFIKSCN VSEDFEMRVGCTRDVNVISGLPANTSRERLDLLDDDRRVTGFSITGGEHRLRNYKSVTT VHRFEKEEEEERIWTVVLESYVVDVPEGNSEEDTRLFADTVIRLNLQKLASITEAMN (SEQ ID NO: 3), or

(SEQ ID NO: 2)
MGGGAPTQDEFTQLSQSIAEFHTYQLGNGRCSSLLAQRIHAPPETVWSVV
RRFDRPQIYKHFIKSCNVSEDFEMRVGCTRDVNVISGLPANTSRERLDLL
DDDRRVTGFSITGGEHRLRNYKSVTTVHRFEKEEEEERIWTVVLESYVVD
VPEGNSEEDTRLFADTVIRLNLQKLASITEAMN,
(SEQ ID NO: 3)
TQDEFTQLSQSIAEFHTYQLGNGRCSSLLAQRIHAPPETVWSVVRRFDRP
QIYKHFIKSCNVSEDFEMRVGCTRDVNVISGLPANTSRERLDLLDDDRRV
TGFSITGGEHRLRNYKSVTTVHRFEKEEEEERIWTVVLESYVVDVPEGNS
EEDTRLFADTVIRLNLQKLASITEAMN,

or
a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 2 or 3, or
a functional fragment of SEQ ID NO: 2, SEQ ID NO: 3, or a variant amino acid sequence thereof. For the purposes of the present disclosure, a functional fragment of a ABA-binding sequence may be about 5 amino acids long, about 10 amino acids long, about 15 amino acids long, about 20 amino acids long, about 25 amino acids long, about 30 amino acids long, about 35 amino acids long, about 40 amino acids long, about 45 amino acids long, about 50 amino acids long, about 55 amino acids long, about 60 amino acids long, about 65 amino acids long, about 70 amino acids long, about 75 amino acids long, about 80 amino acids long, about 85 amino acids long, about 90 amino acids long, about 95 amino acids long, about 100 amino acids long, about 105 amino acids long, about 110 amino acids long, about 115 amino acids long, about 120 amino acids long, about 125 amino acids long, about 130 amino acids long, about 135 amino acids long, about 140 amino acids long, about 145 amino acids long, about 150 amino acids long, about 155 amino acids long, about 160 amino acids long, about 165 amino acids long, about 170 amino acids long, about 175 amino acids long, about 180 amino acids long, about 185 amino acids long, about 190 amino acids long, about 195 amino acids long, about 200 amino acids long, about 205 amino acids long, about 210 amino acids long, about 215 amino acids long, about 220 amino acids long, about 225 amino acids long, about 230 amino acids long, about 235 amino acids long, about 240 amino acids long, about 245 amino acids long, or about 250 amino acids long. In other words, a functional fragment may be 5-50 amino acids, 5-40 amino acids, 5-30 amino acids, 5-20 amino acids, 5-15 amino acids, 10-50 amino acids, 10-40 amino acids, 10-30 amino acids, or 10-20 amino acids. In general, a fragment is considered a functional fragment if it is capable of increasing active loading of the cargo entity to the lipid bilayer particle, binding to ABI1 or a variant or fragment thereof, or a combination thereof.

In another aspect, the present disclosure provides a nucleic acid encoding the chimeric peptide or the lipid bilayer particle targeting system of any one of the above embodiments.

In another aspect, the present disclosure provides a production cell comprising the targeting chimeric polypeptide, the lipid bilayer particle targeting system, the lipid bilayer particle (e.g., CDMP such as an extracellular vesicle), or the nucleic acid of any one of the above embodiments. In some embodiments, a production cell is a mammalian cell. Suitable mammalian cells include, but are not limited to, HEK293, HEK293FT, PER.C6, mesenchymal stem cells, megakaryocytes, iPSCs, T cells, erythrocytes and erythropoetic precursors, and iPSC-derived version of any of the preceding cells.

In another aspect, the present disclosure provides methods of producing a lipid bilayer particle that targets a recipient cell, such as an immune cell, comprising culturing a production cell of the foregoing aspect, and harvesting lipid bilayer particle produced by the production cell.

In some embodiment's, a cargo entity is an intraparticular macromolecular assembly (e.g., lentiviral cores, AAV particles, other viral cores, VLP cores, and subunits thereof).

In some embodiments, a cargo entity is a nucleocapsid. In some embodiments, a nucleocapsid is a viral nucleocapsid. In some embodiments, a nucleocapsid is a recombinant viral nucleocapsid. In some embodiments, a nucleocapsid comprises cargo nucleic acids and cargo polypeptides.

In some embodiments, a cargo entity is or encodes an AAV nucleocapsid, or a LVV nucleocapsid, or fragments thereof.

### Second Chimeric Polypeptide

In addition to chimeric targeting polypeptides described herein, in some embodiments, provided lipid bilayer particles may further comprise a second chimeric polypeptide (i.e., a chimeric loading polypeptide) comprising a cargo-loading domain comprising an abscisic acid-insensitive 1 (ABI1) sequence. In some embodiments, a second chimeric peptide may further comprise a linker that connects the cargo entity (e.g., cargo entity polypeptide) and the cargo-loading domain. The linker may comprise (1) an amino acid sequence selected from SEQ ID NO: 10 (TSGGGGSGGGSGGGS), SEQ ID NO: 12 (TRGGGGSGGGSGGGS), SEQ ID NO: 14 (GGGGSGGGSGGGSTG), SEQ ID NO: 15 (DQSNSEEAKKEEAKKEEAKKSNS), SEQ ID NO: 16 (SGGGSGGGSGGGSGGSGGSGGGSGGSGGSGGGSGGGSGGG), and SEQ ID NO: 17 (ESKYGPPAPPAP); or (2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 10, 12, 14, 15, 16, or 17.

The cargo-loading domain of the second chimeric peptide may be a truncated variant of a wild-type protein that comprises an extracellular vesicle targeting domain. For examples, the cargo-loading domain of the second chimeric peptide comprises residues 126-423 of wild type ABI1. In some embodiments, the cargo-loading domain of the second chimeric peptide comprises:

(SEQ ID NO: 6)
MTRVPLYGFTSICGRRPEMEAAVSTIPRFLQSSSGSMLDGRFDPQSAAHF
FGVYDGHGGSQVANYCRERMHLALAEEIAKEKPMLCDGDTWLEKWKKALF
NSFLRVDSEIESVAPETVGSTSVVAVVFPSHIFVANCGDSRAVLCRGKTA
LPLSVDHKPDREDEAARIEAAGGKVIQWNGARVFGVLAMSRSIGDRYLKP
SIIPDPEVTAVKRVKEDDCLILASDGVWDVMTDEEACEMARKRILLWHKK
NAVAGDASLLADERRKEGKDPAAMSAAEYLSKLAIQRGSKDNISVVVVDL
K,
(SEQ ID NO: 7)
VPLYGFTSICGRRPEMEAAVSTIPRFLQSSSGSMLDGRFDPQSAAHFFGV
YDGHGGSQVANYCRERMHLALAEEIAKEKPMLCDGDTWLEKWKKALFNSF
LRVDSEIESVAPETVGSTSVVAVVFPSHIFVANCGDSRAVLCRGKTALPL
SVDHKPDREDEAARIEAAGGKVIQWNGARVFGVLAMSRSIGDRYLKPSII
PDPEVTAVKRVKEDDCLILASDGVWDVMTDEEACEMARKRILLWHKKNAV
AGDASLLADERRKEGKDPAAMSAAEYLSKLAIQRGSKDNISVVVVDLK,

a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 6 or 7, or
a functional fragment of SEQ ID NO: 6, SEQ ID NO: 7, or a variant amino acid sequence thereof. For the purposes of the present disclosure, a functional fragment of ABI1 may be about 5 amino acids long, about 10 amino acids long, about 15 amino acids long, about 20 amino acids long, about 25 amino acids long, about 30 amino acids long, about 35 amino acids long, about 40 amino acids long, about 45 amino acids long, about 50 amino acids long, about 55 amino acids long, about 60 amino acids long, about 65 amino acids long, about 70 amino acids long, about 75 amino acids long, about 80 amino acids long, about 85 amino acids long, about 90 amino acids long, about 95 amino acids long, about 100 amino acids long, about 105 amino acids long, about 110 amino acids long, about 115 amino acids long, about 120 amino acids long, about 125 amino acids long, about 130 amino acids long, about 135 amino acids long, about 140 amino acids long, about 145 amino acids long, about 150 amino acids long, about 155 amino acids long, about 160 amino acids long, about 165 amino acids long, about 170 amino acids long, about 175 amino acids long, about 180 amino acids long, about 185 amino acids long, about 190 amino acids long, about 195 amino acids long, about 200 amino acids long, about 205 amino acids long, about 210 amino acids long, about 215 amino acids long, about 220 amino acids long, about 225 amino acids long, about 230 amino acids long, about 235 amino acids long, about 240 amino acids long, about 245 amino acids long, or about 250 amino acids long. In other words, a functional fragment may be 5-50 amino acids, 5-40 amino acids, 5-30 amino acids, 5-20 amino acids, 5-15 amino acids, 10-50 amino acids, 10-40 amino acids, 10-30 amino acids, or 10-20 amino acids. In general, a fragment is considered a functional fragment if it is capable of increasing active loading of the cargo entity to the lipid bilayer particle, binding to an ABI1-binding protein, or a combination thereof.

### Methods of Making and Using

Those skilled in the art, reading the present disclosure will appreciate its remarkable range of applicability. Provided engineered lipid bilayer particles may be suitable for therapeutic applications. In some embodiments, lipid bilayer particles or preparations provided herein can be contacted with recipient cells ex vivo (e.g., CAR-T) or in vivo (e.g., HIV).

Among other things, ability to effectively deliver cargo entities (e.g., polypeptide cargo entities, nucleic acid cargo entities, saccharides cargo entities, lipid cargo entities, small cargo entities, complex cargo entities, etc.) to T cells is useful, among other things, to provide engineered T cells, e.g., for use as T cell therapies (e.g., CAR-T therapies). Alternatively or additionally, ability to effectively deliver cargo entities is useful in treating HIV (e.g., by delivering cargo entities that inhibit, interrupt or destroy one or more aspect of HIV or its lifecycle). In some embodiments, cargo entities such as siRNAs, Cas9-sgRNA etc. are useful in targeting HIV.

In another aspect, the present disclosure provides a method of targeting a cargo entity to a recipient cell (e.g., an immune cell such as a lymphocyte) using a lipid bilayer particle (CDMP) such as EV). In general, a lipid bilayer particle will comprise a targeting chimeric peptide of any one of the above embodiments (e.g., a targeting chimeric peptide comprising a targeting domain). Thus, the present disclosure provides methods of targeting delivery of a cargo entity to a recipient cell (e.g., an immune cell, such as a lymphocyte), comprising administering to an individual a lipid bilayer particle as disclosed herein, wherein the lipid bilayer particle comprises a cargo entity.

In some embodiments, a cargo entity is a polypeptide cargo entity. In some embodiments, the cargo entity may a peptide, polypeptide, or protein of interest to be delivered to a recipient cell, such as an enzyme, a therapeutic agent (e.g., an antibody, inhibitor, an agonist, and an antagonist), or a fluorescent protein. In some embodiments, the cytosolic polypeptide cargo entity can be selected from any one or more of base editors, prime editors, TALENs, ZFNs, kinases, kinase inhibitors, activators or inhibitors of receptor-signaling, intrabodies, chromatin-modifying synthetic transcription factors, natural transcription factors, and mutant forms thereof. In some embodiments, the cytosolic polypeptide cargo entity is a CRISPR enzyme, e.g., a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is a Cas9 protein (e.g., a naturally-occurring bacterial Cas9 as well as any chimeras, mutants, homologs or orthologs). Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof.

In some embodiments, a cargo entity is a nucleic acid cargo entity. In some embodiments, the first or the second cargo entity may be a nucleic acid (e.g., DNA or RNA) or another molecule. Nucleic acid cargo entities can include, but are not limited to, DNA encoding a protein or peptide of interest, mRNA, siRNA, shRNA, miRNA, an antisense oligonucleotide, and combinations thereof. Other potential cargo entities include, but are not limited to, viral and non-viral vectors that are expressed inside a cell (or can be delivered to the cytosol of a cell), and ribonucleoprotein complexes, such as CRISPR-type entities and endogenous complexes such as DICER or RISC bound to natural or synthetic RNA such as miRNA, shRNA, etc.). Indeed, those skilled in the art will recognize that any cargo that can be expressed in a cell or physically delivered to the inside of a cell can be fused (genetically or synthetically) to an ABI protein/peptide or an ABA-binding sequence and is expressly contemplated here. Genetic fusion can be achieved by expressing in a lipid bilayer particle (e.g., a CDMP such as an EV) producing cell the two or more components of the chimeric polypeptide or peptide. Alternatively, the cargo entity fused to a loading domain may be delivered to a lipid bilayer particle (e.g., a CDMP such as an EV) producing cells leading to subsequent incorporation into the lipid bilayer particles. Alternatively, the cargo entity fused to a loading domain may be inserted into lipid bilayer particles after secretion from producer cells.

Thus, in some embodiments, the cargo entity is or comprises a viral nucleocapsid, a synthetic nucleic acid, a transcription factor, a recombinase, a base editor, a prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex.

In some embodiments, the cargo entity comprises a nucleic acid sequence encoding a chimeric antigen receptor.

The disclosed lipid bilayer particle may be formulated as part of a pharmaceutical composition for treating a disease or disorder and the pharmaceutical composition may be administered to a patient in need thereof to deliver the cargo entities to recipient cells (e.g., lymphocytes) in order to treat the disease or disorder. Additionally or alternatively, the lipid bilayer particle may provide to the recipient cell (e.g., lymphocyte) a nucleic acid sequence encoding a protein of interest, such as a chimeric antigen receptor (CAR), such that the CAR is expressed by the lymphocyte, as described in more detail below.

The present disclosure also provides ex vivo methods of targeting delivery of a cargo entity to a lymphocyte, comprising obtaining a population of lymophcytes from an individual, and contacting the population of lymphocytes ex vivo with the lipid bilayer particle as disclosed herein, wherein the lipid bilayer particle comprises a cargo entity. For the purposes of the ex vivo methods, all of the cargo entities mentioned above are similarly suitable and can be delivered to the population of recipient cells (e.g., immune cells, such as lymphocytes). In some embodiments, a population of lymphocytes were obtained via apheresis. In some embodiments of ex vivo methods, the methods may further comprise administering the population of recipient cells (e.g., immune cells, such as lymphocytes) back into the individual after the recipient cells (e.g., immune cells, such as lymphocytes) have been contacted with the lipid bilayer particle.

For example, a patient may undergo apheresis to isolate a population of recipient cells (e.g., immune cells, such as lymphocytes), which are then contacted with a lipid bilayer particle as disclosed herein, which contain a ribonucleoprotein complex and a nucleic acid encoding a chimeric antigen receptor (CAR). The ribonucleoprotein complex and nucleic acid encoding the CAR would be delivered into the recipient cell (e.g., lymphocyte), and the nucleic acid sequence would be incorporated into the genome and expressed by the recipient cell (e.g., lymphocyte), such that the CAR is expressed on the recipient cell (e.g., lymphocyte) surface. Expression of a CAR by a recipient cell (e.g., lymphocyte) could similarly be achieved in vivo by administering the CD2-targeted lipid bilayer particles to the patient (e.g., via intravenous injection or infusion).

### Production

In addition to targeting, the present disclosure also provides technologies (e.g., methods) for loading cargo into lipid bilayer particles and/or otherwise producing engineered lipid bilayer particles (e.g., cargo-loaded engineered lipid bilayer particles) as described herein.

In some embodiments, technologies for loading a cargo entity into a lipid bilayer particle (e.g., CDMP such as EV) may comprise expressing in a production cell (a) an nucleic acid (e.g., mRNA) that encodes a targeting chimeric polypeptide, a fusogen entity polypeptide and/or a cargo entity and (b) expressing in the production cell the targeting chimeric polypeptide and/or the fusogen entity polypeptide. In some embodiments, a production cell is a eukaryotic cell, The mRNA for the chimeric peptide optionally comprising the cargo entity and/or the fusogen entity polypeptide may be expressed from one or more vectors that are transfected into suitable production cells for producing the disclosed extracellular vesicles. Note that the vector may also be stably transfected. The vector or vectors for expressing the mRNA for the chimeric peptide comprising the cargo entity may be packaged in a kit designed for preparing the disclosed extracellular vesicles. In some embodiments, a kit comprises lipid bilayer particles with synthetic nucleic acids (such as antisense oligonucleotides) or plasmids.

In some embodiments, a production cell is a mammalian cell. In some embodiments, a mammalian cell is optionally selected from HEK293, HEK293FT, a mesenchymal stem cell, a megakaryocyte, an induced pluripotent stem cell (iPSC), a T cell, an erythrocyte, an erythropoetic precursor, and an iPSC-derived version of any of the preceding cells.

In some embodiments, loading of the cargo entity of the chimeric loading peptide is enhanced compared to passive cargo loading. For example, methods provided herein (e.g., comprising a lipid bilayer particle comprising a targeting chimeric polypeptide and/or a fusogen entity polypeptide) can achieve up to a 23-fold enrichment of cargo entities in microvesicles and up to a 49-fold enrichment in exosomes compared to passive loading in these respective particles.

In another aspect, the present disclosure provides technologies for (e.g., methods of) loading multiple (e.g., two) cargo entities into a lipid bilayer particle, such as a CDMP (e.g., an extracellular vesicle (EV)), comprising expressing in a production a cell lipid bilayer particle loading system (comprising a targeting chimeric polypeptide, fusion entity polypeptide, or both) of any one of the above embodiments.

In some embodiments, co-localization of the cargo entity of the chimeric polypeptide or peptide and the second cargo entity of the second chimeric polypeptide is enhanced compared to passive cargo loading.

In some embodiments, expression of a targeting chimeric polypeptide or a fragment thereof (e.g., a VLR) alongside a viral cargo entity (e.g., a standard lentiviral packaging plasmids) leads to viral genomic titers incurring no more than 0% loss, or no more than 10% loss, nor more than 20% loss, not more than 30% loss, not more than 40% loss, or no more than 50% loss. In some such embodiments, a standard lentiviral packaging plasmids may include a fusogen (e.g., VSV-G) and viral helper plasmids. This is comparable to expression of a viral cargo entity (e.g., a standard lentiviral packaging plasmids) without expression of a targeting chimeric polypeptide or a fragment thereof (e.g., a VLR) where loss is observed.

### Lymphocyte Targeting

Certain embodiments of provided technologies are particularly useful or beneficial in achieving lymphocyte targeting (e.g., to human lymphocyte cells or populations thereof).

Those skilled in the art are aware that CRISPR-Cas9 mediated genome engineering of human T cells is an area of active investigation for the development of therapeutics to treat cancer, autoimmunity, and infectious disease. Delivery of the programmable nuclease Cas9 with a single guide RNA (sgRNA) complementary to a target sequence results in the introduction of double-stranded breaks that can introduce frameshift mutations in coding genes and the ablation of protein expression. Alternately, co-delivery of a homology-directed repair template can insert specified mutations, insertions, or deletions into the genomes of recipient cells. While this technology has multiple applications, translation of this strategy remains difficult, at least in part due to the challenges associated with in vivo delivery of Cas9. One approach that leverages foundational gene therapy advances is adeno-associated virus (AAV) vehicles, although safety and efficacy are often limited by anti-vector immunity and limited tissue tropism. Virus-like particles (VLPs) can also deliver Cas9 nucleases or base editors, although it remains unclear whether the immunogenicity of viral proteins will likewise limit these approaches. Synthetic nanoparticle-nucleic acid (e.g., mRNA) delivery is an alternative to viral vectors and has been successfully used for in vivo delivery of mRNA to confer sustained expression of chimeric antigen receptors in murine T cells. However, achieving efficient and specific T cell targeting in a manner that confers the transient expression of Cas9 needed to avoid off-target effects remains challenging. These general difficulties are uniquely compounded by the challenge of delivering any cargo to T cells, which exhibit low rates of endocytosis. Altogether, there exists substantial opportunity to improve delivery systems that could enable delivery of biologics to T cells inside a patient.

A promising emerging strategy is the use of extracellular vesicles (EVs) to deliver biomolecular cargo. EVs are nanoscale, membrane-enclosed particles secreted by all cells and naturally encapsulate proteins and nucleic acids during biogenesis. EVs mediate intercellular communication, delivering their contents to recipient cells to affect cellular function. Intrinsic properties such as non-toxicity and non-immunogenicity, as well as the ability to engineer surface and luminal cargo loading, make EVs an attractive platform for delivering a wide range of therapeutics. Cargo can be incorporated into vesicles either by overexpressing the cargo in the producer cells such that it is loaded during EV biogenesis or by physically or chemically modifying vesicles post-harvest. Cells that are genetically engineered to produce functionalized EVs may even be implanted to continuously generate such particles in situ. While modification of EVs post-harvest may confer cargo loading flexibility, this approach requires more extensive purification and introduces challenges from a manufacturing and regulatory standpoint.

Several recent studies have investigated the use of EVs to deliver Cas9 for treatment of cancer, hepatitis B, and genetic diseases, highlighting the promise of this method for achieving intracellular Cas9 delivery. However, many exploratory studies have employed EV engineering methods known to introduce artifacts in downstream experiments, which obscures how functional effects may be attributable to EVs. Of particular concern are methods that rely on transfecting EV producer cells with lipoplexes, loading EVs with electroporation methods known to result in cargo aggregation, or isolating EVs with commercial kits not intended for functional delivery applications, which have all been shown to introduce artifacts.²⁴⁻²⁶

Here, we address this need by developing an integrated bioengineering strategy for genetically engineering cells to direct the self-assembly and production of multifunctional EVs. As a motivating application, we systematically evaluate, compare, and generate techniques enabling EV targeting, active loading of protein cargo into EVs, and EV fusion to achieve functional cargo delivery to T cells. This exploration identifies key limitations and drivers of functional EV-mediated delivery, including a potential mechanism of receptor binding-mediated delivery enhancement to T cells. We validate these technologies by demonstrating a therapeutically relevant capability—delivering Cas9 ribonucleoprotein complex (RNP) to ablate the gene encoding the HIV co-receptor CXCR4 in primary human CD4⁺ T cells.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Methods and Materials

Plasmid construction. Plasmids were constructed using standard molecular biology techniques. Codon optimization was performed using the GeneArt gene synthesis tool (Thermo Fisher). PCR was performed using Phusion DNA polymerase (New England Biolabs, NEB), and plasmid assembly was performed via restriction enzyme cloning. Plasmids were transformed into TOP10 competent E. Coli (Thermo Fisher) and grown at 37° C.

Plasmid backbones. A modified pcDNA3.1 (Thermo Fisher V87020), was used to generate a general expression vector. Briefly, the hygromycin resistance gene and SV40 promoter were removed, leaving the SV40 origin of replication and poly(A) signal intact. The BsaI sites in the AmpR gene and 5′-UTR and the BpiI site in the bGH poly(A) signal were mutated. The lentiviral vector pGIPZ (Open Biosystems) was obtained through the Northwestern High Throughput Analysis Laboratory. PlentiCRISPRv2 was a gift from Feng Zhang (Addgene plasmid No. 52961).

Plasmid source vectors. Fluorescent proteins enhanced blue fluorescent protein 2 (EBFP2), enhanced yellow fluorescent protein (EYFP), and dimeric tomato (dTomato) were sourced from Addgene vectors (plasmid Nos. 14893, 58855, and 18917, respectively) gifted by Robert Campbell, Joshua Leonard, and Scott Sternson. Monomeric teal fluorescent protein 1 (TFP1) was synthesized by Thermo Fisher. The scFv was synthesized from a previously published scFv sequence derived from monoclonal antibody 9.6, and the PDGFR transmembrane domain was sourced from a pDisplay system vector (Addgene plasmid No. 61556, gifted by Robert Campbell). The C1C2 domain sequence was provided by Natalie Tigue and synthesized by Thermo Fisher. Constitutively active Cx43 and SLAM were synthesized by Thermo Fisher from Uniprot sequences P17302 CXA1_HUMAN and Q13291-1 SLAFl_HUMAN isoform 1, respectively. Plasmids encoding the measles virus glycoproteins were gifts from Isabelle Clerc, Thomas Hope, and Richard D'Aquila. pX330 encoding Cas9 was gifted by Erik Sontheimer (UMass), originally sourced from Addgene plasmid No. 42230 gifted by Feng Zhang. The CXCR4 sgRNA sequence was provided by Judd Hultquist and is as follows: GAAGCGTGATGACAAAGAGG (SEQ ID NO: 43). ABI and PYL domains were synthesized by Thermo Fisher and IDT, respectively.

Plasmid preparation. Bacteria were grown overnight in 100 mL LB+Amp cultures for 12-14 h. Cultures were spun at 3,000 g for 10 min to pellet the bacteria, and pellets were resuspended and incubated for 30 min in 4 mL of 25 mM Tris pH 8.0, 10 mM EDTA, 15% sucrose, and 5 mg/mL lysozyme. Bacteria were lysed for 15 min in 8 mL of 0.2 M NaOH and 1% SDS, followed by a 15 min neutralization in 5 mL of 3 M sodium acetate (pH 5.2). The precipitate was pelleted at 9,000 g for 20 min, and supernatant was filtered through cheese cloth and incubated for 1-3 h at 37° C. with 3 μL of 10 mg/mL RNAse A (Thermo Fisher). Samples were extracted with 5 mL phenol chloroform, and the aqueous layer was recovered after centrifugation at 7,500 g for 20 min. A second phenol chloroform extraction was performed with 7 mL solvent. 0.7 volumes isopropanol was added to the recovered supernatant, and samples were inverted and incubated at room temperature for 10 min prior to centrifugation at 9,000 g for 20 min to pellet the DNA mixture. Pellets were briefly dried and resuspended in 1 mL of 6.5% PEG 20,000 and 0.4 M NaCl. DNA was incubated on ice overnight and pelleted at 21,000 g for 20 min. The supernatant was removed, and pellets were washed in cold absolute ethanol and dried at 37° C. before resuspension in TE buffer (10 mM Tris, 1 mM EDTA, pH 8.0). DNA was diluted to 1 μg/μL using a Nanodrop 2000 (Thermo Fisher).

Cell culture. HEK293FT cells (Thermo Fisher R70007) were cultured in Dulbecco's Modified Eagle Medium (DMEM, Gibco 31600-091) supplemented with 10% FBS (Gibco 16140-071), 1% penicillin-streptomycin (Gibco 15140-122), and 4 mM additional L-glutamine (Gibco 25030-081). Jurkat T cells (ATCC TIB-152) were cultured in Roswell Park Memorial Institute Medium (RPMI 1640, Gibco 31800-105) supplemented with 10% FBS, 1% pen-strep, and 4 mM L-glutamine. Sublines generated from these cell lines were cultured in the same way. Cells were subcultured at a 1:5 or 1:10 ratio every 2-3 d, using Trypsin-EDTA (Gibco 25300-054) to remove adherent cells from the plate. Lenti-X cells (Takara) were cultured the same way with additional 1 mM sodium pyruvate (Thermo Fisher 11360-070). Primary human CD4⁺ T cells were cultured in RPMI supplemented with 10% FBS, 1% pen-strep, 5 mM HEPES, 5 mM sodium pyruvate, and 20 U/mL IL-2 (added fresh at time of use). Cells were maintained at 37° C. at 5% CO₂. HEK293FT and Jurkat cells tested negative for Mycoplasma with the MycoAlert Mycoplasma Detection Kit (Lonza LT07-318).

Transfection. For transfection of HEK293FT cells and derived cell lines in 15 cm dishes for EV packaging, cells were plated at a density of 18×10⁶ cells/dish (1×10⁶ cells/mL) 6-12 h prior to transfection. Cells were transfected with 30 μg DNA plus 1 μg of a fluorescent transfection control via the calcium phosphate method. Plasmid DNA was mixed with 2 M CaCl₂) (final concentration 0.3 M) and added to a 2×HEPES-buffered saline solution (280 mM NaCl, 0.5 M HEPES, 1.5 mM Na₂HPO₄) dropwise in a 1:1 ratio and mixed seven times by pipetting. The transfection solution was incubated for 3 min, mixed eight times by pipetting, and added gently to the side of the plate. For transfection of HEK293FT cells in 10 cm dishes for EV packaging, cells were plated at a density of 5×10⁶ cells/dish (6.25×10⁵ cells/mL) and transfected with 20 μg DNA plus 1 μg transfection control as described above, adding transfection mixture dropwise to the dish. Lenti-X cells were transfected in 10 cm dishes in the same manner, though were plated 24 h prior to transfection as per the manufacturer recommendation (Takara). For transfection of HEK293FT cells in 24 well plates, cells were plated at a density of 1.7×10⁵ cells/well (3.4×10⁵ cells/mL) and transfected with 200 μg DNA as described above, adding transfection mixture dropwise to the well. Medium was changed 12-16 h later. Jurkat lipofectamine transfections were performed according to the manufacturer's protocol.

Cell line generation. To generate lentivirus, HEK293FT or Lenti-X cells were plated in 10 cm dishes at a density of 5×10⁶ cells/dish (6.25×10⁵ cells/mL). 6-12 h later for HEK293FT or 24 h later for Lenti-X, cells were transfected with 10 μg of viral vector, 8 μg psPAX2, and 3 μg pMD2G via calcium phosphate transfection as described above. Medium was changed 12-16 h later. 28 h post media change, lentivirus was harvested from the conditioned medium. Medium was centrifuged at 500 g for 2 min to clear cells, and the supernatant was filtered through a 0.45 μm pore filter (VWR). Lentivirus was concentrated from the filtered supernatant by ultracentrifugation in Ultra Clear tubes (Beckman Coulter 344059) at 100,420 g at 4° C. in a Beckman Coulter Optima L-80 XP ultracentrifuge using an SW41Ti rotor. Supernatant was aspirated, leaving virus in ˜100 μL final volume, and concentrated lentivirus was left on ice for at least 30 min prior to resuspension, then used to transduce ˜1×10⁵ cells, either plated at the time of transduction or the day before. When appropriate, drug selection began 2 d post transduction, using antibiotic concentrations of 1 μg/mL puromycin (Invitrogen ant-pr) and 10 μg/mL blasticidin (Alfa Aesar J61883) on HEK293FT cells or 0.2 μg/mL puromycin and 2 μg/mL blasticidin on Jurkat cells. Cells were kept in antibiotics for at least two weeks with subculturing every one to two days.

Sorting of Cas9 reporter lines. Cells were prepared for fluorescence-activated cell sorting (FACS) by resuspending in either DMEM or RPMI, as appropriate, supplemented with 10% FBS, 25 mM HEPES, and 100 μg/mL gentamycin (Amresco 0304) at a concentration of 1×10⁷ cells/mL. Cells were sorted for the highest mTFP1 expressors (top 10% or less) lacking any dTomato expression on a BD FacsAria Ilu using a 488 nm laser (530/30 filter) and a 562 nm laser (582/15 filter). Cells were collected in DMEM or RPMI, as appropriate, supplemented with 20% FBS, 25 mM HEPES, and 100 μg/mL gentamycin. Cells were spun down and resuspended in normal growth medium with 100 μg/mL gentamycin for recovery.

EV production, isolation, and characterization. EV producer cell lines were plated in 10 or 15 cm dishes and transfected the same day by the calcium phosphate method where appropriate. Medium was changed to EV-depleted medium the following morning. EV-depleted medium was made by supplementing DMEM with 10% exosome depleted FBS (Gibco A27208-01), 1% pen-strep, and 4 mM L-glutamine. EVs were harvested from the conditioned medium 24-36 h post medium change by differential centrifugation as previously described.^{41,42} Briefly, conditioned medium was cleared of debris by centrifugation at 300 g for 10 min to remove cells followed by centrifugation at 2,000 g for 20 min to remove dead cells and apoptotic bodies. Supernatant was centrifuged at 15,000 g for 30 min in a Beckman Coulter Avanti J-26XP centrifuge with a J-LITE JLA 16.25 rotor to pellet microvesicles. Supernatant was collected and exosomes pelleted by ultracentrifugation at 120,416 g for 135 min in a Beckman Coulter Optima L-80 XP ultracentrifuge with an SW41 Ti rotor, using polypropylene ultracentrifuge tubes (Beckman Coulter 331372). All centrifugation steps were performed at 4° C. EV pellets were left in ˜100-200 μL of conditioned media and incubated on ice for at least 30 min after supernatant removal before resuspension. EV concentration was determined by NanoSight analysis. Samples were diluted in PBS to concentrations on the order of 10⁸ particles/mL for analysis. NanoSight analysis was performed on an NS300 (Malvern), software version 3.4. Three 30 s videos were acquired per sample using a 642 nm laser on a camera level of 14, an infusion rate of 30, and a detection threshold of 7. Default settings were used for the blur, minimum track length, and minimum expected particle size. EV concentrations were defined as the mean of the concentrations calculated from each video. Size distributions were generated by the software. For TEM, samples were fixed for 10 min in Eppendorf tubes by adding 65 μL of 4% PFA to 200 μL of EVs. 15 μL of fixed suspension was pipetted onto a plasma cleaned (PELCO easiGlow), formvar/carbon coated grid (EMS 300 mesh). After 10 min, the solution was removed by wicking with a wedge of filter paper, then washed by inverting the grid onto a drop of buffer for 30 seconds twice, followed with diH₂O once. A 2% uranyl acetate (Ted Pella) stain was applied twice and wicked after 30 s. Grids were allowed dry before storing in a grid box until use. Grids were imaged in a JEOL JEM 1230 TEM (JEOL USA) with a 100 KV accelerating voltage. Data was acquired with a Orius SC1000 CCD camera (Gatan). EVs were stored on ice and used within 10 days or stored at −80° C. for long term preservation.

Immunoblotting. For western blot analysis, cells were lysed in RIPA buffer (150 mM NaCl, 50 mM Tris-HCl pH 8.0, 1% Triton X-100, 05% sodium deoxycholate, 0.1% SDS, and one protease inhibitor cocktail tablet (Pierce PIA32953) per 10 mL) and incubated on ice for 30 min. Lysates were cleared by centrifugation at 12,000 g for 20 min at 4° C., and supernatant was harvested. Protein concentration was determined by BCA assay (Pierce) according to the manufacturer's instructions. Samples were normalized by protein content ranging from 1 to 2 μg (for cell lysates) or by vesicle count ranging from 1×10⁷ to 6×10⁸ (for EVs). Samples were heated in Laemmli buffer (60 mM Tris-HCl pH 6.8, 10% glycerol, 2% SDS, 100 mM dithiothreitol, 0.01% bromophenol blue) at 70° C. (for membrane-bound scFv and calnexin) or 95° C. (for Cas9, CD9, CD81, and Alix) for 10 min. Samples were loaded onto 4-15% polyacrylamide gradient Mini-PROTEAN TGX precast protein gels (Bio-Rad) and run at 50 V for 10 min followed by 100 V for 1 h. Protein was transferred to a PVDF membrane (Bio-Rad) at 100 V for 45 min. For anti-FLAG blots, membranes were blocked in 3% milk in TBS (50 mM Tris, 138 mM NaCl, 2.7 mM KCl, pH 8.0) for 30 min. Membranes were washed once in TBS for 5 min, then incubated in primary anti-FLAG antibody (Sigma F1804) diluted 1:1000 in 3% milk in TBS overnight at 4° C. Membranes were washed once for 5 min in TBS and twice in TBST 1 (50 mM Tris, 138 mM NaCl, 2.7 mM KCl, 0.05% Tween 20, pH 8.0) for 5 min each prior to secondary antibody staining. For all other blots, membranes were blocked in 5% milk in TBST 2 (50 mM Tris, 150 mM NaCl, 0.1% Tween 20, pH 7.6) for 1 h. Membranes were incubated in primary antibody diluted in 5% milk in TBST 2 overnight at 4° C. Primary antibodies include anti-HA (Cell Signaling Technology 377245 C29F4, 1:1000), anti-CD9 (Santa Cruz Biotechnology sc-13118, 1:500), anti-CD81 (Santa Cruz Biotechnology sc-23962, 1:500, run in non-reducing conditions), anti-Alix (Abcam Ab117600, 1:500), and anti-calnexin (Abcam Ab22595, 1:1000). Membranes were washed three times in TBST 2 for 5 min each prior to secondary antibody staining. HRP-conjugated anti-mouse (Cell Signaling Technology 7076) and anti-rabbit (Invitrogen 32460) secondary antibodies were diluted 1:3000 in 5% milk in TBST 2. Membranes were incubated in secondary antibody at room temperature for 1 h, then washed three times in TBST 2 (5 min washes). Membranes were probed with Clarity Western ECL Substrate (Bio-Rad) and either exposed to film, which was developed and scanned, or imaged using an Azure c280 imager. Images were cropped using Adobe Illustrator. No other image processing was employed.

Surface immunoblotting. Cells were transferred to FACS tubes (adherent cells were harvested using FACS buffer (PBS pH 7.4 with 0.05% BSA and 2 mM EDTA) prior to staining) with 1 mL of FACS buffer and centrifuged at 150 g for 5 min. Supernatant was decanted, and cells were resuspended in 50 μL of FACS buffer. 10 μL of human IgG (Thermo Fisher 027102) was added, cells were flicked to mix, and were incubated at 4° C. for 5 min. Conjugated primary antibody was then added at the manufacturer's recommended dilution, cells were flicked to mix and incubated at 4° C. for 30 min. Cells were then washed three times with 1 mL of FACS buffer, centrifuging at 150 g for 5 min and decanting the supernatant after each wash. Cells were resuspended in two drops of FACS buffer prior to flow cytometry. For Miltenyi Biotec antibodies, cells were stained at 4° C. for 15 min without blocking and were washed once prior to flow cytometry, as per manufacturer protocol. Antibodies used in this study were as follows: Anti-FLAG-APC (Abcam ab72569), anti-CD2-APC (R&D Systems FAB18561A), anti-CD25-PE (Miltenyi REA945, 130-115-628), anti-SLAM-PE (Miltenyi REA151, 130-123-970) and anti-mouse IgG1-APC (R&D Systems IC002A) or anti-human IgG1-PE (Miltenyi REA293, 130-113-438) were used as isotype controls where appropriate.

EV binding and uptake experiments. Jurkat T cells or primary human CD4⁺ T cells were incubated with EVs at an EV to cell ratio of 100,000:1 (typically 1×10¹⁰ EVs per 1×10⁵ cells) unless otherwise indicated. For Jurkats, cells were plated in a 48 well plate with 300 μL total volume. For primary T cells, cells were plated in a 96 well plate with 200 μL total volume. Cells were plated at the time of EV addition, and wells were brought to the appropriate volume with RPMI. For binding experiments, cells were incubated for 2 h at 37° C. unless otherwise indicated, then washed three times in FACS buffer, centrifuging at 150 g for 5 min for Jurkat cells or 400 g for 3 min for primary T cells. Cells were resuspended in one drop of FACS buffer prior to flow cytometry. To adsorb EVs to aldehyde/sulfate latex beads (Thermo Fisher), EVs were mixed with beads at a ratio of 1×10⁹ EVs per 2 μL beads diluted 1:10 in PBS. Volumes were normalized across samples with PBS, and beads and EVs were incubated for 15 min at room temperature. Samples were then brought to 200 μL with PBS and allowed to incubate for 2 h at room temperature while rocking. Cells were blocked with an anti-CD2 antibody binding the same epitope as the scFv (Beckman Coulter A60794) or with blank EVs for 1 h at 37° C. prior or EV incubation where indicated. For EV uptake experiments with viral glycoproteins, cells were incubated with EVs for 16 h at 37° C. To prepare for analysis, cells were washed twice in PBS, incubated with two drops of trypsin-EDTA for 5 min at 37° C. to remove surface bound vesicles. Cells were washed with RPMI to quench the trypsin, then washed twice more with FACS buffer prior to analysis.

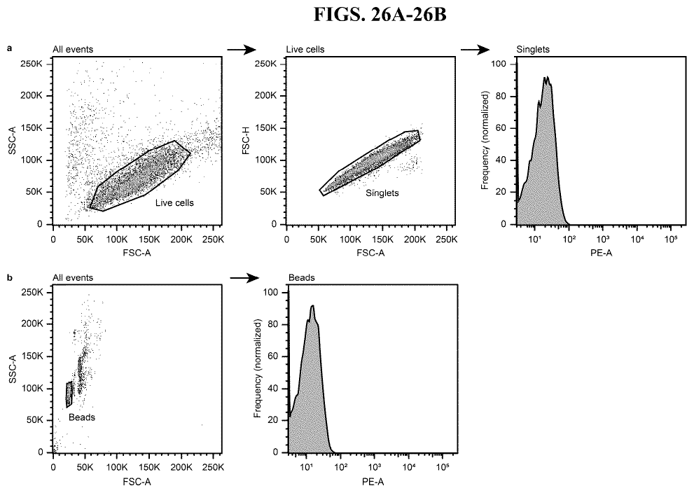

Analytical flow cytometry and analysis. Flow cytometry was performed on a BD LSR Fortessa Special Order Research Product using the 562 nm laser for dTomato (582/15 filter), the 488 nm laser for EYFP (530/30 filter), and the 488 nm and 405 nm lasers for mTFP1 (530/30 filter and 525/50 filter, respectively). Approximately 10,000 live cells were collected per sample for analysis. Data were analyzed using FlowJo v10 (FlowJo, LLC). Briefly, cells were identified using an FSC-A vs SSC-A plot and gated for singlets using an FSC-A vs FSC-H plot (FIGS. 26A-26B). Fluorescence data were compensated for spectral bleed-through where appropriate. Mean fluorescence intensity (MFI) of single-cell samples was exported and averaged across three biological replicates. Autofluorescence from untreated cells was subtracted from other samples. Standard error of the mean was propagated through calculations. Where indicated, 9 peak Ultra Rainbow Calibration Particles (Spherotech URCP-100-2H) were used to generate a calibration curve to convert fluorescence into absolute fluorescence units.

Confocal microscopy. Cells were transfected via the calcium phosphate method on poly L-lysine coated glass coverslips and mounted on glass slides for imaging. Microscopy images were taken on Leica SP5 II laser scanning confocal microscope using a 100× oil-immersion objective. Bright-field images were acquired at a PMT setting of 443.0 V. A 514 nm laser at 20% intensity and 94% smart gain was used for fluorescence excitation. Emission spectra were captured from 520-540 nm using an HyD sensor. Images were captured at 512×512 resolution at scanning speed of 400 Hz. Pseudocolored fluorescence images were contrast-adjusted in ImageJ such that 4% of pixels were saturated.

Affinity chromatography. Affinity chromatography isolation was performed as previously reported.⁵¹ Briefly, an anti-FLAG affinity column was prepared by loading anti-FLAG M2 affinity gel (Sigma A2220-1ML) in a 4 mL 1×5 cm glass column (Bio-Rad) and drained via gravity flow. The column was washed with 5 mL TBS (50 mM Tris-HCl, 150 mM NaCl, pH 7.5) and equilibrated with three sequential 1 mL washes with regeneration buffer (0.1 M glycine-HCl, pH 3.5), followed by a 5 mL wash of TBS. Concentrated EVs were loaded onto the top of the column and chased with 1-2 mL of TBS. The column was incubated with EVs for 5 min before continuing. The flow through was then re-loaded onto the column such that the EV-containing medium passed through the matrix five times. The column was washed with 10 mL TBS prior to elution. EVs were eluted with 2.5 mL elution buffer (100 μg/mL 3×FLAG peptide (Sigma F4799-4MG) in TBS), which was incubated on the column for 5-10 min after the void fraction was drained (˜1 mL). Five fractions of EVs were collected in 0.5 mL fractions (approximately 8 drops off the column per fraction). The column was regenerated with three sequential 1 mL washes with regeneration buffer and stored at 4° C. in storage buffer (50% glycerol, 0.02% sodium azide in TBS).

Cas9 in vitro cleavage assays. EVs were produced as described above with components transiently transfected in 10 cm dishes with the following DNA ratios: 6 μg anti-CD2 scFv, 9 μg Cas9 vector, 5 μg sgRNA vector, and 1 μg mTFP1 transfection control. EVs were lysed by incubation with mammalian protein extraction reagent (MPER, Thermo Fisher) for 10 min at room temperature (20-23° C.) with gentle agitation. 200 ng of linearized target plasmid template was added to vesicles with Cas9 reagent buffer (IDT, Alt-R CRISPR-Cas9 System), and samples were incubated at 37° C. for 1 h. Proteinase K (Thermo Fisher) was added to samples at 1 μL per 10 μL of reaction mixture and incubated at 55° C. for 10 min. Samples were run on a 1% agarose gel stained with SYBR safe (Thermo Fisher) and imaged using a BioDoc-It imaging system (VWR).

Primary CD4⁺ T cell isolation, culture, and activation. A surprising finding is that CD2 engagement, by either recombinant antibody or EV-displayed antibody, enhanced functional exosome-mediated delivery in vitro, though no comparable benefit for microvesicle-mediated delivery was observed. This combination of effects is not explained by known features of CD2/T cell biology, although it could be related to findings that ligand engagement triggers CD2 internalization. While scFv-displaying vesicles of both types specifically bound CD2 and were internalized to some degree, there may exist a difference in intracellular trafficking and fusion between the two vesicle populations. For example, CD2-binding-mediated trafficking might favor fusion over native uptake pathways in a way that differentially favors exosomes. This phenomenon warrants further study to elucidate underlying mechanisms.

EV functional delivery experiments. EVs were produced as described above with components transiently transfected in 10 cm dishes with the following DNA ratios: 6 μg anti-CD2 scFv, 9 μg dual Cas9 and sgRNA vector, either 2.5 μg each of measles virus glycoproteins H/F or 3 μg VSV-G with 2 μg filler promoterless pcDNA, and 1 μg mTFP1 transfection control. For generation of vesicles lacking the scFv, a PDGFR-bound 3×FLAG tag construct in the same vector backbone was transfected at the same plasmid copy number in place of the scFv. EVs were delivered to primary human CD4⁺ T cells as described above. Cells were cultured in the presence of EVs for 6 days, adding fresh supplemental RPMI and IL-2 every 2-3 days. For repeat dose administration, 100 μL of media were carefully removed from the top of each well and replaced with 100 μL fresh EVs and media. Cells were harvested on day 6 and washed with PBS by centrifugation at 400 g for 3 min a 4° C. to pellet. Cells were resuspended in 100 μL QuickExtract DNA Extract Solution (Lucigen QE9050), and genomic DNA was harvested according to the manufacturer's protocol. Briefly, samples were vortexed for 15 s, heated at 65° C. for 6 min, vortexed for 15 s, and heated at 98° C. for 2 min. DNA was stored at −80° C.

High Throughput Sequencing (NGS) library generation. Approximately 100 ng genomic DNA was used as a template in the first round PCR amplification. The CXCR4 region of interest was amplified with the following primers: F1: 5′ ACA CTC TTT CCC TAC ACG CTC TTC CGA TCT NNN NNG AGA AGC ATG ACG GAC AAG TAC AG 3′ (SEQ ID NO: 44) R1: 5′ GTG ACT GGA GTT CAG ACG TGT GCT CTT CCG ATC TNN NNN TCC CAA AGT ACC AGT TTG CCA C 3′ (SEQ ID NO: 45) The PCR protocol was as follows: 98° C. 3 min, (98° C. 15 s, 65° C. 30 s, 72° C. 3 s)×15, 72° C. 5 min, 4° C. 5 min. PCR products were purified using MagJET beads (Thermo Fisher K2821) and used as templates in a second round PCR amplification with the following primers: F2: 5′ AAT GAT ACG GCG ACC GAG ATC TAC ACT CTT TCC CTA CAC GAC GCT CTT CCG ATC T 3′ (SEQ ID NO: 46) R2: 5′ CAA GCA GAA GAC GGC ATA CGA GAT-Index-GTG ACT GGA GTT CAG ACG TGT GCT C 3′ (SEQ ID NO: 47) The PCR cycles were as follows: 98° C. 3 min, (98° C. 15 s, 69° C. 30 s, 72° C. 5 s)×20, 72° C. 5 min, 4° C. 5 min. PCR products were again purified using MagJET beads prior to HTS. Both first and second round PCRs were run with primer concentrations of 200 nM and Phusion DNA polymerase.

HTS. Genomic DNA sample concentrations were measured on a Qubit using an HS dsDNA kit and pooled in libraries with equimolar concentrations. Libraries were diluted to 4 nM in serial dilutions. Libraries and PhiX were denatured with NaOH according to the Illumina MiSeq guide and diluted to 14 pM. Reaction mixtures consisted of 8% PhiX and 92% library. Samples were run on an Illumina MiSeq using a MiSeq Reagent Kit v3, collecting paired-end reads. Data were analyzed using custom code developed by 496code.

Statistical analysis. Statistical details are described in the figure legends. Unless otherwise stated, three independent biological replicates (cells) or technical replicates (beads) were analyzed per condition, and the mean fluorescence intensity of approximately 10,000 live single cells or beads were analyzed per sample. Unless otherwise indicated, error bars represent the standard error of the mean. Pairwise comparisons were made using two-tailed Student's t-tests in Excel with the null hypothesis that the two samples were equal. The significance threshold was set to 0.05. Tests were followed by a Benjamini-Hochberg procedure applied within each panel of a given figure to decrease the false discovery rate.

### Example 2: Strategy for Engineering Multifunctional EVs for Achieving Delivery to T Cells

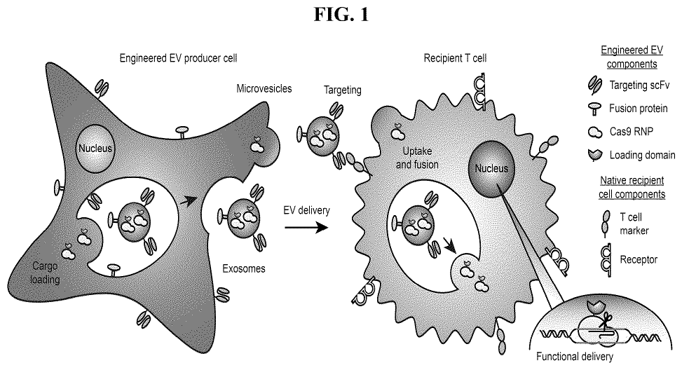

Our overall approach for developing technologies toward the goal of enabling targeted delivery of biomolecules to T cells is to address each limiting step in the process (FIG. 1) cargo loading into EVs during biogenesis, binding of EVs to specific recipient cells, uptake, and fusion of the EV with a recipient cell to release cargo into the T cell cytoplasm. Our approach relies entirely upon genetically-encodable functions, and we term this strategy GEMINI Genetically Encoded Multifunctional Integrated Nanovesicles.

### Example 3: Engineered Membrane Scaffolds Display scFvs on EVs

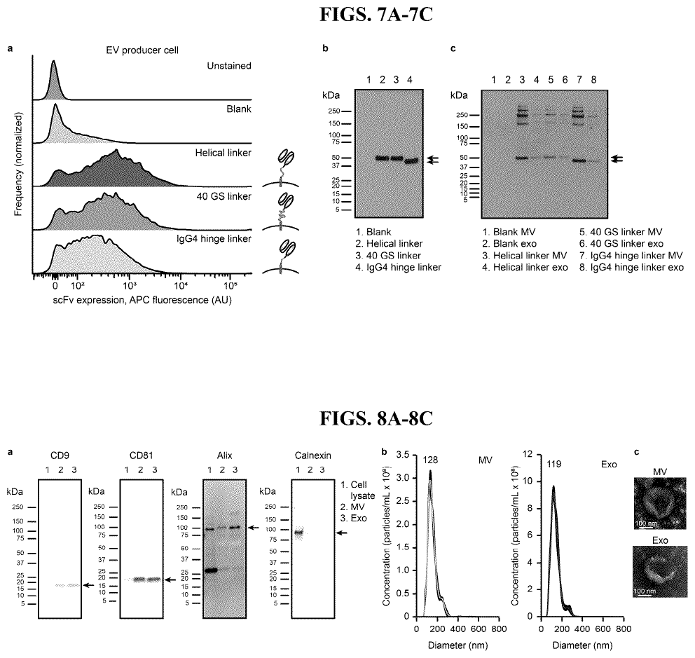

We first investigated strategies for conferring EV targeting. To promote specific interactions between EVs and recipient cells and facilitate EV uptake, a promising strategy is displaying targeting moieties on the EV surface. This strategy was pioneered using display of small peptides, although we and others have demonstrated that these effects are modest and variable.³⁴ Recently, display of high affinity targeting domains, including nanobodies and antibody single chain variable fragments (scFvs), conferred EV targeting to receptors such as EGFR and HER2. In these cases, scFv display was achieved by fusion to the C1C2 lactadherin domain, which binds to phosphatidylserine on the outer membrane leaflet of some EVs. We investigated whether this approach could mediate EV targeting to T cells using an anti-CD2 scFv. We selected CD2 as a target for several reasons; ligand engagement triggers internalization of CD2, and we hypothesized that such a mechanism could enhance EV uptake upon receptor docking. This could be of particular utility for conferring delivery to T cells, which exhibit low rates of endocytosis and for which delivery of other vehicles is generally challenging. We also chose to avoid targets such as CD3 which could induce non-specific T cell activation. We selected a distinct display system based upon the platelet-derived growth factor receptor (PDGFR) transmembrane domain; we hypothesized that using this general strategy may confer display of targeting domains on multiple EV populations. Since extravesicular linker design may impact scFv trafficking, folding, and target binding, we investigated three candidates: an α-helix to provide structure, a 40 residue glycine-serine sequence to provide flexibility, or the hinge region of IgG4 used in chimeric antigen receptors to display scFvs on synthetic receptors. All three constructs were expressed at comparable levels in HEK293FT cells (FIGS. 7A-7B). To test display on EVs, two vesicle populations were isolated using a previously validated differential centrifugation method. EVs are best defined by the separation method used for their isolation; for convenience, hereafter the fraction isolated at 15,000×g is termed “microvesicles” (MV) and the fraction isolated at 120,416×g is termed “exosomes” (exo). Vesicles were enriched in canonical markers such as CD9, CD81, and Alix and depleted in the endoplasmic reticulum protein calnexin (FIG. 8A). Both populations comprised vesicles averaging ˜120-140 nm in diameter and exhibited the expected “cup shaped” morphology (FIGS. 8B-8C). Importantly, all three scFv display constructs were substantially expressed in both vesicle populations (FIG. 7C).

### Example 4: Display of Anti-CD2 scFvs Enhances EV Binding to Jurkat T Cells

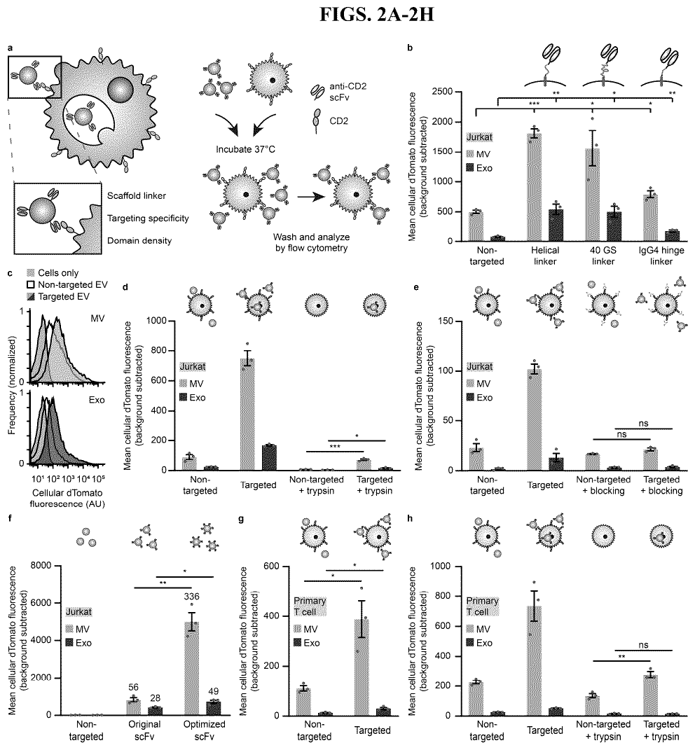

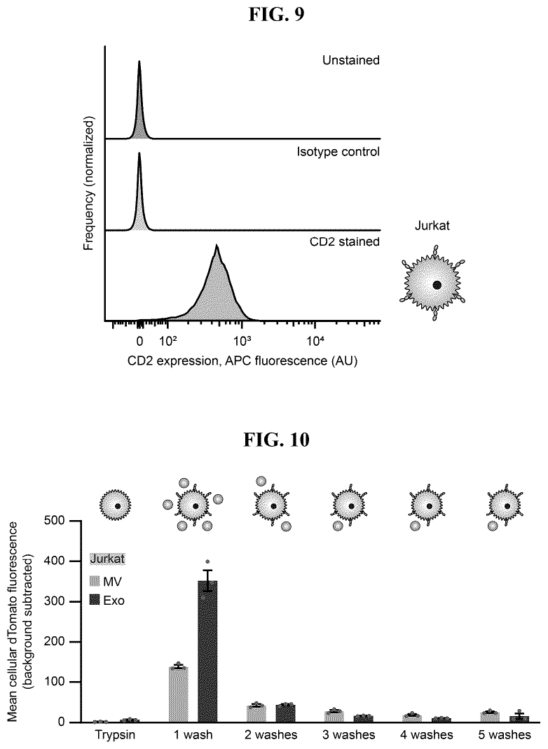

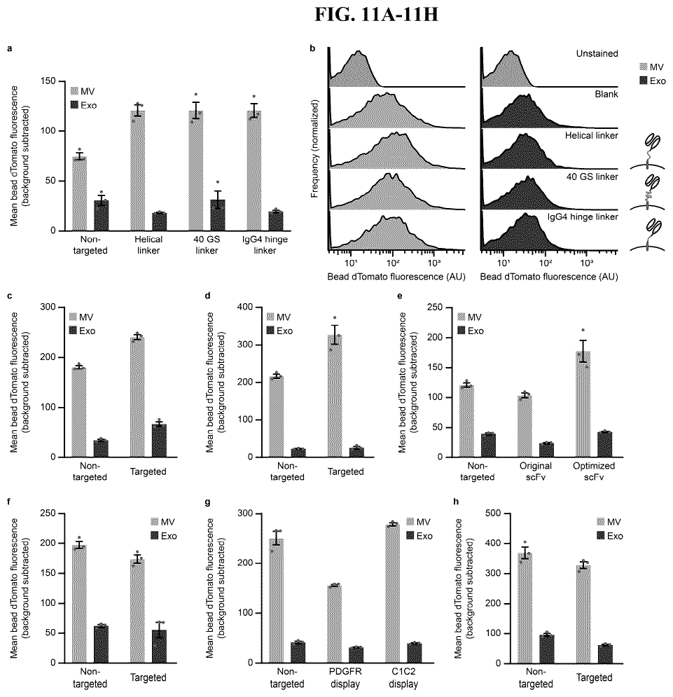

To evaluate targeting, we harvested vesicles from HEK293FT producer cells stably expressing our scFv constructs and a cytosolic dTomato fluorescent protein. EVs were incubated with Jurkat T cells, which express high levels of CD2 (FIG. 9), for 2 h, and then cells were washed to removed unbound vesicles (FIG. 10) and analyzed by flow cytometry. All three constructs enhanced both microvesicle and exosome binding to T cells (FIGS. 2A-2C). Display of scFvs on microvesicles enhanced delivery of dTomato to T cells more so than did display on exosomes, though some—but not all—of this effect is attributable to greater dTomato incorporation in microvesicles vs exosomes (FIGS. 11A-11B). Since the helical linker consistently conferred the greatest targeting effect, this design was carried forward for subsequent work.

### Example 5: CD2-scFv Binding Mediates Uptake by Recipient Cells

We next evaluated whether CD2 engagement by EVs triggers internalization (as noted, ligand binding naturally triggers CD2 internalization). To distinguish EV binding from uptake, cells were treated with trypsin after incubation with EVs to remove non-internalized vesicles. Cells receiving targeted vesicles displayed a modest increase in fluorescence over the non-targeted control (FIG. 2D), indicating that CD2 targeting can mediate EV uptake.

### Example 6: CD2-scFv-Mediated EV Targeting is Specific

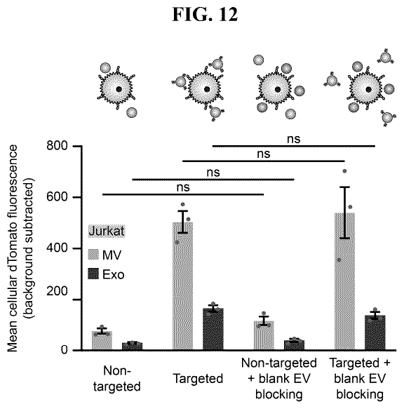

To determine whether the observed vesicle and T cell interactions resulted from specific receptor binding, we pre-incubated recipient Jurkat cells with an antibody binding the same T11 epitope on CD2 as does our scFv to block potential binding sites. Antibody pre-treatment ablated scFv-enhanced EV binding (FIG. 2E), demonstrating that our targeting is specific for CD2. In contrast, pre-incubation with non-targeted EVs (a potential non-specific competitor) did not substantially reduce either background or scFv-enhanced binding (FIG. 12). Together, these data indicate that the scFv mediates specific binding of EVs to CD2.

### Example 7: Optimization of scFv Expression Increases Targeting

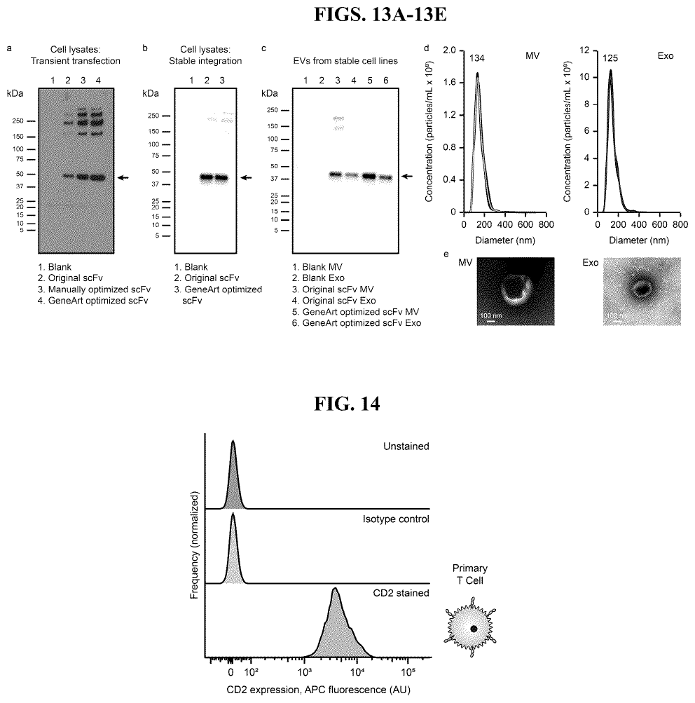

Increasing the avidity of interactions between binders (e.g., targeted therapeutics) and their receptors is a generally useful strategy for enhancing delivery and function in vivo.⁴³ To potentially capitalize upon this mechanism, we sought to increase the expression of our scFv constructs and therefore loading into vesicles through mass action. By optimizing the coding sequence of our scFv display construct for expression in human cells using a sliding window algorithm, we enhanced cellular expression of our scFv (FIGS. 13A-13B) and increased scFv loading onto vesicles without affecting vesicle size or morphology (FIGS. 13C-13E). EVs generated from cells stably expressing optimized scFv constructs exhibited enhanced specific binding to recipient cells (FIG. 2F). At the end of this limited optimization, targeted EV binding to CD2⁺ Jurkat T cells exceeded a 100-fold increase over non-targeted EVs. This optimized targeting system also conferred enhanced EV binding and modest EV internalization in primary human CD4⁺ T cells (FIGS. 2G-2H), which express high levels of CD2 (FIG. 14).

### Example 8: CD2-scFv Display Scaffold Influences Loading and Specificity

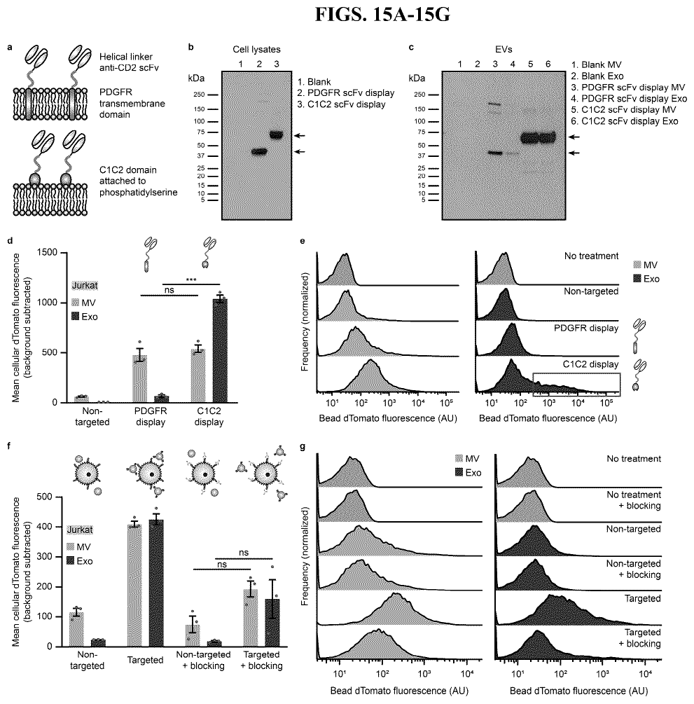

Previous reports have achieved scFv display on EVs by fusion to the C1C2 lactadherin domain, which binds to phosphatidylserine on the outer membrane leaflet of some vesicles. To compare our PDGFR-based display strategy to other state-of-the-art EV scFv display systems, we fused our optimized anti-CD2 scFv to the C1C2 lactadherin domain scaffold (FIG. 15A). We observed similar expression of both constructs in cells, but higher loading of C1C2 scFv constructs (as compared to PDGFR constructs) into vesicles (FIGS. 15B-15C). Both systems conferred similar microvesicle binding to Jurkat cells (FIG. 15D). C1C2 display appeared to confer some enhancement in exosome binding to T cells (compared to PDGFR display), but C1C2 display targeting was uneven, with only a subset of Jurkat recipient cells bound strongly to C1C2 display EVs, whereas PDGFR display targeting generally mediates delivery to the entire population of T cells (FIG. 15E). Since this pattern might provide evidence of CD2-independent EV binding (which would comprise an artifact), we investigated whether C1C2 display targeting was specific. Pre-incubation of EVs with an anti-CD2 antibody mediated only a partial reduction in C1C2 display targeted EV binding (in contrast to PDGFR display targeting), suggesting the existence of substantial non-target-specific mechanisms for C1C2 display targeting of EVs using this scFv (FIGS. 15F-15G). Given these observations, we opted to proceed with the validated and efficient PDGFR display of scFvs to achieve EV targeting.

### Example 9: Abscisic Acid-Inducible Dimerization Domains Enable an Active EV Cargo Loading System

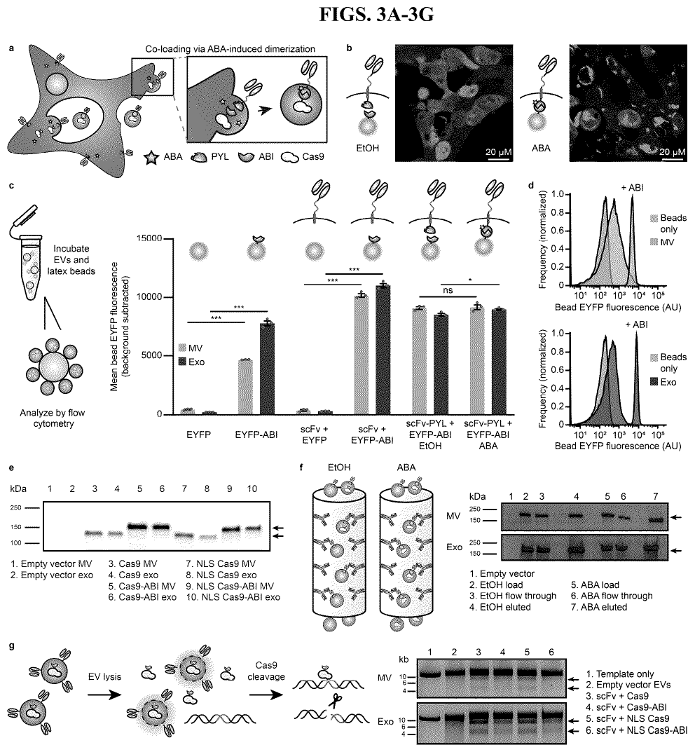

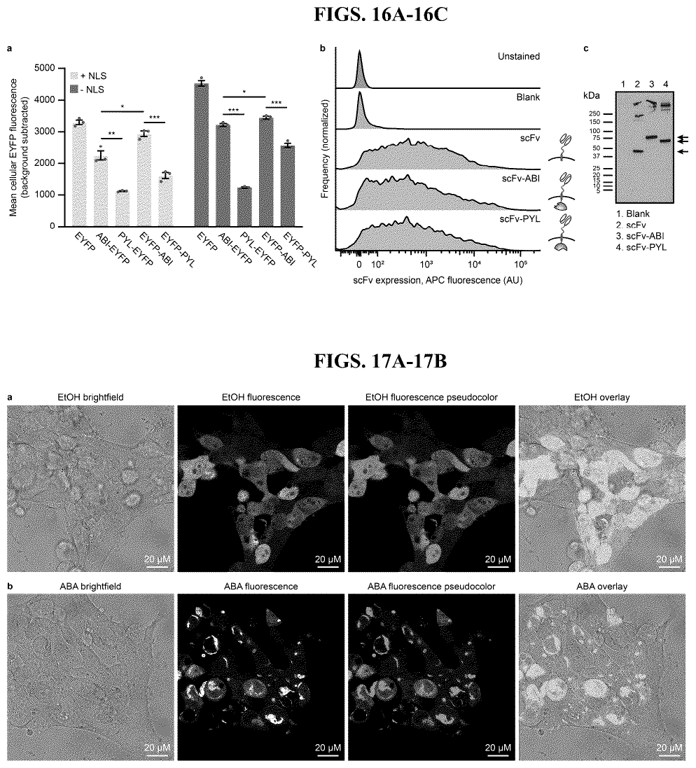

We next sought to engineer our scFv-containing EVs to load a therapeutic cargo of interest. Overexpression of cytosolic cargo in EV producer cells results in passive loading into vesicles during biogenesis via mass action. Increasing cargo content in EVs would potentially produce a more potent delivery vehicle. In order to both enhance cargo protein loading and increase the likelihood that a given vesicle will incorporate both a cytosolic cargo protein and our membrane-bound scFv, we designed a small entity-regulated dimerization-based loading system (FIG. 3A). Systems using light or small entities (e.g., rapamycin) as inducers have been reported to aid EV cargo loading, but light is difficult to scale to large volumes and rapamycin-induced dimerization is so tight that it is functionally irreversible. Therefore, we explored a new strategy based upon the plant hormone abscisic acid (ABA)-inducible interaction between truncated versions of the abscisic acid insensitive 1 (ABI) and pyrabactin resistance-like (PYL) proteins. This “ABA” system confers several advantages: association is rapid; the dimerization is reversible, presumably allowing for cargo release in recipient cells; ABA is inexpensive and non-toxic; and small molecule-regulated loading is more readily applicable to biomanufacturing than is control by light. We first investigated fusing the ABI and PYL domains to the luminal side of our scFv construct and to either the 5′ or 3′ end of a cytosolic or nuclear-localized EYFP cargo protein to determine effects on protein expression and function. Fusion with the PYL domain reduced expression (or destabilized) EYFP (FIG. 16A), while the scFv was tolerant to fusions with either ABI or PYL domains (FIGS. 16B-16C). Thus, we moved forward with the scFv-PYL and EYFP-ABI (3′ fusion) constructs. ABA-induced dimerization of ABI and PYL in this setup was readily evident by microscopy (FIG. 3B and FIGS. 17A-17B).

### Example 10: The ABI Domain Alone Drives Protein Incorporation into EVs

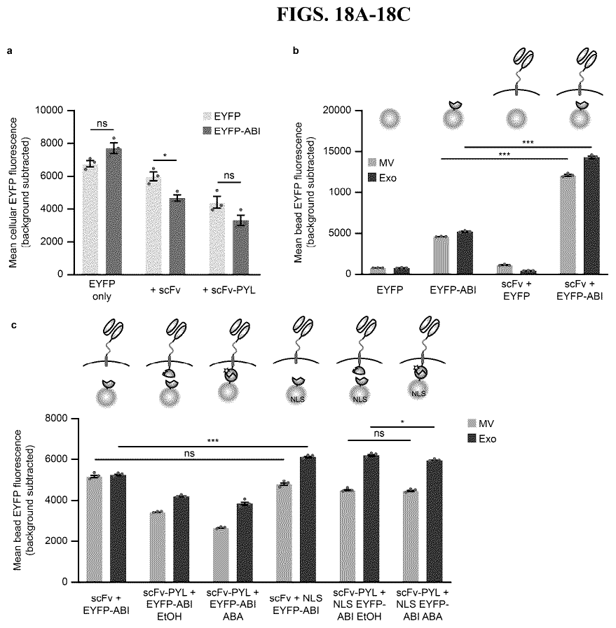

To investigate cargo protein loading, vesicles were adsorbed to latex beads and analyzed by flow cytometry. Surprisingly, no increase in EV loading was observed with ABA treatment, and across all conditions, constructs containing the ABI domain demonstrated a higher degree of loading than did those lacking this domain (FIGS. 3C-3D). This effect was not attributable to ABI-dependent increases of protein expression in producer cells (FIG. 18A). ABI-enhanced loading was evident when paired with the scFv alone or the scFv-PYL construct, indicating that intrinsic ABI-enhanced loading is independent of ABI-PYL interactions (FIG. 3C). The presence of the scFv conferred an added benefit in protein loading over an EYFP-ABI only control, for unknown reasons (FIG. 18B). In order to investigate the role of subcellular localization on the EV loading process, we introduced a nuclear localization sequence (NLS) to EYFP-ABI and compared loading to the purely cytosolic construct. ABA-induced dimerization again had a negligible effect on cargo loading, and addition of an NLS to EYFP-ABI did not diminish loading into EVs (FIG. 18C). Altogether, these data support the serendipitous discovery that ABI comprises a novel, potent EV cargo protein loading tag.

### Example 11: The ABI Domain Mediates Cas9 Loading into EVs

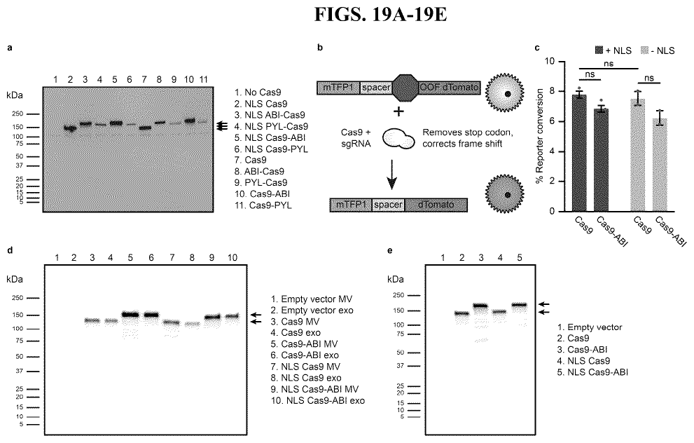

We next investigated whether ABI can be used to load EVs with functional cargo. As model, we selected S. pyogenes Cas9 ribonucleoprotein complexes (RNPs)—RNPs can be synthesized in producer cells (and are thus consistent with the GEMINI strategy) and because RNPs must travel to the nucleus of recipient cells to act on genomic targets, this system comprises a stringent test for functional EV-mediated delivery. ABI was fused to the N- or C-terminus of Cas9, and in general, expression patterns matched those observed for EYFP (FIG. 19A). Thus, we moved forward with the Cas9-ABI (3′ fusion) constructs. We also investigated whether addition of an NLS or ABI domain impacted Cas9 function. When expressed via transfection (along with a cognate sgRNA) in reporter Jurkat T cells, Cas9 fusion constructs exhibited similar nuclease activity (FIGS. 19B-19C). When Cas9 constructs were expressed in producer cells, the NLS minimally influenced Cas9 loading into EVs, while the ABI domain noticeably increased Cas9 loading (FIG. 3E and FIG. 19D) but not overall expression in producer cells (FIG. 19E). These trends are consistent with those observed with EYFP and demonstrate the utility of the ABI loading tag across multiple cargo proteins.

### Example 12: Membrane scFvs and ABI-Fused Cas9 Co-Load into EVs

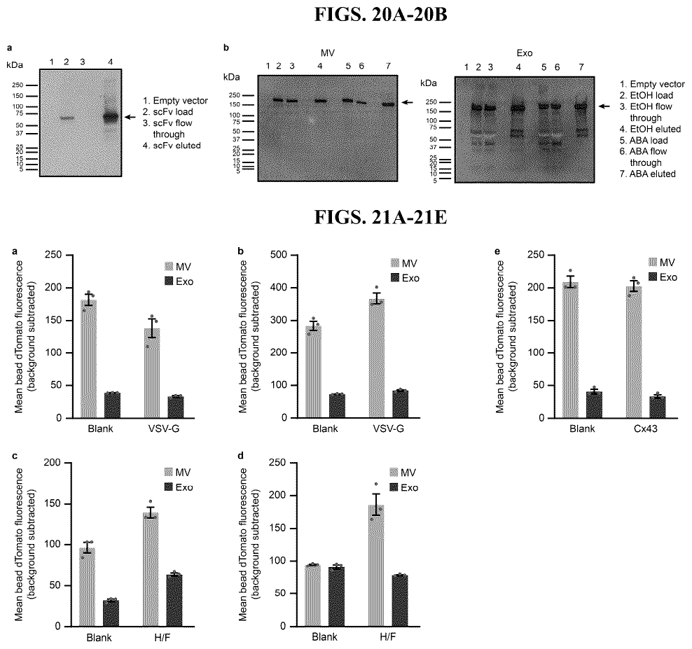

An important, but largely unexplored, factor to consider in engineering EV-based therapeutics is the extent to which multiple cargo types localize to the same vesicles in a population. Although ABI (alone) successfully loads protein into EVs, it remained unknown whether dimerization of cargo and display proteins could enhance co-loading into EVs (i.e., co-loading of both the scFv and Cas9 into individual vesicles). To evaluate this question, we generated vesicles from cells expressing scFv-PYL and Cas9-ABI treated with ABA or a vehicle control and isolated anti-CD2 scFv-displaying vesicles via the 3×FLAG tag located on the N-terminus of the scFvs by affinity chromatography (FIG. 20A). High levels of Cas9 were found in scFv-enriched vesicles, independent of ABA treatment, indicating that ABI-tagging of cargo is sufficient to achieve substantial scFv and Cas9 co-localization in EVs (FIG. 3F and FIG. 20B).

### Example 13: EV-Loaded Cas9 Exhibits Nuclease Function

To evaluate whether EV-encapsulated Cas9 RNPs are functional, we developed a direct in vitro assay. EVs from Cas9 and sgRNA-expressing cells were lysed and incubated with a plasmid encoding the sgRNA target sequence (FIG. 3G). Plasmids treated with lysed RNP-containing EVs showed the expected specific cleavage products under all conditions tested. The presence or absence of an NLS did not impact cleavage efficiency in this assay, but Cas9 fused to the ABI domain exhibited some reduced cleavage for both vesicle populations. This pattern contrasts with that observed in the transfection-based Cas9 assay (FIG. 19C), and thus it is not clear whether this partial effect (e.g., a potential reduction in Cas9 turnover rate) is meaningful in a cellular delivery context. Thus, both ABI+ and ABI− constructs were evaluated in subsequent Experiments.

### Example 14: Viral Glycoprotein Display Increases EV Uptake by T Cells

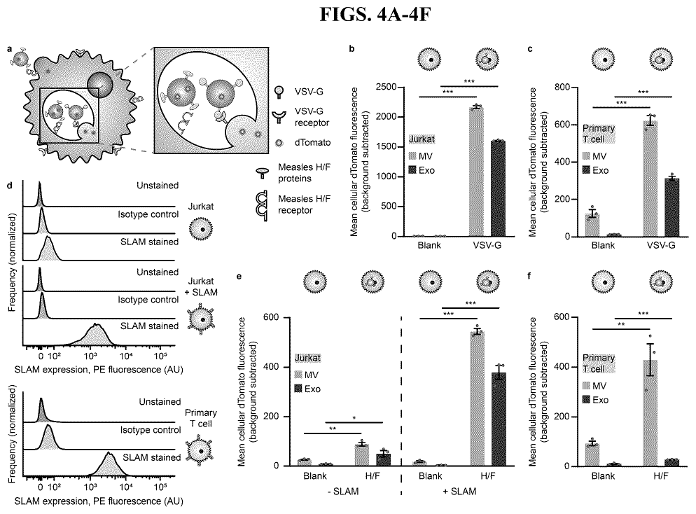

To promote EV uptake and fusion, we investigated displaying viral glycoproteins on EVs. We first investigated vesicular stomatitis glycoprotein (VSV-G), which is commonly used in lentiviral pseudotyping and has been reported to confer EV fusion with recipient cells. VSV-G was transiently expressed in dTomato-expressing producer cells, and the resulting EVs were incubated with recipient T cells for 16 h prior to trypsinization (to remove non-internalized vesicles) and analysis by flow cytometry. VSV-G enhanced EV uptake in both Jurkat T cells (FIGS. 4A-4B) and primary human CD4⁺ T cells (FIG. 4C), establishing the utility in of viral fusion proteins for delivering EVs to T cells.

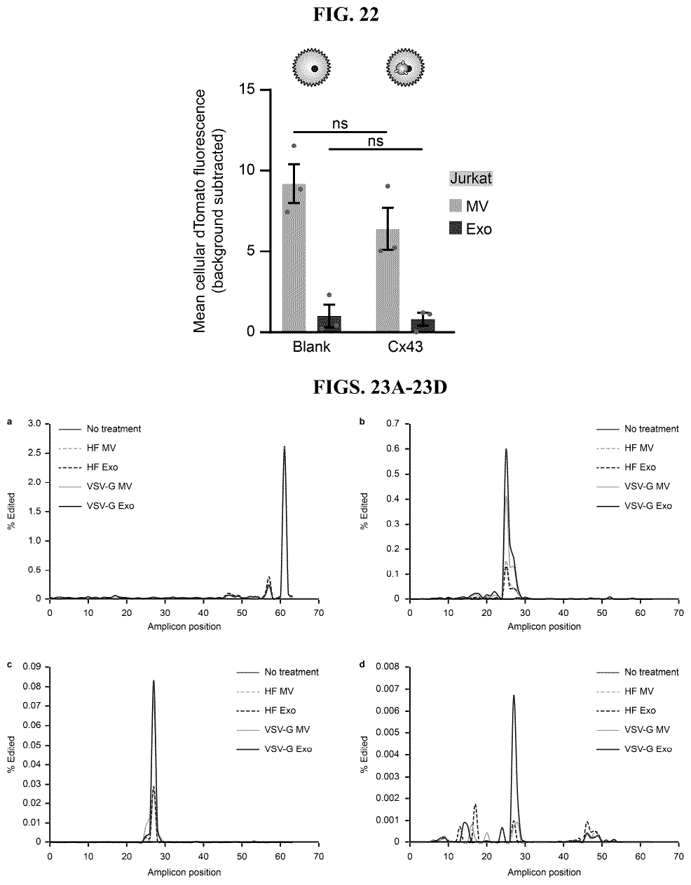

To develop an EV fusion system that is more specific to T cells (since VSV-G mediates fusion to most cell types), we investigated the use of truncated versions of the measles virus glycoproteins H and F which have previously been used to aid lentiviral delivery to T cells. These proteins bind signaling lymphocyte activation entity F1 (SLAM) and/or the complement regulator CD46, both of which are expressed on diverse T cells. H and F are classically believed to mediate viral fusion at the cell surface, although it has also been reported that viral endocytosis can be mediated by SLAM. In the same fluorescent EV uptake assay described above, we investigated EV delivery to Jurkats (which minimally express SLAM), SLAM-transgenic Jurkats, or primary human T cells that express SLAM (FIG. 4D). H and F proteins conferred modest EV uptake to parental Jurkats (SLAM-), but these proteins substantially enhanced EV uptake by SLAM-transgenic Jurkats and primary human CD4⁺ T cells (FIGS. 4E-4F). We also explored an alternative, non-viral protein-based strategy reported to promote functional transfer by overexpressing constitutively active Cx43, a connexin protein involved in the formation of gap junctions, on EV producer cells. Cx43 did not confer increased EV internalization by T cells in our hands, so this approach was not further investigated (FIG. 22). Altogether, these results support the use of the measles H/F glycoproteins as a method for enhancing EV uptake by SLAM+ T cells.

### Example 15: EVs Mediate Functional Delivery of Cas9 to Primary T Cells

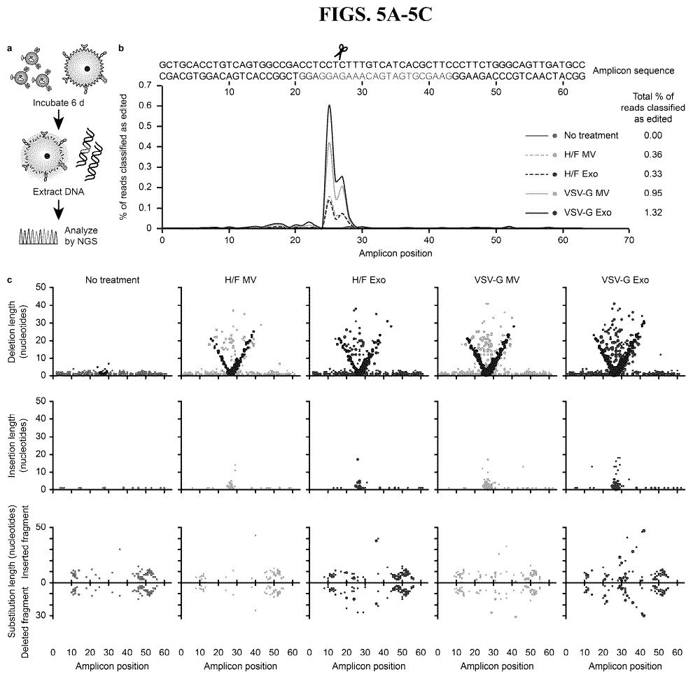

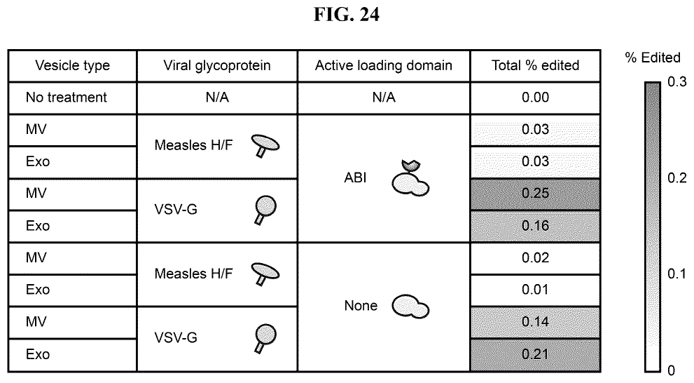

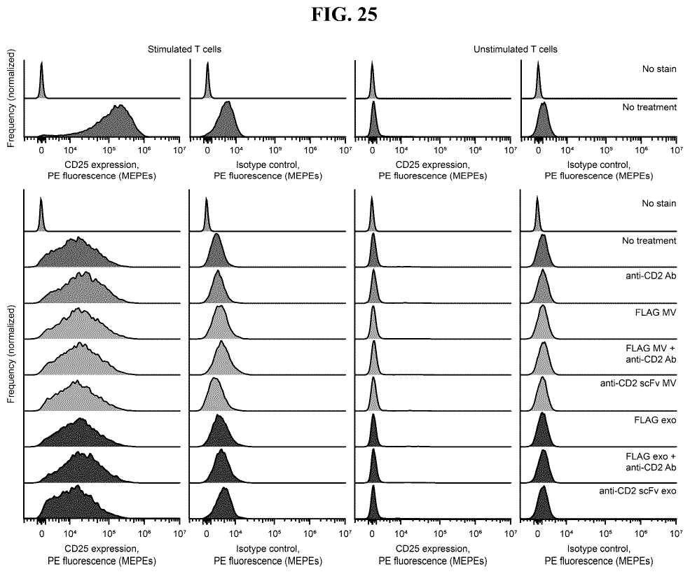

Evaluating functional delivery of Cas9 to recipient T cells requires effective cargo loading, T cell binding and fusion, and subsequent release of active Cas9 RNPs, and having validated each of these steps individually, we proceeded to evaluate the combined technologies—the first combined test of the GEMINI strategy. Specifically, we investigated the use of Cas9 to target the CXCR4 locus in primary T cells using a previously validated sgRNA. Since viral glycoprotein expression is cytotoxic, at this point we pivoted to biomanufacturing EVs using a Lenti-X HEK293T cell line that is well-suited to this challenge; this line was selected for its ability to produce high lentiviral titers. EVs containing the anti-CD2 scFv, NLS Cas9-ABI with the appropriate sgRNA, and either VSV-G or measles virus glycoproteins H/F were incubated with primary human CD4⁺ T cells for 6 d before harvesting genomic DNA for high throughput sequencing (HTS) to quantify and characterize targeted edits in a region of 64 nucleotides centered around the expected cleavage site. Excitingly, indels were identified at the predicted Cas9 cut site for all vesicle treatments containing Cas9 RNPs (FIGS. 5A-5C and FIGS. 23A-23D). VSV-G display on EVs conferred higher editing efficiencies than did measles H and F proteins, and exosome treatments conferred more edits than did microvesicle treatments for matched designs. The majority of edits were classified as deletions with a smaller number of insertion events or edits consisting of both an insertion and a deletion. This overall pattern is consistent with prior reports of Cas9 RNP editing at this locus, in that edits comprise mostly small deletions and insertions centered around the cleavage locus, indicating that EV-mediated delivery of Cas9 using GEMINI yields effects that are qualitatively comparable to electroporation of recombinant Cas9 RNPs. In order to evaluate the role of ABI-mediated active loading in functional delivery, we generated EVs with Cas9+/− ABI and evaluated editing efficiencies in primary T cells. The two Cas9 variants performed comparably well in this context, despite previously noted tradeoffs in loading and specific cleavage activity (FIG. 24).

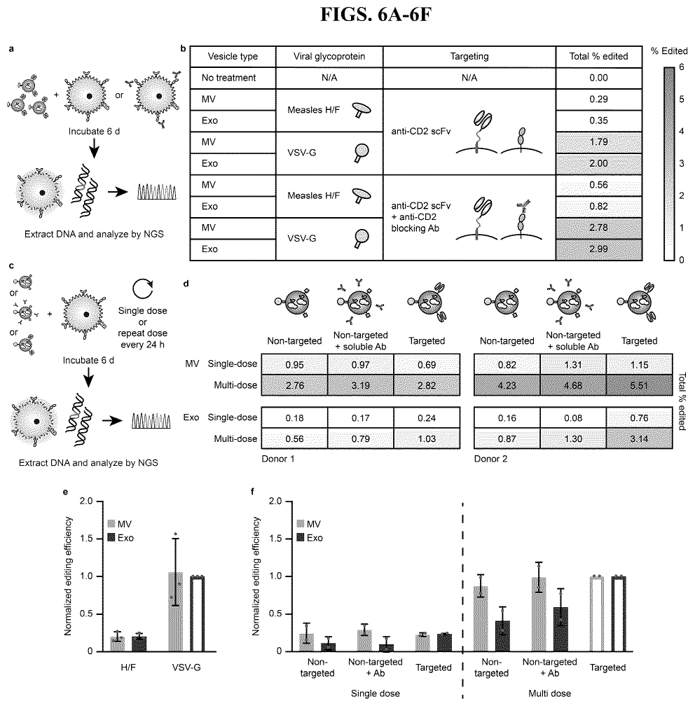

Having achieved functional delivery with our multifunctional EVs, this enabled us to next interrogate the specific contributions of each engineered EV feature. In particular, we sought to evaluate the unique contribution of the anti-CD2 scFv, since it can confer some degree of binding and uptake in vitro. To ascertain the requirement of EV scFv-CD2 engagement for functional delivery, we pretreated cells with an anti-CD2 antibody prior to EV addition to block receptors on recipient cells. Surprisingly, we found that pretreatment with the anti-CD2 antibody increased editing rates across vesicle types and viral glycoprotein systems (FIGS. 6A-6B). To explain this observation, we hypothesized that engagement of CD2 might result in higher levels of T cell activation, making cells more susceptible to EV uptake and editing; this would be a novel consequence of CD2 engagement if confirmed. To investigate this possibility, we incubated T cells with either EV scFvs or anti-CD2 antibodies and analyzed surface expression of CD25 2 d post-treatment. CD25 expression was minimally impacted by any treatment, indicating that T cell activation cannot explain the observed increase in editing upon CD2 engagement (FIG. 25). To investigate how editing efficiency scales with practical considerations such as EV dose, and to probe how CD2 engagement may contribute to this process, we evaluated EV delivery to T cells from two distinct donors using only a single EV dose or repeating EV administration every day for the 6 d incubation (FIGS. 6C-6D). As expected, repeat EV administration increased editing efficiency in all cases, indicating that redosing is a useful handle for boosting editing. In general, scFv-CD2 engagement enhanced editing mediated by exosomes, although this effect was not evident for microvesicle-mediated editing. Finally, in order to evaluate which trends hold across experiments, we performed a combined analysis (normalizing to control for variables hypothesized to contribute to variation in editing efficiency, such as donor T cell batch-specific susceptibility to Cas9 RNP-mediated editing) (FIGS. 6E-6F). Overall, these combined trends support the key conclusions noted above.

### Example 16: Discussions

In this study, we developed a strategy of combining genetically-encoded, general platform approaches for targeting EVs to recipient cells with surface-displayed scFvs, actively loading EVs with protein cargo via tagging with vesicle-localizing domains, and promoting uptake and fusion with recipient cells by displaying viral glycoproteins. The motivating application of achieving Cas9 delivery to T cells-a challenging objective-proved useful for refining and validating technologies that can be combined to achieve this goal.

An exciting aspect of EV-mediated delivery is the potential to target vesicles to cells and receptors of interest through engineered interactions. Prior reports have demonstrated non-targeted EV-mediated transfer to T cells, with cargo including EV-encapsulated AAV8 or zinc finger-fused methyltransferases. EVs that bind T cells have also been described as a method of crosslinking T cells and other cellular targets by displaying linked anti-CD3 and anti-EGFR scFvs on the PDGFR transmembrane domain. To our knowledge, this study is the first demonstrating integration of EV targeting and uptake by T cells. We anticipate that the modularity of our targeting construct will be useful for directing EVs to other cell types and receptors.

The key technology reported here involved the serendipitous discovery that the ABI domain (from the ABA dimerization system) facilitates EV cytosolic cargo protein loading even without with ABA. The mechanism of this effect is unknown. ABI is not predicted by WoLF PSORT (genscript.com/wolf-psort.html) to localize to the cell membrane or endosomal pathways. An advantage of this system is that ABI-mediated loading is easier to implement than multi-domain dimerization systems (using light, rapamycin, or Dmr domains) or tags that require overexpression of helper proteins to facilitate trafficking into vesicles, such as the WW domain and Ndfip1. Other active loading tags have recently been explored by Codiak Biosciences, in this case deriving a tag from a membrane-associating protein, though the reversibility of such interactions has yet to be established. Although increased Cas9 loading did not confer additional DNA cleavage in our in vitro assay, potentially because this particular Cas9 fusion strategy reduced Cas9 turnover rate (FIG. 19C), higher cargo loading is likely beneficial in cell delivery contexts where EVs must overcome additional barriers of uptake, fusion, cytosolic release, and intracellular trafficking. In such contexts, the advantage of a higher dose with more shots on goal may outweigh slower reaction rates. It is also possible that the ABI fusion strategy may be refined in future work to mitigate any effects on Cas9 activity.

The eventual fate of EVs in recipient cells is often degradation in the endosomal/lysosomal pathway, and thus developing methods to achieve vesicle fusion in recipient cells is desirable for achieving (or enhancing) functional cargo delivery (i.e., to the cytoplasm). Here, we demonstrated the use of VSV-G and measles virus glycoproteins H/F to achieve efficient internalization of EVs by both Jurkat and primary T cells for VSV-G and in cells expressing the lymphocyte receptor SLAM for H/F. An important translational consideration is that mutant versions of the H/F proteins have been developed to evade neutralizing host antibodies, such as those induced by the measles vaccine. However, in functional Cas9 delivery studies, we observed greater Cas9 editing efficiencies in primary T cells treated with VSV-G vesicles as compared to H/F, likely because of increased fusion of VSV-G in acidic endosomal environments. Our observed conversion efficiencies, although modest at the doses used in this exploration, meet or exceed comparable reports in the literature. Perhaps the most rigorous and compelling comparator study reported that 12 repeat, high-dose (˜1×10¹¹ EVs as compared to our ˜1×10¹⁰ EVs) administrations of vesicles derived from MDA-MB-231 breast cancer cells loaded with an sgRNA were required to achieve conversion efficiencies on the order of 0.1% in HEK293T reporter cells that constitutively express Cas9 (a cell type to which delivery of viral vectors and various biomolecules is fairly efficient compared to T cells). We observed substantially greater conversion rates in our system, and conversion increased with repeat administration for both EV types. In the specific HIV application contemplated, conversion of even a limited pool of T cells to resist HIV infection could confer therapeutic benefits. EVs have been explored for potential utility in HIV treatment through approaches such as Cas9-mediated excision of proviruses in microglial cells, repressing viral replication with zinc finger-fused methyltransferases, or killing of infected cells using HIV Env-targeted vesicles, but these preliminary demonstrations have not yet been developed into methods for achieving specific delivery and treatment of T cells using a clinically translatable approach. Another important finding is that while exact editing efficiencies varied across donors and EV doses (a pattern observed with Cas9 RNP delivery by other methods), the overall trends were highly conserved when controlling for these effects, demonstrating repeatability. These results are particularly exciting when noting that the quantified efficiencies are limited by Cas9-mediated cleavage and DNA repair rates, such that we are certainly underestimating the number of functional delivery events, and other cargo types and mechanisms might confer even greater rates of functional delivery.

A surprising finding is that CD2 engagement, by either recombinant antibody or EV-displayed antibody, enhanced functional exosome-mediated delivery in vitro, though no comparable benefit for microvesicle-mediated delivery was observed. This combination of effects is not explained by known features of CD2/T cell biology, although it could be related to findings that ligand engagement triggers CD2 internalization. While scFv-displaying vesicles of both types specifically bound CD2 and were internalized to some degree, there may exist a difference in intracellular trafficking and fusion between the two vesicle populations. For example, CD2-binding-mediated trafficking might favor fusion over native uptake pathways in a way that differentially favors exosomes. This phenomenon warrants further study to elucidate underlying mechanisms.

A notable feature of this study was the selection of methods that avoid artifacts found in EV studies. One general and often overlooked artifact with EV functional delivery experiments is the risk of transfer of residual producer cell transfection reagent; particles from cells transfected with lipoplexes can mediate functional effects erroneously attributed to EVs. We minimized such risks by employing a transfection method that is unlikely to transfer plasmids to T cells. Key comparative observations (e.g., differences in functional delivery by viral glycoprotein choice) support the interpretation that true EV-mediated delivery was quantified.

### Example 17: Prophetic Functional Delivery of Cas9 to T Cells

A further contemplated example includes using our active loading strategy to deliver Cas9-sgRNA complexes to recipient cells to mediate gene editing. An example of this strategy would be to express Cas9-ABI (fused using our technology in one of the implementations contemplated here) and an anti-CXCR4-targeting sgRNA in HEK293FT cells; to harvest the EVs produced from these cells using standard methods; to deliver these EVs to T cells (e.g., Jurkat T cells or primary human T cells); and after some time, to evaluate whether the CXCR4 locus has been cut and repaired in these recipient T cells (e.g., using high throughput sequencing).

The technologies employed here are generalizable and amenable to large scale production and biomanufacturing. Our strategy of genetically programming the self-assembly of multifunctional particles avoids the need for post-harvest chemical modification that necessitates further purification, lower EV yields, and may incur regulatory challenges. Although we used transient transfections for some transgenes (e.g., viral glycoproteins that cannot be constitutively expressed due to toxicity), such genes are regularly expressed from inducible promoters for production of biologics. We anticipate that the integrated tools developed here for cell-derived membrane particle (CDMP) cargo loading, and vesicle fusion will be widely applicable for a range of applications and targets, providing a flexible platform for engineering CDMPs therapeutics.

### Example 18: Functional Delivery to Jurkat T Cells

The present Example demonstrates the ability of extracellular vesicles comprising a lentivirus core (EV-LV), a T cell targeting domain displayed on a PDGFR transmembrane, and a fusogen to fuse with Jurkat T cells.

EV-LV were designed to display
1) no targeting domain combined with no VSVG, VSVGwt, or VSVGmut;
2) Anti-CD2 targeting domain combined with no VSVG, VSVGwt, or VSVGmut; or
3) Anti-CD5 targeting domain combined with no VSVG, VSVGwt, or VSVGmut.

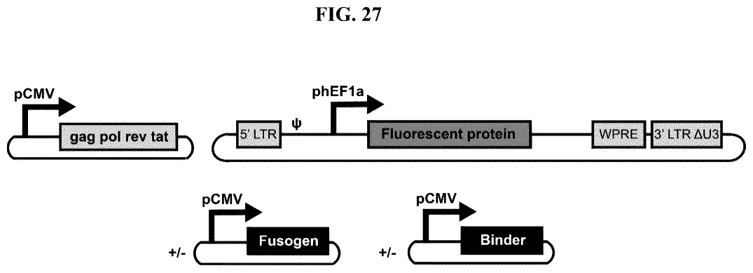

The production cell line LentiX used in this Example to produce EV-LV is a subclone of the transformed embryonic kidney cell line, HEK293. LentiX are a lentivirus producing cells and are engineered to stably express SV40 large T antigen. LentiX were plated and transfected with one or more plasmids (via calcium-phosphate precipitation) depending on the above study design. FIG. 27 shows plasmid constructs used in Example 18. Cell media was changed appropriately. The produced lentiviruses were harvested and physical titer was determined by qPCR.

Jurkat T cells or HEK293FT cells were next transduced with 5 doses of unconcentrated (logarithmically spaced) virus, with or without 8 μg/ml polybrene (a polymer which reduces electrostatic repulsion between viral and recipient cell membranes and may improve transduction). Flow-based fluorescent readout was performed two days after transduction and qPCR for particle titer in parallel.

EV-LV with VSV-Gwt Delivery to Jurkat T Cells.

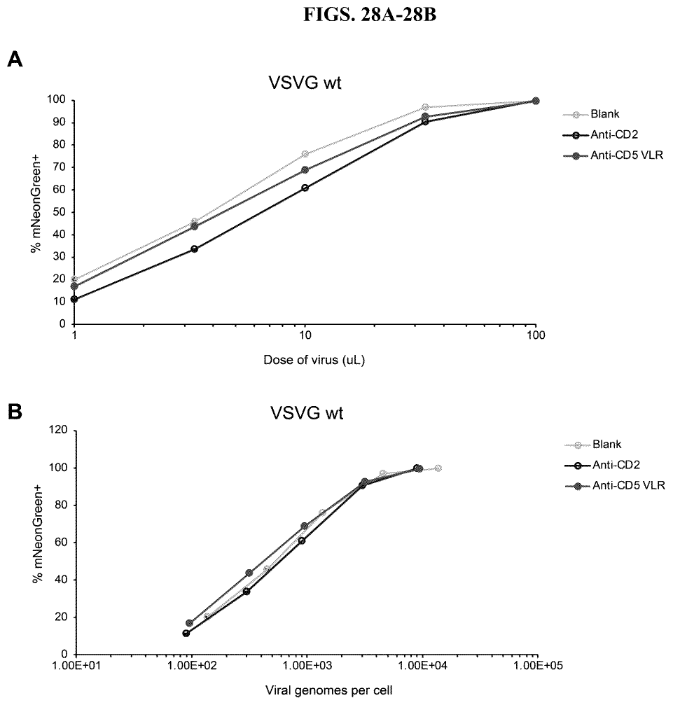

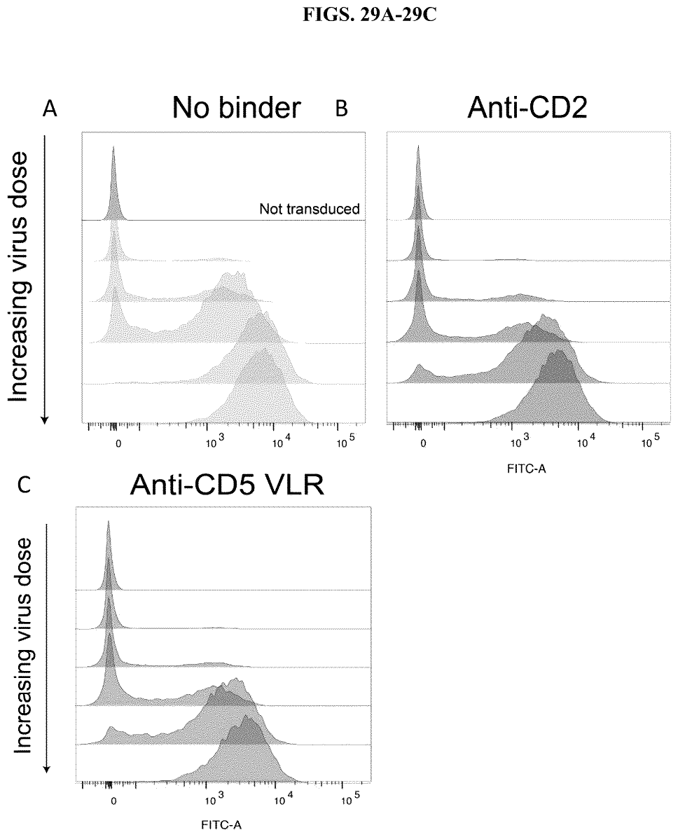

All EV-LV displaying VSV-Gwt provided good transduction of Jurkat T cells. A dose of 30-100 ul of EV-LV displaying VSV-Gwt transduced 80-100% of Jurkat T cells (FIG. 28A). Viral genomes per cell derived from qPCR titer results and 40,000 cells plated per well are shown in FIG. 28B. FIGS. 29A-29C depict the population-level histograms obtained by flow cytometry for representative samples of each of the conditions reported in FIGS. 28A-28B.

EV-LV with VSV-Gmut Delivery to Jurkat T Cells.

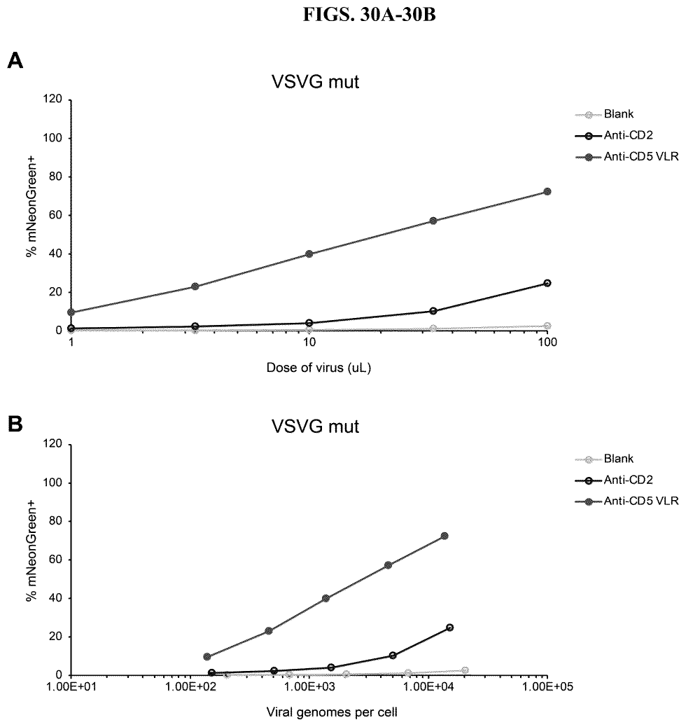

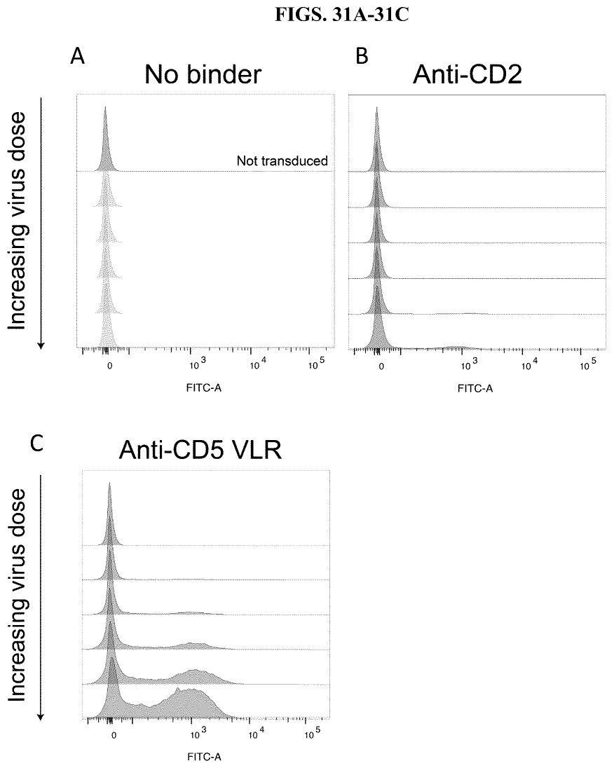

EV-LV displaying VSV-Gmut (alone) minimally transduce Jurkat T cells (up to 3.5%). Co-expression of VSV-Gmut with one of the targeting domains (anti-CD2 or anti-CD5) rescues infectivity with VSV-Gmut alone. (FIG. 30A). Viral genomes per cell derived from qPCR titer results and 40,000 cells plated per well (FIG. 30B). FIGS. 31A-31C depict the population-level histograms obtained by flow cytometry for representative samples of each of the conditions reported in FIGS. 30A-30B.

EV-LV without any VSV-G Delivery to Jurkat T Cells.

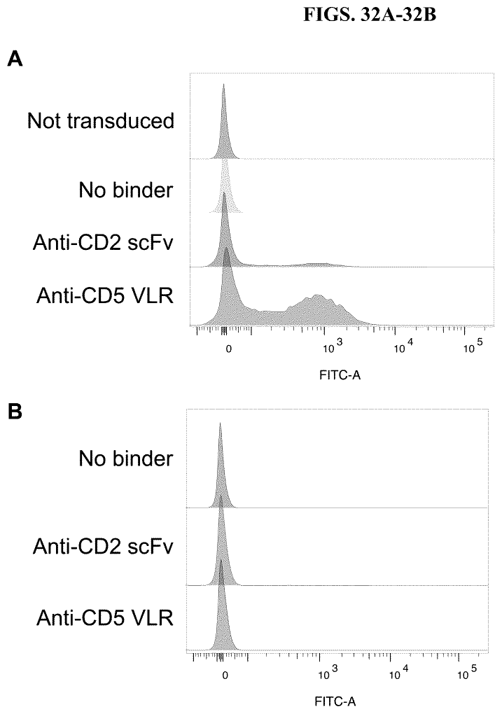

Viruses without any VSV-G (e.g., VSV-Gwt or VSV-Gmut) generally do not infect Jurkats. This may be due to protein transfer and not transduction based on the low magnitude of fluorescent signal (FIGS. 32A-32B).

EV-LV with Various VSV-G Delivery to HEK293FT Cells.

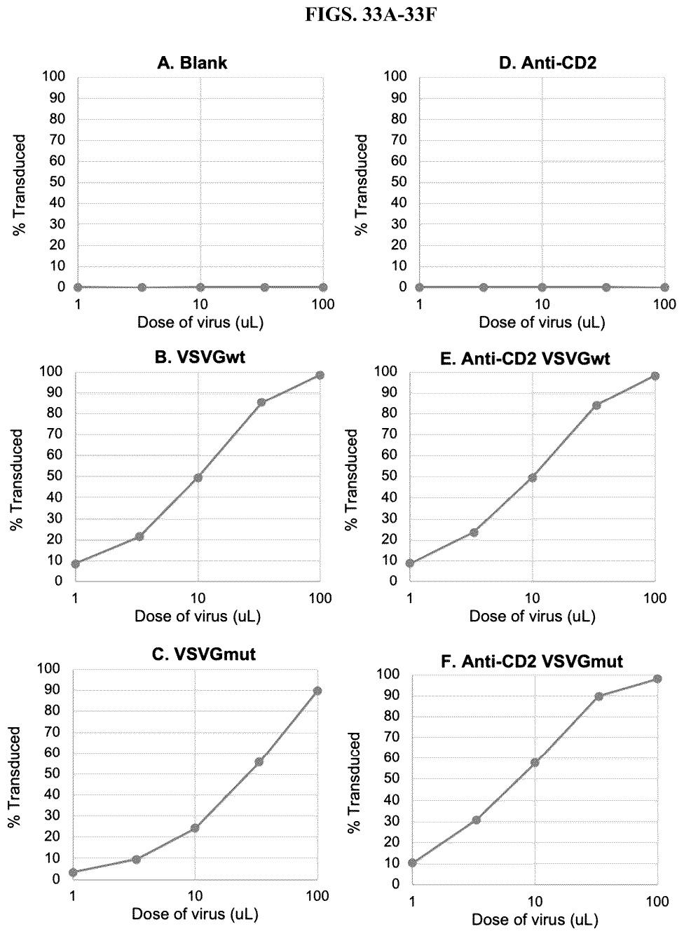

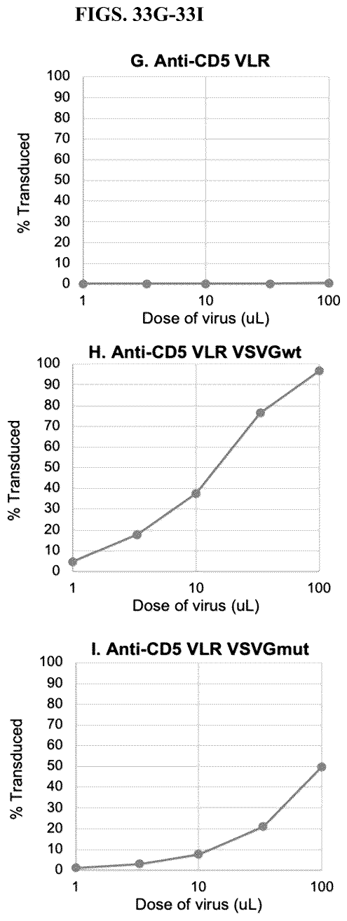

Viruses without VSV-Gwt or VSV-Gmut do not infect HEKs (FIG. 33A, 33D, 33G). Viruses with VSV-Gmut (FIG. 33C, 33F) infect similarly to those with VSV-Gwt (FIG. 33B, 33E, 33H). Except anti-CD5 VLR which transduces less with VSV-Gmut (FIG. 33I).

EV-LV particles displaying one of two T-cell targeting domains (anti-CD5 VLR or anti-CD2 scFv) and displaying binding-incompetent VSV-Gmut transduced Jurkat T cells in a dose-dependent manner.

### Example 19: Functional Delivery to Primary Human T Cells

The present Example demonstrates the ability of extracellular vesicles comprising a lentivirus core (EV-LV), a T cell targeting domain displayed on a PDGFR transmembrane domain, and a fusogen to fuse with primary human T cells (CD4+/CD8+).

The following T cell targeting domains and a fusogen combination were tested:
No surface modification ver1
No surface modification ver7
VSV-Gwt
VSV-Gmut
anti-CD5 VLR
VSV-Gmut+anti-CD5 VLR
VSV-Gwt+anti-CD5 VLR ver1
VSV-Gwt+anti-CD5 VLR ver7

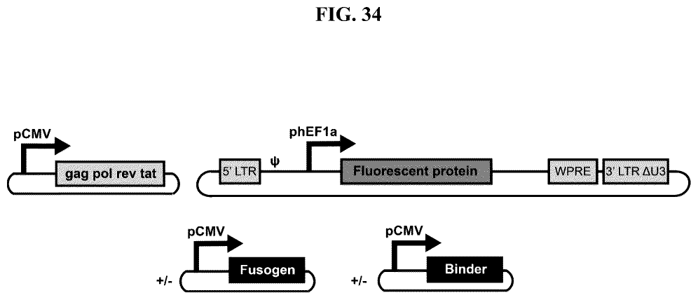

LentiX (lentivirus producing cells) were plated and transfected with one or more plasmids (via calcium-phosphate precipitation) depending on the above study design. FIG. 34 shows the plasmid constructs used in Example 19. Cell media was change appropriately. The produced EV-LVs were harvested. Physical titer was determined by qPCR. Equal genome copies of virus to T cells and HEK29FT cells were applied. Flow-based fluorescent readout was performed three days after transduction and concurrent staining to identity T cell identity as CD4⁺ and/or CD8⁺ was performed.

EV-LV with VSV-Gmut or VSV-Gmut Delivery to Primary Human T Cells

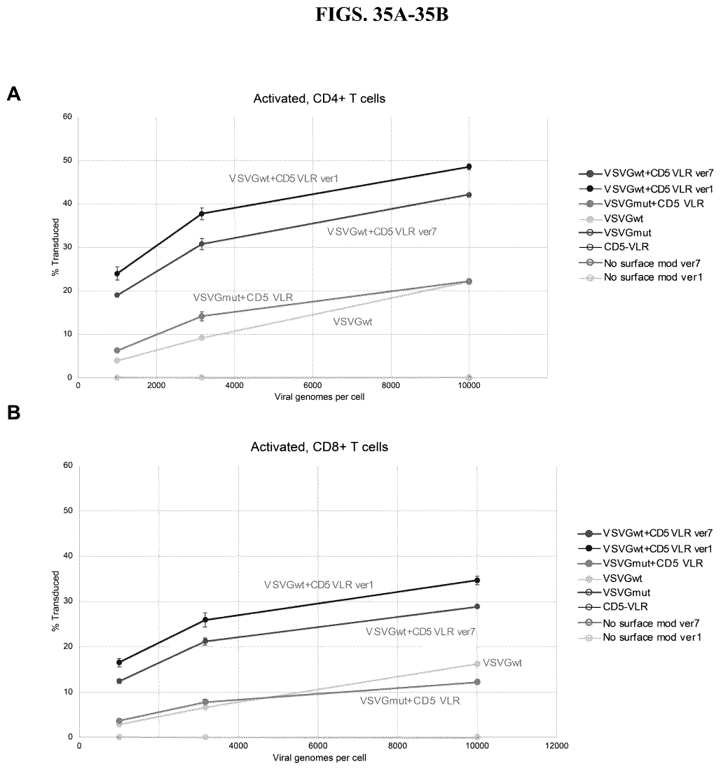

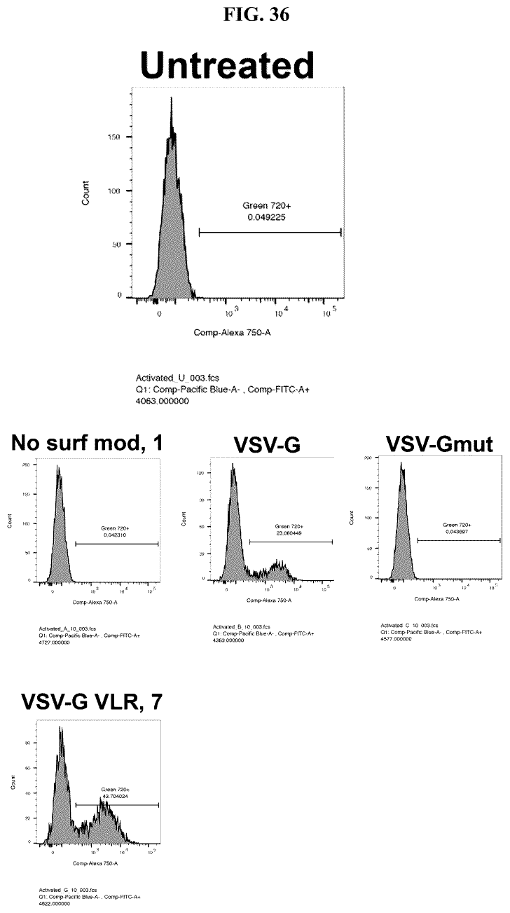

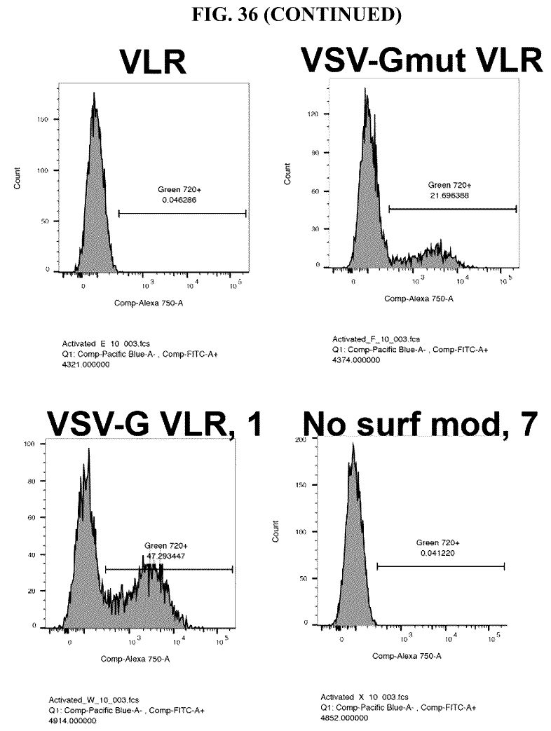

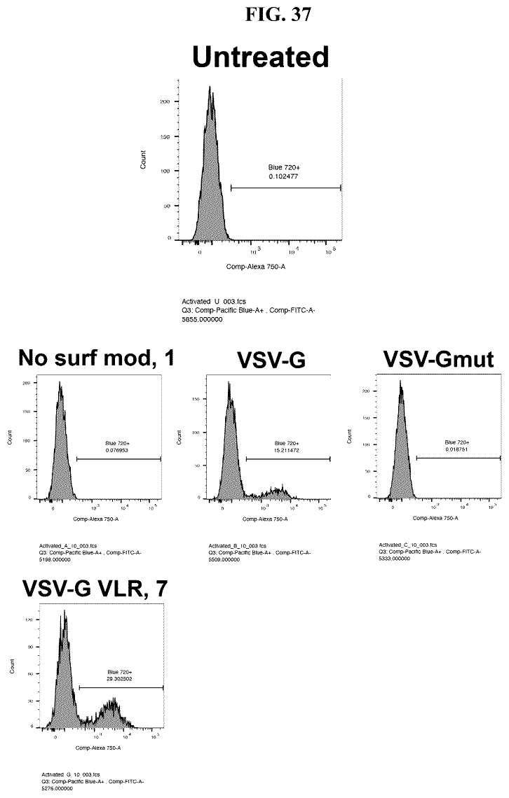

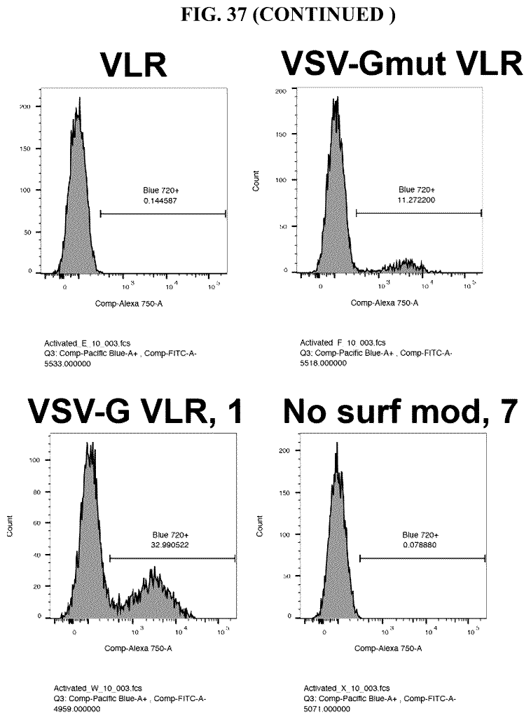

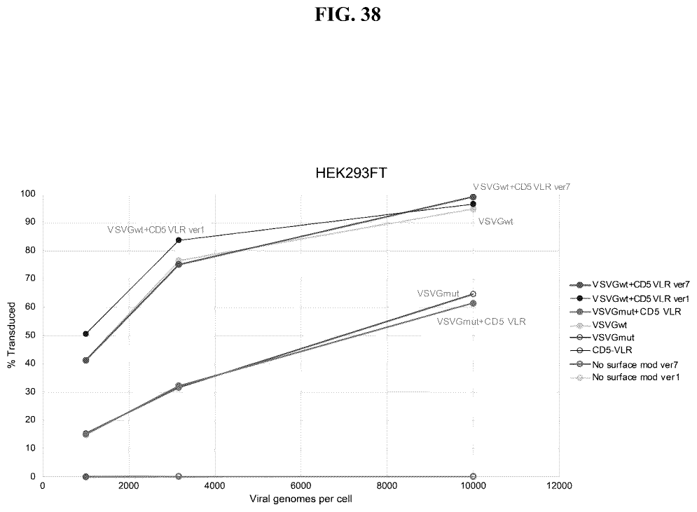

All EV-LV displaying VSV-Gwt yielded dose-dependent transduction of activated T cells (FIGS. 35A and 35B). Though no transduction was observed with VSV-Gmut alone. EV-LV displaying anti-CD5 VLR and VSV-Gmut demonstrated transduction of activated T cells as good as or better than VSV-Gwt (FIGS. 35A and 35B).

EV-LV with VSV-Gmut or VSV-Gmut Delivery to HEK293TF Cells.

All EV-LV displaying VSV-G (wt and mut) yielded dose-dependent transduction of HEK293FT cells. Addition of anti-CD5 VLR with VSV-Gwt does not result in substantially higher transduction than VSV-Gwt alone. Addition of anti-CD5 VLR with VSV-Gmut does not result in higher transduction than VSV-Gmut alone. Both of these observations are likely attributed to the fact that HEK293FTs do not express CD5.

EV-LV particles displaying anti-CD5 VLR and binding-incompetent VSV-Gmut transduce primary T cells in a dose-dependent manner. Inclusion of anti-CD5 VLR with VSV-Gmut restores infectivity of particles to be as efficient at transducing activated T cells compared to VSV-Gwt alone. EV-LV particles displaying anti-CD5 VLR and VSV-Gwt transduce primary T cells ˜5× better than gold standard EV-LV particles displaying VSV-Gwt alone.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

What is claimed is:

1. A population of engineered lipid bilayer particles comprising:
(i) a targeting chimeric polypeptide comprising:
(a) a targeting domain arranged so that the targeting domain is on the particle surface, wherein the targeting domain comprises a binding moiety that specifically binds to a target ligand on surfaces of recipient cells of interest;
linked directly or indirectly with
(b) a transmembrane domain;
and
(ii) a fusogen entity polypeptide comprising:
(a) fusogen moiety arranged so that the fusogen moiety is on the particle surface;
linked directly or indirectly with
(b) a transmembrane domain.

2. The population of claim 1, wherein the targeting domain is or comprises an antibody agent.

3. The population of claim 2, wherein the antibody agent is a single chain antibody agent.

4. The population of claim 2, wherein the antibody agent is selected from the group consisting of an antibody, a Fab, a Fab′, a F(ab′)₂, a Fd, a scFv, a single-chain antibody, a disulfide-linked Fvs (sdFv), an affinibody, a DARPIN, a nanobody, a variable lymphocyte receptor (VLR), and a camelid antibody.

5. The population of claim 1, wherein the targeting domain is or comprises a DARPIN or other engineered high affinity binding polypeptides.

6. The population of claim 1, wherein the targeting domain is a scFv.

7. The population of claim 1, wherein the transmembrane domain of targeting chimeric polypeptide comprises AVGQDTQEVIVVPHSLPFKVVVISAfLALVVLTIISLIILIMLWQKKPR (SEQ ID NO: 18), a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to SEQ ID NO: 18, or a functional fragment thereof.

8. The population of claims 1 to 7, wherein the targeting domain comprises an amino acid sequence of
(SEQ ID NO: 20)
NIMMTQSPSSLAVSAGEKVTMTCKSSQSVLYSSNQKNYLAWYQQKPGQSP
KLLIYWASTRESGVPDRFTGSGSGTDFTLTISSVQPEDLAVYYCHQYLSS
HTFGGGTKLEIKRGGGGSGGGGSGGGGSQLQQPGAELVRPGSSVKLSCKA
SGYTFTRYWIHWVKQRPIQGLEWIGNIDPSDSETHYNQKFKDKATLTVDK
SSGTAYMQLSSLTSEDSAVYYCATEDLYYAMEYWGQGTSVTVSS.

9. The population of claims 1 to 8, further comprising a first cargo entity connected to the transmembrane domain of the targeting chimeric polypeptide via a linker.

10. The population of claim 9, wherein the linker comprises:
(1) an amino acid sequence selected from SEQ ID NO: 10 (TSGGGGSGGGSGGGS), SEQ ID NO: 12 (TRGGGGSGGGSGGGS), SEQ ID NO: 14 (GGGGSGGGSGGGSTG), SEQ ID NO: 15 (DQSNSEEAKKEEAKKEEAKKSNS), SEQ ID NO: 16 (SGGGSGGGSGGGSGGSGGSGGGSGGSGGSGGGSGGGSGGG), and SEQ ID NO: 17 (ESKYGPPAPPAP); or
(2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 10, 12, 14, 15, 16, or 17.

11. The population of claims 1-10, wherein the engineered lipid bilayer particles are cell-derived membrane particles (CDMPs).

12. The population of claim 11, wherein the CDMPs are selected from extracellular vesicles, virus particles, virus-like particles (VLPs), apoptotic bodies, platelet-like particles, and combinations thereof.

13. The population of claim 12, wherein the CDMPs are extracellular vesicles selected from the group consisting of exosomes, microvesicles, and combinations thereof.

14. The population of claims 1 to 13, wherein the fusogen entity polypeptide is a viral fusogen.

15. The population of claims 1 to 13, wherein the fusogen entity polypeptide is a non-viral fusogen.

16. The population of claim 14, wherein the viral fusogen is a polypeptide from vesicular stomatitis virus, Measles virus, Sindbis virus, Tupaia paramyxovirus, Nipah virus, Chandipura virus, Rabies virus, Lymphocytic choriomeningitis virus, Mokola virus, Ross River virus, Ross River virus, Semliki Forest virus, Venezuelan equine encephalitis virus, Ebola virus, Marburg virus, Lassa virus, Avian leukosis virus, Jaagsiekte sheep retrovirus, Moloney Murine leukemia virus, Gibbon ape leukemia virus, Feline endogenous retrovirus (RD114), Human T-lymphotropic virus 1, Human foamy virus, Maedi-visna virus, SARS-CoV, SARS-CoV-2, Sendai virus, Respiratory syncytia virus, Human parainfluenza virus type 3, Human parainfluenza virus type 4, Hepatitis C virus, Hepatitis C virus, Influenza virus, Fowl plague virus, Autographa californica multiple nucleopolyhedro virus, Baboon endogenous retrovirus, Cocal virus, Japanese encephalitis virus, Dengue virus, Zika virus, West Nile virus, Yellow fever virus, Tick-borne encephalitis virus, Herpes simplex virus 1, Hendra virus, Newcastle disease virus, Epstein Barr virus, Bourbon virus, Varicella-zoster virus, Severe fever with thrombocytopenia virus, Hantavirus, Vaccinia virus, Simian immunodeficiency virus, Human immunodeficiency virus, Junin virus, Machupo virus, Bas-Congo virus, La Crosse virus, Human cytomegalovirus, Human cytomegalovirus, Thogoto virus, or Dhori virus.

17. The population of claim 14, wherein the viral fusogen is selected from a lentiviral glycoprotein or a glycoprotein selected from vesicular stomatitis glycoprotein (VSV-G), measles virus glycoprotein H, measles virus glycoprotein F, rabies virus glycoprotein (RVG), gibbon ape leukemia virus glycoprotein (GaLV), amphotropic murine leukemia virus glycoprotein (MLV-A), feline endogenous virus (RD 114) glycoprotein, fowl plague virus (FPV) glycoprotein, Ebola virus (EboV) glycoprotein, vesicular stomatitis virus (VSV) glycoprotein, and lymphocytic choriomeningitis virus (LCMV) glycoprotein.

18. The population of any one of claims 1 to 17, wherein the particles further comprise a cargo entity.

19. The population of claim 18, wherein the cargo entity is a polypeptide cargo entity.

20. The population of claim 18, wherein the cargo entity is a nucleic acid cargo entity.

21. The population of claim 18, wherein the cargo entity is part of the targeting chimeric polypeptide so that the targeting chimeric polypeptide comprises the targeting domain, the transmembrane domain, and the polypeptide cargo entity.

22. The population of claim 19, wherein the cargo entity is part of the fusogen entity polypeptide, so that the fusogen entity polypeptide comprises a fusogen moiety and the polypeptide cargo entity.

23. The population of claim 19, wherein the targeting chimeric polypeptide further includes a first polypeptide cargo entity, and the fusogen entity polypeptide comprises a fusogen moiety and a second polypeptide cargo entity.

24. The population of claim 18, wherein the polypeptide cargo entity is or is part of a distinct polypeptide from each of the targeting chimeric polypeptide and the fusogen entity polypeptide.

25. The population of claim 24, wherein the polypeptide carto entity is linked to a cargo-loading domain.

26. The population of claim 25, wherein the cargo-loading domain comprises an abscisic acid-insensitive 1 (ABI1) sequence.

27. The population of claim 24, wherein the polypeptide cargo entity further comprises a transmembrane domain.

28. The population of claim 24, wherein the polypeptide cargo entity binds to a membrane-internal portion of the targeting chimeric polypeptide, or of the fusogen entity polypeptide.

29. The population of claim 20, wherein the nucleic acid cargo entity binds to a membrane-associated polypeptide associated with the particle membrane.

30. The population of claim 29, wherein the membrane-associated polypeptide is or comprises the targeting chimeric polypeptide or the fusogen entity polypeptide.

31. The population of claims 1 to 30, wherein one or both of the targeting chimeric polypeptide and the fusogen entity polypeptide further comprise a membrane-internal cargo binding moiety.

32. The population of claim 31, wherein the cargo binding moiety binds to a polypeptide cargo.

33. The population of claim 31, wherein the cargo binding moiety binds to a nucleic acid cargo.

34. The population of claims 1 to 33, wherein the particle further comprises chimeric loading polypeptide that comprises cargo-loading domain comprising an abscisic acid-insensitive 1 (ABI1) sequence.

35. The population of claim 34, wherein the chimeric loading polypeptide further comprises a linker that connects the cargo entity and the cargo-loading domain.

36. The population of claim 35, wherein the linker of the chimeric loading polypeptide comprises:
(1) an amino acid sequence selected from SEQ ID NO: 10 (TSGGGGSGGGSGGGS), SEQ ID NO: 12 (TRGGGGSGGGSGGGS), SEQ ID NO: 14 (GGGGSGGGSGGGSTG), SEQ ID NO: 15 (DQSNSEEAKKEEAKKEEAKKSNS), SEQ ID NO: 16 (SGGGSGGGSGGGSGGSGGSGGGSGGSGGSGGGSGGGSGGG), and SEQ ID NO: 17 (ESKYGPPAPPAP); or
(2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 10, 12, 14, 15, 16, or 17.

37. The population of any of claims 34 to 36, wherein the cargo-loading domain of the chimeric loading polypeptide is a truncated variant of a wild-type protein that comprises an extracellular vesicle targeting domain.

38. The population of any one of claims 34 to 37, wherein the cargo-loading domain of the chimeric loading polypeptide comprises residues 126-423 of wild type ABI1.

39. The population of any one of claims 34 to 38, wherein the cargo-loading domain of the chimeric loading polypeptide comprises:
(SEQ ID NO: 6)
MTRVPLYGFTSICGRRPEMEAAVSTIPRFLQSSSGSMLDGRFDPQSAAHF
FGVYDGHGGSQVANYCRERMHLALAEEIAKEKPMLCDGDTWLEKWKKALF
NSFLRVDSEIESVAPETVGSTSVVAVVFPSHIFVANCGDSRAVLCRGKTA
LPLSVDHKPDREDEAARIEAAGGKVIQWNGARVFGVLAMSRSIGDRYLKP
SIIPDPEVTAVKRVKEDDCLILASDGVWDVMTDEEACEMARKRILLWHKK
NAVAGDASLLADERRKEGKDPAAMSAAEYLSKLAIQRGSKDNISVVVVDL
K,
(SEQ ID NO: 7)
VPLYGFTSICGRRPEMEAAVSTIPRFLQSSSGSMLDGRFDPQSAAHFFGV
YDGHGGSQVANYCRERMHLALAEEIAKEKPMLCDGDTWLEKWKKALFNSF
LRVDSEIESVAPETVGSTSVVAVVFPSHIFVANCGDSRAVLCRGKTALPL
SVDHKPDREDEAARIEAAGGKVIQWNGARVFGVLAMSRSIGDRYLKPSII
PDPEVTAVKRVKEDDCLILASDGVWDVMTDEEACEMARKRILLWHKKNAV
AGDASLLADERRKEGKDPAAMSAAEYLSKLAIQRGSKDNISVVVVDLK,
a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 6 or 7, or
a functional fragment of SEQ ID NO: 6, SEQ ID NO: 7, or a variant amino acid sequence thereof.

40. The population of any one of claims 34 to 39, wherein the cargo entity of the chimeric loading polypeptide is a cytosolic cargo molecule.

41. The population of any one of claims 39 to 40, wherein the first cargo entity is an ABA-binding sequence comprising a pyrabactin resistance 1-like (PYL1) sequence.

42. The population of claim 41, wherein the PYL1 sequence comprises residues 33-209 of wild type PYL1.

43. The population of claim 41 or 42, wherein the PYL1 sequence comprises
(SEQ ID NO: 2)
MGGGAPTQDEFTQLSQSIAEFHTYQLGNGRCSSLLAQRIHAPPETVWSVV
RRFDRPQIYKHFIKSCNVSEDFEMRVGCTRDVNVISGLPANTSRERLDLL
DDDRRVTGFSITGGEHRLRNYKSVTTVHRFEKEEEEERIWTVVLESYVVD
VPEGNSEEDTRLFADTVIRLNLQKLASITEAMN,
(SEQ ID NO: 3)
TQDEFTQLSQSIAEFHTYQLGNGRCSSLLAQRIHAPPETVWSVVRRFDRP
QIYKHFIKSCNVSEDFEMRVGCTRDVNVISGLPANTSRERLDLLDDDRRV
TGFSITGGEHRLRNYKSVTTVHRFEKEEEEERIWTVVLESYVVDVPEGNS
EEDTRLFADTVIRLNLQKLASITEAMN,
or
a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 2 or 3, or
a functional fragment of SEQ ID NO: 2, SEQ ID NO: 3, or a variant amino acid sequence thereof.

44. The population of any one of claims 39-43, wherein the CDMP further comprises abscisic acid (ABA).

45. The population of any one of claims 11 to 44, wherein the CDMP encompasses or contains within it a viral nucleocapsid, a synthetic nucleic acid, a transcription factor, a recombinase, a base editor, prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex.

46. The population of claims 1-45, wherein the population is frozen.

47. The population of claim 46, wherein the population is frozen in glycerol, or dried, or in culture.

48. A targeting chimeric polypeptide comprising:
(a) a targeting domain comprising a binding moiety that specifically binds to a target ligand on surfaces of recipient cells of interest; linked directly or indirectly with
(b) a transmembrane domain.

49. The targeting chimeric polypeptide of claim 46, wherein the targeting domain is or comprises an antibody agent.

50. The targeting chimeric polypeptide of claim 47, wherein the antibody agent is a single chain antibody agent.

51. The targeting chimeric polypeptide of claim 47, wherein the antibody agent is selected from the group consisting of an antibody, a Fab, a Fab′, a F(ab′)₂, a Fd, a scFv, a single-chain antibody, a disulfide-linked Fvs (sdFv), an affinibody, a DARPIN, a nanobody, a variable lymphocyte receptor (VLR), and a camelid antibody.

52. The targeting chimeric polypeptide of claim 46, wherein the targeting domain is or comprises a DARPIN or other engineered high affinity binding polypeptides.

53. The targeting chimeric polypeptide of claim 49, wherein the targeting domain is a scFv.

54. The targeting chimeric polypeptide of claims 46 to 51, wherein the transmembrane domain comprises AVGQDTQEVIVVPHSLPFKVVVISAILALVVLTIISLIILIMLWQKKPR (SEQ ID NO: 18), a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to SEQ ID NO: 18, or a functional fragment thereof.

55. The targeting chimeric polypeptide of any one of claims 46-52, wherein the targeting domain comprises an amino acid sequence of
(SEQ ID NO: 20)
NIMMTQSPSSLAVSAGEKVTMTCKSSQSVLYSSNQKNYLAWYQQKPGQSP
KLLIYWASTRESGVPDRFTGSGSGTDFTLTISSVQPEDLAVYYCHQYLSS
HTFGGGTKLEIKRGGGGSGGGGSGGGGSQLQQPGAELVRPGSSVKLSCKA
SGYTFTRYWIHWVKQRPIQGLEWIGNIDPSDSETHYNQKFKDKATLTVDK
SSGTAYMQLSSLTSEDSAVYYCATEDLYYAMEYWGQGTSVTVSS.

56. The targeting chimeric polypeptide of any one of claims 46-53 further comprising a first cargo entity connected to the transmembrane domain via a linker.

57. The targeting chimeric polypeptide of claim 46-54, wherein the linker comprises:
(1) an amino acid sequence selected from SEQ ID NO: 10 (TSGGGGSGGGSGGGS), SEQ ID NO: 12 (TRGGGGSGGGSGGGS), SEQ ID NO: 14 (GGGGSGGGSGGGSTG), SEQ ID NO: 15 (DQSNSEEAKKEEAKKEEAKKSNS), SEQ ID NO: 16 (SGGGSGGGSGGGSGGSGGSGGGSGGSGGSGGGSGGGSGGG), and SEQ ID NO: 17 (ESKYGPPAPPAP); or
(2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% sequence identity to any one of SEQ ID NOs: 10, 12, 14, 15, 16, or 17.

58. A system comprising
a first polynucleotide encoding a fusogen entity polypeptide; and
a second polynucleotide encoding a targeting chimeric polypeptide.

59. The system of claim 58, wherein the first and second polynucleotides are part of a single polynucleotide molecule.

60. The system of claims 58-59 wherein the first and second polynucleotides are separate polynucleotide molecules.

61. The system of claims 58-60, wherein the at least one of the first and second polynucleotides are circular.

62. The system of claims 58-61, wherein both of the first and second polynucleotides are circular.

63. The system of claims 58-62, wherein the at least one of the first and second polynucleotides are linear.

64. The system of claims 58-63, wherein both of the first and second polynucleotides are linear.

65. The system of claims 58-64, wherein the first and second polynucleotides are disposed in separate containers.

66. The system of claims 58-64 wherein the first and second polynucleotides are disposed in the same container.

67. The system of claims 58-66, wherein the first and second polynucleotides include expression elements sufficient to direct production of the encoded polypeptide in an engineered production cell.

68. The system of claim 57, wherein the cell is a mammalian cell.

69. The system of claims 67-68, wherein the expression elements are promoters.

70. The system of claim 69, wherein the promoter is a small molecule-inducible promoter.

71. The system of claims 58-70, wherein the fusogen entity polypeptide comprises:
a fusogen moiety; and
a transmembrane domain.

72. The system of claim 71, wherein the fusogen moiety and the membrane association portion are directly linked or indirectly linked via a linker.

73. The system of claims 71-72, wherein the fusogen moiety is characterized by an ability to promote fusion between lipid bilayers.

74. The system of claims 71-73, wherein the fusogen moiety is a viral fusogen moiety.

75. The system of claims 71-73, wherein the fusogen moiety is a non-viral fusogen moiety.

76. The system of claims 71-75, wherein the transmembrane domain is a viral transmembrane domain or derivative thereof.

77. The system of claims 71-75, wherein the transmembrane domain is a non-viral transmembrane domain.

78. The system of claims 58-77, wherein the targeting chimeric polypeptide comprises:
(a) a targeting domain comprising a binding moiety that specifically binds to a target ligand on surfaces of recipient cells of interest; and
(b) a transmembrane domain.

79. The system of claim 78, wherein the affinity moiety and the membrane association portion are directly linked or indirectly linked via a linker.

80. A nucleic acid encoding the chimeric targeting polypeptide and/or fusion entity polypeptide according to any one of claims 1-45.

81. A cell comprising the population of any one of claims 1-45 or the nucleic acid of claim 80.

82. The cell of claim 81, wherein the cell is a mammalian cell, wherein the mammalian cell is optionally selected from HEK293, HEK293FT, a mesenchymal stem cell, a megakaryocyte, an induced pluripotent stem cell (iPSC), a T cell, an erythrocyte, an erythropoetic precursor, and an iPSC-derived version of any of the preceding cells.

83. A method of producing a lymphocyte-targeting lipid bilayer particle, comprising culturing the cell of claim 23 or 24, and harvesting lipid bilayer particle produced by the cell.

84. A method of targeting delivery of a cargo entity to a lymphocyte, comprising administering to an individual the population according to any one of claims 7-21, wherein the lipid bilayer particle comprises a cargo molecule.

85. The method of claim 84, wherein the cargo entity comprises a viral nucleocapsid, a synthetic nucleic acid, a transcription factor, a recombinase, a base editor, a prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex.

86. The method of claim 84 or 85, wherein the cargo entity comprises a nucleic acid sequence encoding a chimeric antigen receptor.

87. An ex vivo method of targeting delivery of a cargo entity to a lymphocyte, comprising obtaining a population of lymophcytes from an individual, and contacting the population of lymphocytes ex vivo with the population according to any one of claims 7-21, wherein the lipid bilayer particle comprises a cargo molecule.

88. The method of claim 87, wherein the cargo entity comprises a viral nucleocapsid, a synthetic nucleic acid, a transcription factor, a recombinase, a base editor, a prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex.

89. The method of claim 87 or 88, wherein the cargo entity comprises a nucleic acid sequence encoding a chimeric antigen receptor.

90. The method of any one of claims 87-89, wherein the population of lymphocytes were obtained via apheresis.

91. The method of any one of claims 87-90 further comprising administering the population of lymphocytes back into the individual after the lymphocytes have been contacted with the lipid bilayer particle.

92. A method of manufacturing a population of lipid bilayer particles, the method comprising a step of:
isolating lipid bilayer particles produced by engineered production cells engineered to express
a fusogen entity polypeptide; and
targeting entity polypeptide,
such that the lipid bilayer particles contain the fusogen entity and targeting chimeric polypeptides on their surfaces.

93. A method of delivering a cargo entity to a recipient cell, the method comprising a step of:
contacting the recipient cell with a population of lipid bilayer particles prepared from a engineered production cell, which engineered production cell was engineered to express
a fusogen entity polypeptide; and
a targeting chimeric polypeptide,
such that the lipid bilayer particles contain the fusogen entity and targeting chimeric polypeptides on their surfaces.

94. A method of manufacturing a production cell, the method comprising a step pf:
engineering the production cell to express
a fusogen entity polypeptide; and
a targeting chimeric polypeptide,
such that the production cell produced lipid bilayer particles that contain the fusogen entity and targeting chimeric polypeptides on their surfaces.

95. A recipient cell containing a cargo entity and wherein the recipient cell membrane includes a targeting chimeric polypeptide and a fusogen entity polypeptide, and a nucleus.
